SEQUENCE LISTING (57 sequences)

SEQ ID NO: 1 - RNA, 3637 nt, Homo sapiens
  source 1..3637: mol_type = mRNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 2 - RNA, 22 nt, synthetic construct
  source 1..22: mol_type = other RNA
ttatcttcaagttacaaaagca

SEQ ID NO: 3 - RNA, 22 nt, synthetic construct
  source 1..22: mol_type = other RNA
tgaataaatatcttcaagttac

SEQ ID NO: 4 - RNA, 22 nt, synthetic construct
  source 1..22: mol_type = other RNA
taaaaatgctacaaaacccaga

SEQ ID NO: 5 - RNA, 22 nt, synthetic construct
  source 1..22: mol_type = other RNA
taataaaaatgctacaaaaccc

SEQ ID NO: 6 - RNA, 22 nt, synthetic construct
  source 1..22: mol_type = other RNA
tttaataaaaatgctacaaaac

SEQ ID NO: 7 - RNA, 22 nt, synthetic construct
  source 1..22: mol_type = other RNA
ttattaataaaaatgctacaaa

SEQ ID NO: 8 - RNA, 20 nt, synthetic construct
  source 1..20: mol_type = other RNA
cttttgtaacttgaagataa

SEQ ID NO: 9 - RNA, 20 nt, synthetic construct
  source 1..20: mol_type = other RNA
aacttgaagatatttattca

SEQ ID NO: 10 - RNA, 20 nt, synthetic construct
  source 1..20: mol_type = other RNA
tgggttttgtagcattttta

SEQ ID NO: 11 - RNA, 20 nt, synthetic construct
  source 1..20: mol_type = other RNA
gttttgtagcatttttatta

SEQ ID NO: 12 - RNA, 20 nt, synthetic construct
  source 1..20: mol_type = other RNA
tttgtagcatttttattaaa

SEQ ID NO: 13 - RNA, 20 nt, synthetic construct
  source 1..20: mol_type = other RNA
tgtagcatttttattaataa

SEQ ID NO: 14-15 - skipped (2 entries)

SEQ ID NO: 16 - RNA, 20 nt, synthetic construct
  modified_base 1..3: mod_base = OTHER; note = linked via phosphorothioate
  modified_base 1: mod_base = cm
  modified_base 2: mod_base = um
  modified_base 3: mod_base = um
  modified_base 4: mod_base = um
  modified_base 5: mod_base = um
  modified_base 6: mod_base = OTHER; note = 2'-fluoro-guanosine
  modified_base 7: mod_base = um
  modified_base 8: mod_base = OTHER; note = 2'-fluoro-adenosine
  modified_base 9: mod_base = OTHER; note = 2'-fluoro-adenosine
  modified_base 10: mod_base = OTHER; note = 2'-fluoro-cytidine
  modified_base 11: mod_base = OTHER; note = 2'-fluoro-uridine
  modified_base 12: mod_base = um
  modified_base 13: mod_base = gm
  modified_base 14: mod_base = OTHER; note = 2'-O-methyl-adenosine
  modified_base 15: mod_base = OTHER; note = 2'-O-methyl-adenosine
  modified_base 16: mod_base = gm
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
cttttgtaacttgaagataa

SEQ ID NO: 17 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-uridine
  modified_base 3: mod_base = OTHER; note = 2'-fluoro-adenosine
  modified_base 4: mod_base = um
  modified_base 5: mod_base = OTHER; note = 2'-fluoro-cytidine
  modified_base 6: mod_base = um
  modified_base 7: mod_base = OTHER; note = 2'-fluoro-uridine
  modified_base 8: mod_base = cm
  modified_base 9: mod_base = OTHER; note = 2'-O-methyl-adenosine
  modified_base 10: mod_base = OTHER; note = 2'-fluoro-adenosine
  modified_base 11: mod_base = gm
  modified_base 12: mod_base = um
  modified_base 13: mod_base = um
  modified_base 14: mod_base = OTHER; note = 2'-fluoro-adenosine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = gm
  modified_base 21: mod_base = cm
  modified_base 22: mod_base = OTHER; note = 2'-O-methyl-adenosine
  source 1..22: mol_type = other RNA
ttatcttcaagttacaaaagca

SEQ ID NO: 18 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = OTHER; note = 2'-O-methyl-adenosine
  modified_base 3: mod_base = cm
  modified_base 4: mod_base = um
  modified_base 5: mod_base = um
  modified_base 6: mod_base = OTHER; note = 2'-fluoro-guanosine
  modified_base 7: mod_base = OTHER; note = 2'-O-methyl-adenosine
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-adenosine
  modified_base 11: mod_base = OTHER; note = 2'-fluoro-uridine
  modified_base 12: mod_base = OTHER; note = 2'-O-methyl-adenosine
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18..20: mod_base = OTHER; note = linked via phosphorothioate
  modified_base 18: mod_base = um
  modified_base 19: mod_base = cm
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
aacttgaagatatttattca

SEQ ID NO: 19 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-guanosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = um
  modified_base 10: mod_base = OTHER; note = 2'-fluoro-adenosine
  modified_base 11: mod_base = um
  modified_base 12: mod_base = cm
  modified_base 13: mod_base = um
  modified_base 14: mod_base = OTHER; note = 2'-fluoro-uridine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = gm
  modified_base 19: mod_base = um
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = cm
  source 1..22: mol_type = other RNA
tgaataaatatcttcaagttac

SEQ ID NO: 20 - RNA, 20 nt, synthetic construct
  modified_base 1..3: mod_base = OTHER; note = linked via phosphorothioate
  modified_base 1: mod_base = um
  modified_base 2: mod_base = gm
  modified_base 3: mod_base = gm
  modified_base 4: mod_base = gm
  modified_base 5: mod_base = um
  modified_base 6: mod_base = OTHER; note = 2'-fluoro-uridine
  modified_base 7: mod_base = um
  modified_base 8: mod_base = OTHER; note = 2'-fluoro-uridine
  modified_base 9: mod_base = OTHER; note = 2'-fluoro-guanosine
  modified_base 10: mod_base = OTHER; note = 2'-fluoro-uridine
  modified_base 11: mod_base = OTHER; note = 2'-fluoro-adenosine
  modified_base 12: mod_base = gm
  modified_base 13: mod_base = cm
  modified_base 14: mod_base = OTHER; note = 2'-O-methyl-adenosine
  modified_base 15: mod_base = um
  modified_base 16: mod_base = um
  modified_base 17: mod_base = um
  modified_base 18..20: mod_base = OTHER; note = linked via phosphorothioate
  modified_base 18: mod_base = um
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
tgggttttgtagcattttta

SEQ ID NO: 21 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-adenosine
  modified_base 6: mod_base = OTHER; note = 2'-O-methyl-adenosine
  modified_base 7: mod_base = OTHER; note = 2'-fluoro-uridine
  modified_base 8: mod_base = gm
  modified_base 9: mod_base = cm
  modified_base 10: mod_base = OTHER; note = 2'-fluoro-uridine
  modified_base 11: mod_base = OTHER; note = 2'-O-methyl-adenosine
  modified_base 12: mod_base = cm
  modified_base 13: mod_base = OTHER; note = 2'-O-methyl-adenosine
  modified_base 14: mod_base = OTHER; note = 2'-fluoro-adenosine
  modified_base 15: mod_base = OTHER; note = 2'-O-methyl-adenosine
  modified_base 16: mod_base = OTHER; note = 2'-fluoro-adenosine
  modified_base 17: mod_base = cm
  modified_base 18: mod_base = cm
  modified_base 19: mod_base = cm
  modified_base 20..22: mod_base = OTHER; note = linked via phosphorothioate
  modified_base 20: mod_base = OTHER; note = 2'-O-methyl-adenosine
  modified_base 21: mod_base = gm
  modified_base 22: mod_base = OTHER; note = 2'-O-methyl-adenosine
  source 1..22: mol_type = other RNA
taaaaatgctacaaaacccaga

SEQ ID NO: 22 - RNA, 20 nt, synthetic construct
  modified_base 1..3: mod_base = OTHER; note = linked via phosphorothioate
  modified_base 1: mod_base = gm
  modified_base 2: mod_base = um
  modified_base 3: mod_base = um
  modified_base 4: mod_base = um
  modified_base 5: mod_base = um
  modified_base 6: mod_base = OTHER; note = 2'-fluoro-guanosine
  modified_base 7: mod_base = um
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-cytidine
  modified_base 11: mod_base = OTHER; note = 2'-fluoro-adenosine
  modified_base 12: mod_base = um
  modified_base 13: mod_base = um
  modified_base 14: mod_base = um
  modified_base 15: mod_base = um
  modified_base 16: mod_base = um
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
gttttgtagcatttttatta

SEQ ID NO: 23 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-adenosine
  modified_base 3: mod_base = OTHER; note = 2'-fluoro-adenosine
  modified_base 4: mod_base = um
  modified_base 5: mod_base = OTHER; note = 2'-fluoro-adenosine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-uridine
  modified_base 11: mod_base = gm
  modified_base 12: mod_base = cm
  modified_base 13: mod_base = um
  modified_base 14: mod_base = OTHER; note = 2'-fluoro-adenosine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = cm
  modified_base 21: mod_base = cm
  modified_base 22: mod_base = cm
  source 1..22: mol_type = other RNA
taataaaaatgctacaaaaccc

SEQ ID NO: 24 - RNA, 20 nt, synthetic construct
  modified_base 1..3: mod_base = OTHER; note = linked via phosphorothioate
  modified_base 1: mod_base = um
  modified_base 2: mod_base = um
  modified_base 3: mod_base = um
  modified_base 4: mod_base = gm
  modified_base 5: mod_base = um
  modified_base 6: mod_base = OTHER; note = 2'-fluoro-adenosine
  modified_base 7: mod_base = gm
  modified_base 8: mod_base = OTHER; note = 2'-fluoro-cytidine
  modified_base 9: mod_base = OTHER; note = 2'-fluoro-adenosine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = um
  modified_base 14: mod_base = um
  modified_base 15: mod_base = OTHER; note = 2'-O-methyl-adenosine
  modified_base 16: mod_base = um
  modified_base 17: mod_base = um
  modified_base 18..20: mod_base = OTHER; note = linked via phosphorothioate
  modified_base 18: mod_base = OTHER; note = 2'-O-methyl-adenosine
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
tttgtagcatttttattaaa

SEQ ID NO: 25 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-uridine
  modified_base 3: mod_base = OTHER; note = 2'-fluoro-uridine
  modified_base 4: mod_base = OTHER; note = 2'-O-methyl-adenosine
  modified_base 5: mod_base = OTHER; note = 2'-fluoro-adenosine
  modified_base 6: mod_base = um
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = um
  modified_base 13: mod_base = gm
  modified_base 14: mod_base = OTHER; note = 2'-fluoro-cytidine
  modified_base 15: mod_base = um
  modified_base 16: mod_base = OTHER; note = 2'-fluoro-adenosine
  modified_base 17: mod_base = cm
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = OTHER; note = 2'-O-methyl-adenosine
  modified_base 21: mod_base = OTHER; note = 2'-O-methyl-adenosine
  modified_base 22: mod_base = cm
  source 1..22: mol_type = other RNA
tttaataaaaatgctacaaaac

SEQ ID NO: 26 - RNA, 20 nt, synthetic construct
  modified_base 1..3: mod_base = OTHER; note = linked via phosphorothioate
  modified_base 1: mod_base = um
  modified_base 2: mod_base = gm
  modified_base 3: mod_base = um
  modified_base 4: mod_base = OTHER; note = 2'-O-methyl-adenosine
  modified_base 5: mod_base = gm
  modified_base 6: mod_base = OTHER; note = 2'-fluoro-cytidine
  modified_base 7: mod_base = OTHER; note = 2'-O-methyl-adenosine
  modified_base 8: mod_base = OTHER; note = 2'-fluoro-uridine
  modified_base 9: mod_base = OTHER; note = 2'-fluoro-uridine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = OTHER; note = 2'-O-methyl-adenosine
  modified_base 14: mod_base = um
  modified_base 15: mod_base = um
  modified_base 16: mod_base = OTHER; note = 2'-O-methyl-adenosine
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
tgtagcatttttattaataa

SEQ ID NO: 27 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-uridine
  modified_base 3: mod_base = OTHER; note = 2'-fluoro-adenosine
  modified_base 4: mod_base = um
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = um
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = OTHER; note = 2'-O-methyl-adenosine
  modified_base 14: mod_base = OTHER; note = 2'-fluoro-uridine
  modified_base 15: mod_base = gm
  modified_base 16: mod_base = OTHER; note = 2'-fluoro-cytidine
  modified_base 17: mod_base = um
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = OTHER; note = 2'-O-methyl-adenosine
  modified_base 21: mod_base = OTHER; note = 2'-O-methyl-adenosine
  modified_base 22: mod_base = OTHER; note = 2'-O-methyl-adenosine
  source 1..22: mol_type = other RNA
ttattaataaaaatgctacaaa

SEQ ID NO: 28 - RNA, 22 nt, synthetic construct
  source 1..22: mol_type = other RNA
tatgctacaaaacccagaataa

SEQ ID NO: 29 - RNA, 22 nt, synthetic construct
  source 1..22: mol_type = other RNA
ttaataaaaatgctacaaaacc

SEQ ID NO: 30 - RNA, 20 nt, synthetic construct
  source 1..20: mol_type = other RNA
attctgggttttgtagcata

SEQ ID NO: 31 - RNA, 20 nt, synthetic construct
  source 1..20: mol_type = other RNA
ttttgtagcatttttattaa

SEQ ID NO: 32 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = um
  modified_base 3: mod_base = um
  modified_base 4: mod_base = cm
  modified_base 5: mod_base = um
  modified_base 6: mod_base = OTHER; note = 2'-fluoro-guanosine
  modified_base 7: mod_base = gm
  modified_base 8: mod_base = OTHER; note = 2'-fluoro-guanosine
  modified_base 9: mod_base = OTHER; note = 2'-fluoro-uridine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = gm
  modified_base 14: mod_base = um
  modified_base 15: mod_base = OTHER; note = 2'-O-methyl-adenosine
  modified_base 16: mod_base = gm
  modified_base 17: mod_base = cm
  modified_base 18..20: mod_base = OTHER; note = linked via phosphorothioate
  modified_base 18: mod_base = OTHER; note = 2'-O-methyl-adenosine
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
attctgggttttgtagcata

SEQ ID NO: 33 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-adenosine
  modified_base 3: mod_base = OTHER; note = 2'-fluoro-uridine
  modified_base 4: mod_base = gm
  modified_base 5: mod_base = OTHER; note = 2'-fluoro-cytidine
  modified_base 6: mod_base = um
  modified_base 7: mod_base = OTHER; note = 2'-fluoro-adenosine
  modified_base 8: mod_base = cm
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = cm
  modified_base 14: mod_base = OTHER; note = 2'-fluoro-cytidine
  modified_base 15: mod_base = cm
  modified_base 16: mod_base = OTHER; note = 2'-fluoro-adenosine
  modified_base 17: mod_base = gm
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = OTHER; note = 2'-O-methyl-adenosine
  source 1..22: mol_type = other RNA
tatgctacaaaacccagaataa

SEQ ID NO: 34 - RNA, 20 nt, synthetic construct
  modified_base 1..3: mod_base = OTHER; note = linked via phosphorothioate
  modified_base 1: mod_base = um
  modified_base 2: mod_base = um
  modified_base 3: mod_base = um
  modified_base 4: mod_base = um
  modified_base 5: mod_base = gm
  modified_base 6: mod_base = OTHER; note = 2'-fluoro-uridine
  modified_base 7: mod_base = OTHER; note = 2'-O-methyl-adenosine
  modified_base 8: mod_base = OTHER; note = 2'-fluoro-guanosine
  modified_base 9: mod_base = OTHER; note = 2'-fluoro-cytidine
  modified_base 10: mod_base = OTHER; note = 2'-fluoro-adenosine
  modified_base 11: mod_base = OTHER; note = 2'-fluoro-uridine
  modified_base 12: mod_base = um
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
ttttgtagcatttttattaa

SEQ ID NO: 35 - RNA, 22 nt, synthetic construct
  modified_base 1..3: mod_base = OTHER; note = linked via phosphorothioate
  modified_base 1: mod_base = OTHER; note = mono methyl protected phosphate mimic linked uridine
  modified_base 2: mod_base = OTHER; note = 2'-fluoro-uridine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-adenosine
  modified_base 11: mod_base = um
  modified_base 12: mod_base = gm
  modified_base 13: mod_base = cm
  modified_base 14: mod_base = OTHER; note = 2'-fluoro-uridine
  modified_base 15: mod_base = OTHER; note = 2'-O-methyl-adenosine
  modified_base 16: mod_base = OTHER; note = 2'-fluoro-cytidine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = OTHER; note = 2'-O-methyl-adenosine
  modified_base 21: mod_base = cm
  modified_base 22: mod_base = cm
  source 1..22: mol_type = other RNA
ttaataaaaatgctacaaaacc

SEQ ID NO: 36 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-uridine
  modified_base 3: mod_base = OTHER; note = 2'-fluoro-adenosine
  modified_base 4: mod_base = um
  modified_base 5: mod_base = OTHER; note = 2'-fluoro-cytidine
  modified_base 6: mod_base = um
  modified_base 7: mod_base = OTHER; note = 2'-fluoro-uridine
  modified_base 8: mod_base = cm
  modified_base 9: mod_base = OTHER; note = 2'-O-methyl-adenosine
  modified_base 10: mod_base = OTHER; note = 2'-fluoro-adenosine
  modified_base 11: mod_base = gm
  modified_base 12: mod_base = um
  modified_base 13: mod_base = um
  modified_base 14: mod_base = OTHER; note = 2'-fluoro-adenosine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = gm
  modified_base 21: mod_base = cm
  modified_base 22: mod_base = OTHER; note = 2'-O-methyl-adenosine
  source 1..22: mol_type = other RNA
ttatcttcaagttacaaaagca

SEQ ID NO: 37 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-guanosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = um
  modified_base 10: mod_base = OTHER; note = 2'-fluoro-adenosine
  modified_base 11: mod_base = um
  modified_base 12: mod_base = cm
  modified_base 13: mod_base = um
  modified_base 14: mod_base = OTHER; note = 2'-fluoro-uridine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = gm
  modified_base 19: mod_base = um
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = cm
  source 1..22: mol_type = other RNA
tgaataaatatcttcaagttac

SEQ ID NO: 38 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-adenosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-adenosine
  modified_base 6: mod_base = OTHER; note = 2'-O-methyl-adenosine
  modified_base 7: mod_base = OTHER; note = 2'-fluoro-uridine
  modified_base 8: mod_base = gm
  modified_base 9: mod_base = cm
  modified_base 10: mod_base = OTHER; note = 2'-fluoro-uridine
  modified_base 11: mod_base = OTHER; note = 2'-O-methyl-adenosine
  modified_base 12: mod_base = cm
  modified_base 13: mod_base = OTHER; note = 2'-O-methyl-adenosine
  modified_base 14: mod_base = OTHER; note = 2'-fluoro-adenosine
  modified_base 15: mod_base = OTHER; note = 2'-O-methyl-adenosine
  modified_base 16: mod_base = OTHER; note = 2'-fluoro-adenosine
  modified_base 17: mod_base = cm
  modified_base 18: mod_base = cm
  modified_base 19: mod_base = cm
  modified_base 20..22: mod_base = OTHER; note = linked via phosphorothioate
  modified_base 20: mod_base = OTHER; note = 2'-O-methyl-adenosine
  modified_base 21: mod_base = gm
  modified_base 22: mod_base = OTHER; note = 2'-O-methyl-adenosine
  source 1..22: mol_type = other RNA
taaaaatgctacaaaacccaga

SEQ ID NO: 39 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-adenosine
  modified_base 3: mod_base = OTHER; note = 2'-fluoro-adenosine
  modified_base 4: mod_base = um
  modified_base 5: mod_base = OTHER; note = 2'-fluoro-adenosine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-uridine
  modified_base 11: mod_base = gm
  modified_base 12: mod_base = cm
  modified_base 13: mod_base = um
  modified_base 14: mod_base = OTHER; note = 2'-fluoro-adenosine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = cm
  modified_base 21: mod_base = cm
  modified_base 22: mod_base = cm
  source 1..22: mol_type = other RNA
taataaaaatgctacaaaaccc

SEQ ID NO: 40 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-uridine
  modified_base 3: mod_base = OTHER; note = 2'-fluoro-uridine
  modified_base 4: mod_base = OTHER; note = 2'-O-methyl-adenosine
  modified_base 5: mod_base = OTHER; note = 2'-fluoro-adenosine
  modified_base 6: mod_base = um
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = um
  modified_base 13: mod_base = gm
  modified_base 14: mod_base = OTHER; note = 2'-fluoro-cytidine
  modified_base 15: mod_base = um
  modified_base 16: mod_base = OTHER; note = 2'-fluoro-adenosine
  modified_base 17: mod_base = cm
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = OTHER; note = 2'-O-methyl-adenosine
  modified_base 21: mod_base = OTHER; note = 2'-O-methyl-adenosine
  modified_base 22: mod_base = cm
  source 1..22: mol_type = other RNA
tttaataaaaatgctacaaaac

SEQ ID NO: 41 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-uridine
  modified_base 3: mod_base = OTHER; note = 2'-fluoro-adenosine
  modified_base 4: mod_base = um
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = um
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = OTHER; note = 2'-O-methyl-adenosine
  modified_base 14: mod_base = OTHER; note = 2'-fluoro-uridine
  modified_base 15: mod_base = gm
  modified_base 16: mod_base = OTHER; note = 2'-fluoro-cytidine
  modified_base 17: mod_base = um
  modified_base 18: mod_base = OTHER; note = 2'-O-methyl-adenosine
  modified_base 19: mod_base = cm
  modified_base 20..22: mod_base = OTHER; note = linked via phosphorothioate
  modified_base 20: mod_base = OTHER; note = 2'-O-methyl-adenosine
  modified_base 21: mod_base = OTHER; note = 2'-O-methyl-adenosine
  modified_base 22: mod_base = OTHER; note = 2'-O-methyl-adenosine
  source 1..22: mol_type = other RNA
ttattaataaaaatgctacaaa

SEQ ID NO: 42 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-adenosine
  modified_base 3: mod_base = OTHER; note = 2'-fluoro-uridine
  modified_base 4: mod_base = gm
  modified_base 5: mod_base = OTHER; note = 2'-fluoro-cytidine
  modified_base 6: mod_base = um
  modified_base 7: mod_base = OTHER; note = 2'-fluoro-adenosine
  modified_base 8: mod_base = cm
  modified_base 9: mod_base = OTHER; note = 2'-O-methyl-adenosine
  modified_base 10: mod_base = OTHER; note = 2'-fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = cm
  modified_base 14: mod_base = OTHER; note = 2'-fluoro-cytidine
  modified_base 15: mod_base = cm
  modified_base 16: mod_base = OTHER; note = 2'-fluoro-adenosine
  modified_base 17: mod_base = gm
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = OTHER; note = 2'-O-methyl-adenosine
  source 1..22: mol_type = other RNA
tatgctacaaaacccagaataa

SEQ ID NO: 43 - RNA, 22 nt, synthetic construct
  modified_base 1..3: mod_base = OTHER; note = linked via phosphorothioate
  modified_base 1: mod_base = um
  modified_base 2: mod_base = OTHER; note = 2'-fluoro-uridine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = OTHER; note = 2'-O-methyl-adenosine
  modified_base 10: mod_base = OTHER; note = 2'-fluoro-adenosine
  modified_base 11: mod_base = um
  modified_base 12: mod_base = gm
  modified_base 13: mod_base = cm
  modified_base 14: mod_base = OTHER; note = 2'-fluoro-uridine
  modified_base 15: mod_base = OTHER; note = 2'-O-methyl-adenosine
  modified_base 16: mod_base = OTHER; note = 2'-fluoro-cytidine
  modified_base 17: mod_base = OTHER; note = 2'-O-methyl-adenosine
  modified_base 18: mod_base = OTHER; note = 2'-O-methyl-adenosine
  modified_base 19: mod_base = OTHER; note = 2'-O-methyl-adenosine
  modified_base 20..22: mod_base = OTHER; note = linked via phosphorothioate
  modified_base 20: mod_base = OTHER; note = 2'-O-methyl-adenosine
  modified_base 21: mod_base = cm
  modified_base 22: mod_base = cm
  source 1..22: mol_type = other RNA
ttaataaaaatgctacaaaacc

SEQ ID NO: 44 - RNA, 20 nt, synthetic construct
  modified_base 1..3: mod_base = OTHER; note = linked via phosphorothioate
  modified_base 1: mod_base = cm
  modified_base 2: mod_base = um
  modified_base 3: mod_base = um
  modified_base 4: mod_base = um
  modified_base 5: mod_base = um
  modified_base 6: mod_base = OTHER; note = 2'-fluoro-guanosine
  modified_base 7: mod_base = um
  modified_base 8: mod_base = OTHER; note = 2'-fluoro-adenosine
  modified_base 9: mod_base = OTHER; note = 2'-fluoro-adenosine
  modified_base 10: mod_base = OTHER; note = 2'-fluoro-cytidine
  modified_base 11: mod_base = OTHER; note = 2'-fluoro-uridine
  modified_base 12: mod_base = um
  modified_base 13: mod_base = gm
  modified_base 14: mod_base = OTHER; note = 2'-O-methyl-adenosine
  modified_base 15: mod_base = OTHER; note = 2'-O-methyl-adenosine
  modified_base 16: mod_base = gm
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
cttttgtaacttgaagataa

SEQ ID NO: 45 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = OTHER; note = 2'-O-methyl-adenosine
  modified_base 3: mod_base = cm
  modified_base 4: mod_base = um
  modified_base 5: mod_base = um
  modified_base 6: mod_base = OTHER; note = 2'-fluoro-guanosine
  modified_base 7: mod_base = OTHER; note = 2'-O-methyl-adenosine
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-adenosine
  modified_base 11: mod_base = OTHER; note = 2'-fluoro-uridine
  modified_base 12: mod_base = OTHER; note = 2'-O-methyl-adenosine
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18..20: mod_base = OTHER; note = linked via phosphorothioate
  modified_base 18: mod_base = um
  modified_base 19: mod_base = cm
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
aacttgaagatatttattca

SEQ ID NO: 46 - RNA, 20 nt, synthetic construct
  modified_base 1..3: mod_base = OTHER; note = linked via phosphorothioate
  modified_base 1: mod_base = um
  modified_base 2: mod_base = gm
  modified_base 3: mod_base = gm
  modified_base 4: mod_base = gm
  modified_base 5: mod_base = um
  modified_base 6: mod_base = OTHER; note = 2'-fluoro-uridine
  modified_base 7: mod_base = um
  modified_base 8: mod_base = OTHER; note = 2'-fluoro-uridine
  modified_base 9: mod_base = OTHER; note = 2'-fluoro-guanosine
  modified_base 10: mod_base = OTHER; note = 2'-fluoro-uridine
  modified_base 11: mod_base = OTHER; note = 2'-fluoro-adenosine
  modified_base 12: mod_base = gm
  modified_base 13: mod_base = cm
  modified_base 14: mod_base = OTHER; note = 2'-O-methyl-adenosine
  modified_base 15: mod_base = um
  modified_base 16: mod_base = um
  modified_base 17: mod_base = um
  modified_base 18..20: mod_base = OTHER; note = linked via phosphorothioate
  modified_base 18: mod_base = um
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
tgggttttgtagcattttta

SEQ ID NO: 47 - RNA, 20 nt, synthetic construct
  modified_base 1..3: mod_base = OTHER; note = linked via phosphorothioate
  modified_base 1: mod_base = gm
  modified_base 2: mod_base = um
  modified_base 3: mod_base = um
  modified_base 4: mod_base = um
  modified_base 5: mod_base = um
  modified_base 6: mod_base = OTHER; note = 2'-fluoro-guanosine
  modified_base 7: mod_base = um
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-cytidine
  modified_base 11: mod_base = OTHER; note = 2'-fluoro-adenosine
  modified_base 12: mod_base = um
  modified_base 13: mod_base = um
  modified_base 14: mod_base = um
  modified_base 15: mod_base = um
  modified_base 16: mod_base = um
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
gttttgtagcatttttatta

SEQ ID NO: 48 - RNA, 20 nt, synthetic construct
  modified_base 1..3: mod_base = OTHER; note = linked via phosphorothioate
  modified_base 1: mod_base = um
  modified_base 2: mod_base = um
  modified_base 3: mod_base = um
  modified_base 4: mod_base = gm
  modified_base 5: mod_base = um
  modified_base 6: mod_base = OTHER; note = 2'-fluoro-adenosine
  modified_base 7: mod_base = gm
  modified_base 8: mod_base = OTHER; note = 2'-fluoro-cytidine
  modified_base 9: mod_base = OTHER; note = 2'-fluoro-adenosine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = um
  modified_base 14: mod_base = um
  modified_base 15: mod_base = OTHER; note = 2'-O-methyl-adenosine
  modified_base 16: mod_base = um
  modified_base 17: mod_base = um
  modified_base 18..20: mod_base = OTHER; note = linked via phosphorothioate
  modified_base 18: mod_base = OTHER; note = 2'-O-methyl-adenosine
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
tttgtagcatttttattaaa

SEQ ID NO: 49 - RNA, 20 nt, synthetic construct
  modified_base 1..3: mod_base = OTHER; note = linked via phosphorothioate
  modified_base 1: mod_base = um
  modified_base 2: mod_base = gm
  modified_base 3: mod_base = um
  modified_base 4: mod_base = OTHER; note = 2'-O-methyl-adenosine
  modified_base 5: mod_base = gm
  modified_base 6: mod_base = OTHER; note = 2'-fluoro-cytidine
  modified_base 7: mod_base = OTHER; note = 2'-O-methyl-adenosine
  modified_base 8: mod_base = OTHER; note = 2'-fluoro-uridine
  modified_base 9: mod_base = OTHER; note = 2'-fluoro-uridine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = OTHER; note = 2'-O-methyl-adenosine
  modified_base 14: mod_base = um
  modified_base 15: mod_base = um
  modified_base 16: mod_base = OTHER; note = 2'-O-methyl-adenosine
  modified_base 17: mod_base = OTHER; note = 2'-O-methyl-adenosine
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
tgtagcatttttattaataa

SEQ ID NO: 50 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = um
  modified_base 3: mod_base = um
  modified_base 4: mod_base = cm
  modified_base 5: mod_base = um
  modified_base 6: mod_base = OTHER; note = 2'-fluoro-guanosine
  modified_base 7: mod_base = gm
  modified_base 8: mod_base = OTHER; note = 2'-fluoro-guanosine
  modified_base 9: mod_base = OTHER; note = 2'-fluoro-uridine
  modified_base 10: mod_base = OTHER; note = 2'-fluoro-uridine
  modified_base 11: mod_base = OTHER; note = 2'-fluoro-uridine
  modified_base 12: mod_base = um
  modified_base 13: mod_base = gm
  modified_base 14: mod_base = um
  modified_base 15: mod_base = OTHER; note = 2'-O-methyl-adenosine
  modified_base 16: mod_base = gm
  modified_base 17: mod_base = cm
  modified_base 18..20: mod_base = OTHER; note = linked via phosphorothioate
  modified_base 18: mod_base = OTHER; note = 2'-O-methyl-adenosine
  modified_base 19: mod_base = um
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
attctgggttttgtagcata

SEQ ID NO: 51 - RNA, 20 nt, synthetic construct
  modified_base 1..3: mod_base = OTHER; note = linked via phosphorothioate
  modified_base 1: mod_base = um
  modified_base 2: mod_base = um
  modified_base 3: mod_base = um
  modified_base 4: mod_base = um
  modified_base 5: mod_base = gm
  modified_base 6: mod_base = OTHER; note = 2'-fluoro-uridine
  modified_base 7: mod_base = OTHER; note = 2'-O-methyl-adenosine
  modified_base 8: mod_base = OTHER; note = 2'-fluoro-guanosine
  modified_base 9: mod_base = OTHER; note = 2'-fluoro-cytidine
  modified_base 10: mod_base = OTHER; note = 2'-fluoro-adenosine
  modified_base 11: mod_base = OTHER; note = 2'-fluoro-uridine
  modified_base 12: mod_base = um
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18..20: mod_base = OTHER; note = linked via phosphorothioate
  modified_base 18: mod_base = um
  modified_base 19: mod_base = OTHER; note = 2'-O-methyl-adenosine
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
ttttgtagcatttttattaa

SEQ ID NO: 52 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = OTHER; note = 2'-O-methyl-adenosine
  modified_base 3: mod_base = cm
  modified_base 4: mod_base = um
  modified_base 5: mod_base = um
  modified_base 6: mod_base = OTHER; note = 2'-fluoro-guanosine
  modified_base 7: mod_base = OTHER; note = 2'-O-methyl-adenosine
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-adenosine
  modified_base 11: mod_base = OTHER; note = 2'-fluoro-uridine
  modified_base 12: mod_base = OTHER; note = 2'-O-methyl-adenosine
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18: mod_base = um
  modified_base 19: mod_base = cm
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl
  source 1..20: mol_type = other RNA
aacttgaagatatttattca

SEQ ID NO: 53 - RNA, 22 nt, synthetic construct
  modified_base 1..3: mod_base = OTHER; note = linked via phosphorothioate
  modified_base 1: mod_base = OTHER; note = phosphate mimic linked 2'-O-methyluridine
  modified_base 2: mod_base = OTHER; note = 2'-fluoro-guanosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = um
  modified_base 10: mod_base = OTHER; note = 2'-fluoro-adenosine
  modified_base 11: mod_base = um
  modified_base 12: mod_base = cm
  modified_base 13: mod_base = um
  modified_base 14: mod_base = OTHER; note = 2'-fluoro-uridine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = gm
  modified_base 19: mod_base = um
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = cm
  source 1..22: mol_type = other RNA
tgaataaatatcttcaagttac

SEQ ID NO: 54 - RNA, 22 nt, synthetic construct
  modified_base 1..3: mod_base = OTHER; note = linked via phosphorothioate
  modified_base 1: mod_base = OTHER; note = phosphate mimic linked 2'-O-methyluridine
  modified_base 2: mod_base = OTHER; note = 2'-fluoro-guanosine
  modified_base 3: mod_base = OTHER; note = 2'-fluoro-adenosine
  modified_base 4: mod_base = OTHER; note = 2'-O-methyl-adenosine
  modified_base 5: mod_base = OTHER; note = 2'-fluoro-uridine
  modified_base 6: mod_base = OTHER; note = 2'-O-methyl-adenosine
  modified_base 7: mod_base = OTHER; note = 2'-fluoro-adenosine
  modified_base 8: mod_base = OTHER; note = 2'-O-methyl-adenosine
  modified_base 9: mod_base = um
  modified_base 10: mod_base = OTHER; note = 2'-fluoro-adenosine
  modified_base 11: mod_base = um
  modified_base 12: mod_base = cm
  modified_base 13: mod_base = um
  modified_base 14: mod_base = OTHER; note = 2'-fluoro-uridine
  modified_base 15: mod_base = cm
  modified_base 16: mod_base = OTHER; note = 2'-fluoro-adenosine
  modified_base 17: mod_base = OTHER; note = 2'-O-methyl-adenosine
  modified_base 18: mod_base = gm
  modified_base 19: mod_base = um
  modified_base 20..22: mod_base = OTHER; note = linked via phosphorothioate
  modified_base 20: mod_base = um
  modified_base 21: mod_base = OTHER; note = 2'-O-methyl-adenosine
  modified_base 22: mod_base = cm
  source 1..22: mol_type = other RNA
tgaataaatatcttcaagttac

SEQ ID NO: 55 - RNA, 20 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = OTHER; note = 2'-O-methyl-adenosine
  modified_base 3: mod_base = cm
  modified_base 4: mod_base = um
  modified_base 5: mod_base = um
  modified_base 6: mod_base = OTHER; note = 2'-fluoro-guanosine
  modified_base 7: mod_base = OTHER; note = 2'-O-methyl-adenosine
  modified_base 8: mod_base = OTHER; note = 2'-fluoro-adenosine
  modified_base 9: mod_base = OTHER; note = 2'-fluoro-guanosine
  modified_base 10: mod_base = OTHER; note = 2'-fluoro-adenosine
  modified_base 11: mod_base = OTHER; note = 2'-fluoro-uridine
  modified_base 12: mod_base = OTHER; note = 2'-O-methyl-adenosine
  modified_base 13: mod_base = um
  modified_base 14: mod_base = um
  modified_base 15: mod_base = um
  modified_base 16: mod_base = OTHER; note = 2'-O-methyl-adenosine
  modified_base 17: mod_base = um
  modified_base 18: mod_base = um
  modified_base 19: mod_base = cm
  modified_base 20: mod_base = OTHER; note = 2'-O-methyl-adenosine
  modified_base 20: mod_base = OTHER; note = conjugated to 3 iterations of (2R,3R,4R,5S)-5-(3-(5 -(((2R,3R,4R,5R,6R)-3-acetamido-4,5-dihydroxy-6-(hydroxyme thyl)tetrahydro-2H-pyran-2-yl)oxy)pentanamido)propyl)-2-(( hydroxyl)methyl)-4-methoxytetrahydrofuran-3-oxyl linked via phosphorothioate
  source 1..20: mol_type = other RNA
aacttgaagatatttattca

SEQ ID NO: 56 - DNA, 21 bp, synthetic construct
  source 1..21: mol_type = other DNA
  misc_feature 1..10: note = RNA
  misc_feature 11: note = DNA
  misc_feature 12..21: note = RNA
ctagacctgttttgcttttgt

SEQ ID NO: 57 - RNA, 23 nt, synthetic construct
  source 1..23: mol_type = other RNA
aagatctggacaaaacgaaaaca

(19) United States
(12) Patent Application Publication
(10) Pub. No.: US 2025/0027089 A1
(43) Pub. Date: Jan. 23, 2025

(54) DOUBLE STRANDED RNA TARGETING PROPROTEIN CONVERTASE SUBTILISIN KEXIN 9 (PCSK9) AND METHODS OF USE THEREOF

(71) Applicant: Sanegene Bio USA Inc., Woburn, MA (US)

(72) Inventors: Chunyang ZHANG, Burlington, MA (US); Zhongfa YANG, Sharon, MA (US); Shiyu WANG, Belmont, MA (US); Weimin WANG, Winchester, MA (US); Chunxue ZHOU, Suzhou (CN)

(21) Appl. No.: 18/750,463

(22) Filed: Jun. 21, 2024

(30) Foreign Application Priority Data
Jun. 21, 2023 (WO) PCT/CN2023/101925

(51) Int. Cl. C12N 15/113; A61K 45/06; A61P 3/06

(52) U.S. Cl. CPC C12N 15/1138; A61K 45/06; A61P 3/06; C12N 2310/11; C12N 2310/313; C12N 2310/321; C12N 2310/322; C12N 2310/351; C12Y 304/21061

(57) ABSTRACT

The disclosure relates to isolated oligonucleotides comprising duplex regions targeting Proprotein convertase subtilisin kexin 9 (PCSK9), and delivery systems, kits and compositions comprising same, and methods of using same for inhibiting or downregulating PCSK9.

### RELATED APPLICATIONS

This application claims the priority to, and the benefit of, PCT Application No. PCT/CN2023/101925, filed on Jun. 21, 2023, the entire contents of which are incorporated herein by reference.

### SEQUENCE LISTING

The Sequence Listing XML associated with this application is provided electronically in XML format and is hereby incorporated by reference into the specification. The name of the XML file containing the Sequence Listing XML is “SANB_007_SeqList_ST26.xml”. The XML file is 460,825 bytes in size, created on Jun. 20, 2024, and is being submitted electronically via USPTO Patent Center.

### BACKGROUND

Proprotein convertase subtilisin kexin 9 (PCSK9) is a member of the subtilisin serine protease family and is known to play a role in cholesterol metabolism. Mutations in the PCSK9 gene have been found to be associated with a form of autosomal dominant hypercholesterolemia. PCSK9 has been shown to play a role in controlling hepatic low density lipoprotein receptor (LDLR)-mediated LDL uptake. PCSK9 overexpression studies done in mice showed elevated total and LDL cholesterol levels. PCSK9 overexpression also results in a severe reduction in hepatic LDLR protein associated with increased plasma cholesterol levels, without affecting LDLR mRNA levels, sterol regulatory-element binding proteins (SREBP) protein levels, or SREBP protein nuclear to cytoplasmic ratio. Thus, hepatic PCSK9 levels are positively correlated with circulating LDL-cholesterol levels.

The observed effects of PCSK9 on lipid uptake and metabolism make it an attractive target for therapeutic intervention. Accordingly, there is an unmet need for highly potent and tolerable compounds to inhibit PCSK9. The present application discloses novel, highly potent inhibitors of PCSK9 expression and their use in treatment.

### SUMMARY

The present disclosure provides an isolated oligonucleotide comprising a sense strand and an antisense strand, wherein: the sense strand comprises a nucleotide sequence that is substantially identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region between the nucleotide positions 3543 to 3596, from the 5′ end of a PCSK9 mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is identical to a region between the nucleotide positions 3543 to 3596, from the 5′ end of a PCSK9 mRNA sequence according to SEQ ID NO: 1.

In some embodiments of the isolated oligonucleotide of the present disclosure, the isolated oligonucleotide is capable of inducing degradation of the PCSK9 mRNA.

In some embodiments of the isolated oligonucleotide of the present disclosure, the sense strand is a single stranded RNA molecule. In some embodiments of the isolated oligonucleotide of the present disclosure, the antisense strand is a single stranded RNA molecule. In some embodiments of the isolated oligonucleotide of the present disclosure, both the sense strand and the antisense strand are single stranded RNA molecules.

In some embodiments of the isolated oligonucleotide of the present disclosure, the single stranded RNA molecule of the antisense strand comprises a 3′ overhang. In some embodiments, in the single stranded RNA molecule of the sense strand, the 3′ overhang comprise at least one nucleotide. In some embodiments, in the single stranded RNA molecule of the sense strand, the 3′ overhang comprise two nucleotides.

In some embodiments of the isolated oligonucleotide of the present disclosure, the 3′ overhang comprises any one of thymidine-thymidine (dTdT), Adenine-Adenine (AA), Cysteine-Cysteine (CC), Guanine-Guanine (GG) or Uracil-Uracil (UU).

In some embodiments of the isolated oligonucleotide of the present disclosure, the sense strand comprises an RNA sequence of at least 20 nucleotides in length. In some embodiments of the isolated oligonucleotide of the present disclosure, the sense strand comprises an RNA sequence of 20 nucleotides in length.

In some embodiments of the isolated oligonucleotide of the present disclosure, the antisense strand comprises an RNA sequence of at least 22 nucleotides in length. In some embodiments of the isolated oligonucleotide of the present disclosure, the antisense strand comprises an RNA sequence of 22 nucleotides in length.

In some embodiments of the isolated oligonucleotide of the present disclosure, the double stranded region is between 19 and 21 nucleotides in length. In some embodiments of the isolated oligonucleotide of the present disclosure, the double stranded region is 20 nucleotides in length.

In some embodiments of the isolated oligonucleotide of the present disclosure, the double stranded region comprises an antisense strand and a sense strand, according to any one of the pairs of antisense strand and sense strand sequences in Tables 1-3, as described in the detailed description.

In some embodiments of the isolated oligonucleotide of the present disclosure, the antisense strand comprises a nucleotide sequence according to any one of: SEQ ID NOs: 2-7, 28 or 29.

In some embodiments of the isolated oligonucleotide of the present disclosure, the sense strand comprises a nucleotide sequence according to any one of: SEQ ID NOs: 8-13, 30 or 31.

In some embodiments of the isolated oligonucleotide of the present disclosure, the antisense strand comprises a nucleotide sequence according to any one of: SEQ ID NOs: 2-7, 28 or 29; and the sense strand comprises a nucleotide sequence according to any one of: SEQ ID NOs: 8-13, 30 or 31, wherein the antisense strand and the sense strand sequences have sufficient complementarity to allow formation of a double stranded region between the antisense and the sense strand.

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
iv) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′); or
v) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′).

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises:
i) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
ii) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′); or
iii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

In some embodiments of the isolated oligonucleotide of the present disclosure, the sense strand or the antisense strand or both comprise one or more modified nucleotide(s).

In some embodiments of the isolated oligonucleotide of the present disclosure wherein the sense strand or the antisense strand or both comprise one or more modified nucleotide(s), the antisense strand comprises a mono methyl protected phosphate mimic (5′-MeEP).

In some embodiments of the isolated oligonucleotide of the present disclosure wherein the sense strand or the antisense strand or both comprise one or more modified nucleotide(s), the antisense strand comprises a phosphate mimic linked to a 5′-terminal uracil, wherein the phosphate mimic is selected from:

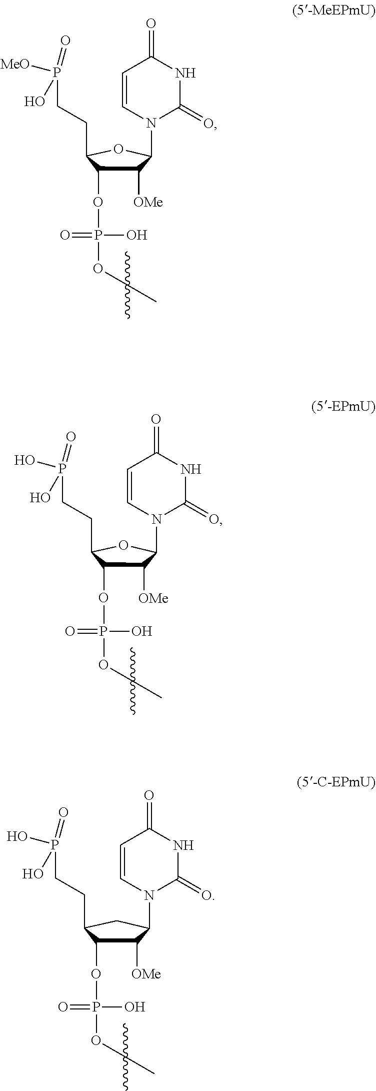

In some embodiments of the isolated oligonucleotide of the present disclosure, in the sense strand or the antisense strand or both, a terminal or internal nucleotide is linked to a targeting ligand.

In some embodiments of the isolated oligonucleotide of the present disclosure, the targeting ligand is attached to the 3′ end (e.g., 3′ terminal position) of the sense strand.

In some embodiments of the isolated oligonucleotide of the present disclosure, the targeting ligand comprises a GalNAc.

In some embodiments of the isolated oligonucleotide of the present disclosure, the targeting ligand comprises at least one GalNAc G1b moiety.

In some embodiments of the isolated oligonucleotide of the present disclosure, the antisense strand comprises nucleotides modified with 2′-F modification, and nucleotides modified with 2′-O-methyl modification, according to the formula:

3′(M)₀(F)₀(M)₆(F)₁(M)₁(F)₁(M)₃(F)₁(M)₂(F)₁(M)₁(F)₁(M)₁(F)₂(M)₁5′.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉3′.

In some embodiments of the isolated oligonucleotide of the present disclosure, the antisense strand comprises any one of:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′);
ii) an antisense strand of nucleic acid sequence according to SEQ ID NO: 19 (5′ [MeEPmUs][fGs][fA][mA][fA][mA][fA][mA][mU][fA][mU][mC][mU][fU][mC][fA][mA][mG][mU][mUs][mAs][mC] 3′);
iii) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′);
viii) an antisense strand of nucleic acid sequence according to SEQ ID NO: 35 (5′ [MeEPmUs][fUs][fA][mA][fU][mA][fA][mA][mA][fA][mU][mG][mC][fU][mA][fC][mA][mA][mA][mAs][mCs][mC] 3′);
ix) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′); or
x) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′),
wherein “m” is a 2′-O-methyl modified nucleotide, “f” is a 2′-F modified nucleotide, “s” is a phosphorothioate internucleotide linkage, “MeEPmU” is

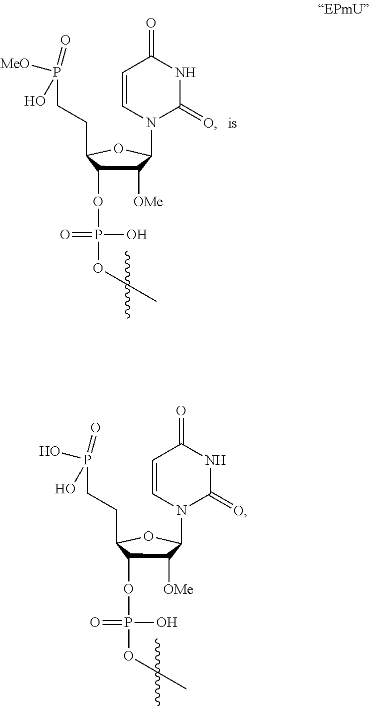

### and “C-EPmU” is

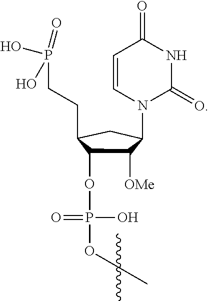

In some embodiments of the isolated oligonucleotide of the present disclosure, the sense strand comprises any one of:
i) a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′); or
ix) a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′),
wherein “m” is a 2′-O-methyl modified nucleotide, “f” is a 2′-F modified nucleotide, “s” is a phosphorothioate internucleotide linkage, and “G1b” is a GalNac G1b moiety.

In some embodiments of the isolated oligonucleotide of the present disclosure, the double stranded region comprises:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′), and
a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) i) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′), and
a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′), and
a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′), and
a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′), and
a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′), and
a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) an antisense strand of nucleic acid sequence according to SEQ ID NO: 35 (5′ [MeEPmUs][fUs][fA][mA][fU][mA][fA][mA][mA][fA][mU][mG][mC][fU][mA][fC][mA][mA][mA][mAs][mCs][mC] 3′), and
a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′);
ix) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′);
x) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′); or
xi) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′),
wherein “m” is a 2′-O-methyl modified nucleotide, “f” is a 2′-F modified nucleotide, “s” is a phosphorothioate internucleotide linkage, “G1b” is a GalNac G1b moiety, “MeEPmU” is

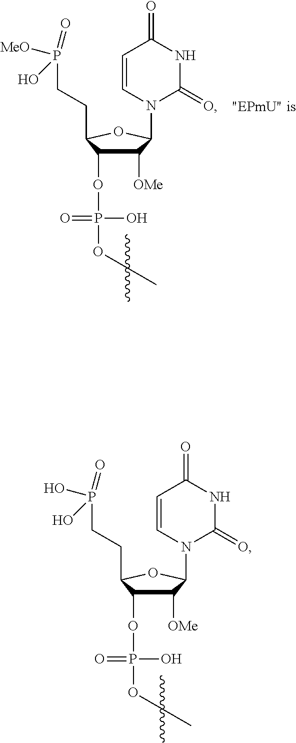

### and “C-EPmU” is

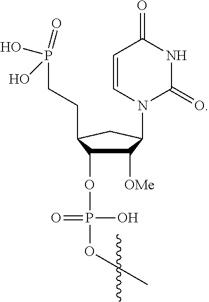

The present disclosure also provides a vector encoding an isolated oligonucleotide disclosed herein.

The present disclosure also provides a delivery system comprising an isolated oligonucleotide or vector disclosed herein.

The present disclosure also provides a pharmaceutical composition comprising an isolated oligonucleotide, vector or delivery system disclosed herein, and a pharmaceutically acceptable carrier, diluent or excipient.

The present disclosure also provides a kit comprising an isolated oligonucleotide, vector, delivery system or a pharmaceutical composition disclosed herein.

The present disclosure also provides a method of inhibiting or downregulating the expression or level of PCSK9 in a subject in need thereof, wherein the method comprises administering to the subject an effective amount of an isolated oligonucleotide, vector, delivery system or a pharmaceutical composition disclosed herein.

In some embodiments of the method of inhibiting or downregulating the expression or level of PCSK9 in a subject in need thereof, the subject has hypercholesterolemia, coronary heart disease, peripheral arterial disease, stroke, type 2 diabetes, obesity, or high blood pressure or a combination thereof.

In some embodiments of the method of inhibiting or downregulating the expression or level of PCSK9 in a subject in need thereof, the method comprises administering the isolated oligonucleotide, the vector, the delivery system, or the pharmaceutical composition, in combination with at least a second therapeutic agent.

The present disclosure also provides a method of inhibiting or downregulating the expression or level of PCSK9 in a subject in need thereof, wherein the method comprises administering to the subject an effective amount of a first and at least a second oligonucleotides disclosed herein, wherein the first and at least second oligonucleotides comprise different sequences.

The present disclosure also provides a method of treating or preventing a disease or disorder associated with aberrant or increased expression or activity of PCSK9 or a disease or disorder where PCSK9 plays a role in a subject in need thereof, wherein the method comprises administering to the subject an effective amount of an isolated oligonucleotide, vector, delivery system or a pharmaceutical composition disclosed herein.

### BRIEF DESCRIPTION OF THE DRAWINGS

FIGS. 1A-1B are a series of graphs depicting in vivo potency of siRNA compounds listed in Table 2 with GalNac conjugations in reducing human PCSK9 mRNA in HDI mouse liver after subcutaneous dosing of siRNA compound at multiple doses, as indicated. FIG. 1A is a graph showing human PCSK9 mRNA remaining in HDI mouse liver using a single dose of 1 mg/kg. FIG. 1B is a graph showing human PCSK9 mRNA remaining in HDI mouse liver using a dose of 0.25 mg/kg (black), 0.5 mg/kg (grey) and 1 mg/kg (white). Data is represented as % of human PCSK9 mRNA remaining relative to PBS groups when normalized to Neomycin-resistant (NeoR) gene mRNA levels (Mean, +/−SD).

FIGS. 2A-2C are a series of graphs depicting in vivo potency of siRNA compounds listed in Table 3 in Macaca fascicularis after a 3 mg/kg single subcutaneous dosing. FIG. 2A shows siRNA potency in in lowering cynomolgus serum LDL-Cholesterol. FIG. 2B shows the siRNA potency in lowering cynomolgus serum PCSK9 proteins. FIG. 2C shows the siRNA effects in lowering cynomolgus liver PCSK9 mRNAs. Data is represented as % remaining relative to PBS groups when normalized to pre-dose levels on day 1 (Mean, +/−SD).

FIGS. 3A-3C are a series of graphs depicting in vivo potency of siRNA compounds listed in Table 3 in Macaca fascicularis after a 3 mg/kg single subcutaneous dosing. FIG. 3A shows siRNA potency in in lowering cynomolgus serum LDL-Cholesterol. FIG. 3B shows the siRNA potency in lowering cynomolgus serum PCSK9 proteins. FIG. 3C shows the siRNA effects in lowering cynomolgus liver PCSK9 mRNAs. Data is represented as % remaining relative to PBS groups when normalized to pre-dose levels on day 1 (Mean, +/−SD).

FIGS. 4A-4B are a series of graphs depicting the ex vivo potency of siRNA compounds listed in Table 3 in silencing human PCSK9 mRNA in primary human hepatocytes (PHH) compared to PBS control. Compounds were directly added to the cultured PHH at multiple doses, as indicated. FIG. 4A shows compounds tested through free-uptake assay at 10 nM. FIG. 4B shows compounds tested through free-uptake assays at 8 doses (0.5 nM, 1 nM, 2 nM, 5 nM, 10 nM, 50 nM, 100 nM, 500 nM). Data is presented as % PCSK9 mRNA remaining when normalized to PBS control (Mean, +/−SD).

FIGS. 5A-5C are a series of graphs depicting the evaluation of the potency of specific siRNA compounds listed in Table 3 in a humanized PCSK9 transgenic mice model fed a regular chow diet, as described herein. FIG. 5A shows the levels of human PSCK9 (hPSCK9) protein in the mice over the course of the study. FIG. 5B shows the levels of LDL-C in the mice over the course of the study. FIG. 5C shows percentage of hPCSK9 mRNA remaining at the conclusion of the study.

FIGS. 6A-6C are a series of graphs depicting the evaluation of the efficacy of siRNA compounds listed in Table 3 in a humanized PCSK9 transgenic mice model fed a high-fat diet, as described herein. FIG. 6A shows the levels of hPSCK9 protein in the mice over the course of the study. FIG. 6B shows the levels of LDL-C in the mice over the course of the study. FIG. 6C shows percentage of hPCSK9 mRNA remaining at the conclusion of the study.

FIGS. 7A-7B are a series of graphs depicting the evaluation of the efficacy siRNA compounds listed in Table 3 in a spontaneous hypercholesterolemia Macaca fascicularis model, as described herein. FIG. 7A shows the percentage of serum PCSK9 protein remaining over the course of the study in monkeys dosed with PBS (control) or the compound of Table 3. FIG. 7B shows the percentage of serum LDL-C remaining over the course of the study in monkeys dosed with PBS (control) or the compound of Table 3.

### DETAILED DESCRIPTION

The present disclosure provides isolated oligonucleotides (oligonucleotide(s)) that form a double stranded region, preferably small interfering RNAs (siRNAs), that can decrease PCSK9 mRNA expression, in turn leading to a decrease in the degree of PCSK9 protein expression in target cells. The oligonucleotides disclosed herein can have therapeutic application in regulating the expression of PCSK9, for treatment of diseases, including but not limited to cardiovascular disease (CVD), atherosclerosis, hypercholesterolemia, hyperlipidemia or hypertriglyceridemia.

The present disclosure has identified specific regions within the PCSK9 mRNA, that provide targets for binding double stranded oligonucleotides, e.g., siRNA, leading to reduction in level of expression of the PCSK9 mRNA.

The PCSK9 mRNA sequence described herein, is an mRNA sequence of PCSK9 according to accession no NM_174936.4:

(SEQ ID NO: 1)
AGCGACGTCGAGGCGCTCATGGTTGCAGGCGGGCGCCGCCGTTCAGTTCAGGGTCT
GAGCCTGGAGGAGTGAGCCAGGCAGTGAGACTGGCTCGGGGGGCCGGGACGCGT
CGTTGCAGCAGCGGCTCCCAGCTCCCAGCCAGGATTCCGCGCGCCCCTTCACGCGCC
CTGCTCCTGAACTTCAGCTCCTGCACAGTCCTCCCCACCGCAAGGCTCAAGGCGCCG
CCGGCGTGGACCGCGCACGGCCTCTAGGTCTCCTCGCCAGGACAGCAACCTCTCCCC
TGGCCCTCATGGGCACCGTCAGCTCCAGGCGGTCCTGGTGGCCGCTGCCACTGCTGC
TGCTGCTGCTGCTGCTCCTGGGTCCCGCGGGCGCCCGTGCGCAGGAGGACGAGGAC
GGCGACTACGAGGAGCTGGTGCTAGCCTTGCGTTCCGAGGAGGACGGCCTGGCCGA
AGCACCCGAGCACGGAACCACAGCCACCTTCCACCGCTGCGCCAAGGATCCGTGGA
GGTTGCCTGGCACCTACGTGGTGGTGCTGAAGGAGGAGACCCACCTCTCGCAGTCA
GAGCGCACTGCCCGCCGCCTGCAGGCCCAGGCTGCCCGCCGGGGATACCTCACCAA
GATCCTGCATGTCTTCCATGGCCTTCTTCCTGGCTTCCTGGTGAAGATGAGTGGCGAC
CTGCTGGAGCTGGCCTTGAAGTTGCCCCATGTCGACTACATCGAGGAGGACTCCTCT
GTCTTTGCCCAGAGCATCCCGTGGAACCTGGAGCGGATTACCCCTCCACGGTACCGG
GCGGATGAATACCAGCCCCCCGACGGAGGCAGCCTGGTGGAGGTGTATCTCCTAGA
CACCAGCATACAGAGTGACCACCGGGAAATCGAGGGCAGGGTCATGGTCACCGACT
TCGAGAATGTGCCCGAGGAGGACGGGACCCGCTTCCACAGACAGGCCAGCAAGTGT
GACAGTCATGGCACCCACCTGGCAGGGGTGGTCAGCGGCCGGGATGCCGGCGTGGC
CAAGGGTGCCAGCATGCGCAGCCTGCGCGTGCTCAACTGCCAAGGGAAGGGCACGG
TTAGCGGCACCCTCATAGGCCTGGAGTTTATTCGGAAAAGCCAGCTGGTCCAGCCTG
TGGGGCCACTGGTGGTGCTGCTGCCCCTGGCGGGTGGGTACAGCCGCGTCCTCAACG
CCGCCTGCCAGCGCCTGGCGAGGGCTGGGGTCGTGCTGGTCACCGCTGCCGGCAAC
TTCCGGGACGATGCCTGCCTCTACTCCCCAGCCTCAGCTCCCGAGGTCATCACAGTT
GGGGCCACCAATGCCCAAGACCAGCCGGTGACCCTGGGGACTTTGGGGACCAACTT
TGGCCGCTGTGTGGACCTCTTTGCCCCAGGGGAGGACATCATTGGTGCCTCCAGCGA
CTGCAGCACCTGCTTTGTGTCACAGAGTGGGACATCACAGGCTGCTGCCCACGTGGC
TGGCATTGCAGCCATGATGCTGTCTGCCGAGCCGGAGCTCACCCTGGCCGAGTTGAG
GCAGAGACTGATCCACTTCTCTGCCAAAGATGTCATCAATGAGGCCTGGTTCCCTGA
GGACCAGCGGGTACTGACCCCCAACCTGGTGGCCGCCCTGCCCCCCAGCACCCATG
GGGCAGGTTGGCAGCTGTTTTGCAGGACTGTATGGTCAGCACACTCGGGGCCTACAC
GGATGGCCACAGCCGTCGCCCGCTGCGCCCCAGATGAGGAGCTGCTGAGCTGCTCC
AGTTTCTCCAGGAGTGGGAAGCGGCGGGGCGAGCGCATGGAGGCCCAAGGGGGCA
AGCTGGTCTGCCGGGCCCACAACGCTTTTGGGGGTGAGGGTGTCTACGCCATTGCCA
GGTGCTGCCTGCTACCCCAGGCCAACTGCAGCGTCCACACAGCTCCACCAGCTGAG
GCCAGCATGGGGACCCGTGTCCACTGCCACCAACAGGGCCACGTCCTCACAGGCTG
CAGCTCCCACTGGGAGGTGGAGGACCTTGGCACCCACAAGCCGCCTGTGCTGAGGC
CACGAGGTCAGCCCAACCAGTGCGTGGGCCACAGGGAGGCCAGCATCCACGCTTCC
TGCTGCCATGCCCCAGGTCTGGAATGCAAAGTCAAGGAGCATGGAATCCCGGCCCC
TCAGGAGCAGGTGACCGTGGCCTGCGAGGAGGGCTGGACCCTGACTGGCTGCAGTG
CCCTCCCTGGGACCTCCCACGTCCTGGGGGCCTACGCCGTAGACAACACGTGTGTAG
TCAGGAGCCGGGACGTCAGCACTACAGGCAGCACCAGCGAAGGGGCCGTGACAGC
CGTTGCCATCTGCTGCCGGAGCCGGCACCTGGCGCAGGCCTCCCAGGAGCTCCAGTG
ACAGCCCCATCCCAGGATGGGTGTCTGGGGAGGGTCAAGGGCTGGGGCTGAGCTTT
AAAATGGTTCCGACTTGTCCCTCTCTCAGCCCTCCATGGCCTGGCACGAGGGGATGG
GGATGCTTCCGCCTTTCCGGGGCTGCTGGCCTGGCCCTTGAGTGGGGCAGCCTCCTT
GCCTGGAACTCACTCACTCTGGGTGCCTCCTCCCCAGGTGGAGGTGCCAGGAAGCTC
CCTCCCTCACTGTGGGGCATTTCACCATTCAAACAGGTCGAGCTGTGCTCGGGTGCT
GCCAGCTGCTCCCAATGTGCCGATGTCCGTGGGCAGAATGACTTTTATTGAGCTCTT
GTTCCGTGCCAGGCATTCAATCCTCAGGTCTCCACCAAGGAGGCAGGATTCTTCCCA
TGGATAGGGGAGGGGGCGGTAGGGGCTGCAGGGACAAACATCGTTGGGGGGTGAG
TGTGAAAGGTGCTGATGGCCCTCATCTCCAGCTAACTGTGGAGAAGCCCCTGGGGGC
TCCCTGATTAATGGAGGCTTAGCTTTCTGGATGGCATCTAGCCAGAGGCTGGAGACA
GGTGCGCCCCTGGTGGTCACAGGCTGTGCCTTGGTTTCCTGAGCCACCTTTACTCTGC
TCTATGCCAGGCTGTGCTAGCAACACCCAAAGGTGGCCTGCGGGGAGCCATCACCT
AGGACTGACTCGGCAGTGTGCAGTGGTGCATGCACTGTCTCAGCCAACCCGCTCCAC
TACCCGGCAGGGTACACATTCGCACCCCTACTTCACAGAGGAAGAAACCTGGAACC
AGAGGGGGCGTGCCTGCCAAGCTCACACAGCAGGAACTGAGCCAGAAACGCAGATT
GGGCTGGCTCTGAAGCCAAGCCTCTTCTTACTTCACCCGGCTGGGCTCCTCATTTTTA
CGGGTAACAGTGAGGCTGGGAAGGGGAACACAGACCAGGAAGCTCGGTGAGTGAT
GGCAGAACGATGCCTGCAGGCATGGAACTTTTTCCGTTATCACCCAGGCCTGATTCA
CTGGCCTGGCGGAGATGCTTCTAAGGCATGGTCGGGGGAGAGGGCCAACAACTGTC
CCTCCTTGAGCACCAGCCCCACCCAAGCAAGCAGACATTTATCTTTTGGGTCTGTCC
TCTCTGTTGCCTTTTTACAGCCAACTTTTCTAGACCTGTTTTGCTTTTGTAACTTGAAG
ATATTTATTCTGGGTTTTGTAGCATTTTTATTAATATGGTGACTTTTTAAAATAAAAA
CAAACAAACGTTGTCCTAA.

The present disclosure provides an isolated oligonucleotide comprising a sense strand and an antisense strand, wherein the sense strand comprises a nucleotide sequence that is substantially identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1.

In some embodiments of the isolated oligonucleotide of the present disclosure, the sense strand comprises a nucleotide sequence that is substantially identical to a region between the nucleotide positions 3569 to 3593, from the 5′ end of a PCSK9 mRNA sequence according to SEQ ID NO: 1, from the 5′ end of an PCSK9 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between the nucleotide positions 3569 to 3593, from the 5′ end of a PCSK9 mRNA sequence according to SEQ ID NO: 1.

In some embodiments of the isolated oligonucleotide of the present disclosure, the sense strand comprises a nucleotide sequence that is identical to a region between the nucleotide positions 3569 to 3593, from the 5′ end of a PCSK9 mRNA sequence according to SEQ ID NO: 1.

The PCSK9 mRNA sequence according to SEQ ID NO: 1, as described herein, is any heterologous mRNA sequence with sufficient identity to an PCSK9 according to accession no. NM_174936.4, as described herein, that allows binding to the sense strand of the oligonucleotides of the present disclosure.

In some embodiments of the isolated oligonucleotide of the present disclosure, the isolated oligonucleotide is capable of inducing degradation of the PCSK9 mRNA.

In some embodiments of the isolated oligonucleotide of the present disclosure, the sense strand is a single stranded RNA molecule. In some embodiments of the isolated oligonucleotide of the present disclosure, the anti-sense strand is a single stranded RNA molecule. In some embodiments of the isolated oligonucleotide of the present disclosure, both the sense strand and the anti-sense strand are single stranded RNA molecules.

In some embodiments, the isolated oligonucleotide of the present disclosure is a small interfering RNA (siRNA). Accordingly, the disclosure provides siRNAs, wherein the siRNA comprises a sense region and anti-sense region complementary to the sense region that together form an RNA duplex, and wherein the sense region comprises a sequence at least 70/to 100% identical to a PCSK9 mRNA sequence.

### Definitions

“RNAi” or “RNA interference” refers to the process of sequence-specific post-transcriptional gene silencing, mediated by double-stranded RNA (dsRNA). Duplex RNA siRNA (small interfering RNA), miRNA (micro RNA), shRNA (short hairpin RNA), ddRNA (DNA-directed RNA), pi RNA (Piwi-interacting RNA), or rasiRNA (repeat associated siRNA) and modified forms thereof are all capable of mediating RNA interference. These dsRNA molecules may be commercially available or may be designed and prepared based on known sequence information, etc. The antisense strand of these molecules can include RNA, DNA, PNA, or a combination thereof. These DNA/RNA chimera polynucleotide includes, but is not limited to, a double-strand polynucleotide composed of DNA and RNA that inhibits the expression of a target gene. These dsRNA molecules can also include one or more modified nucleotides, as described herein, which can be incorporated on either strand.

In the RNAi gene silencing or knockdown process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, decrease messenger RNA of target gene, leading to a phenotype that may come to closely resemble the phenotype arising from a complete or partial deletion of the target gene.

Certain dsRNAs in cells can undergo the action of Dicer enzyme, a ribonuclease III enzyme. Dicer can process the dsRNA into shorter pieces of dsRNA, i.e. siRNAs. RNAi also involves an endonuclease complex known as the RNA induced silencing complex (RISC). Following cleavage by Dicer, siRNAs enter the RISC complex and direct cleavage of a single stranded RNA target having a sequence complementary to the antisense strand of the siRNA duplex. The other strand of the siRNA is the passenger strand. Cleavage of the target RNA takes place in the middle of the region complementary to the antisense strand of the siRNA duplex. siRNAs can thus down regulate or knock down gene expression by mediating RNA interference in a sequence-specific manner.

As used herein, “target gene” or “target sequence” refers to a gene or gene sequence whose corresponding RNA is targeted for degradation through the RNAi pathway using dsRNAs or siRNAs as described herein. To target a gene, for example using an siRNA, the siRNA comprises an antisense region complementary to, or substantially complementary to, at least a portion of the target gene or sequence, and sense strand complementary to the antisense strand. Once introduced into a cell, the siRNA directs the RISC complex to cleave an RNA comprising a target sequence, thereby degrading the RNA.

As used herein, “oligonucleotide”, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide of this disclosure. When dsRNA is produced synthetically, less common bases, such as inosine, 5-methylcytosine, 6-methyladenine, hypoxanthine and others can also be used for antisense, dsRNA, and ribozyme pairing. Other modifications, such as modification to the phosphodiester backbone, or the 2′-fluoro, the 2′-hydroxy or 2′O-methyl in the ribose sugar group of the RNA can also be made.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “region” or “fragment” is used interchangeably and as applied to an oligonucleotide.

The PCSK9 mRNA sequence, as described herein, will be understood to mean a full length PCSK9 mRNA nucleotide sequence, unless indicated otherwise. In some embodiments, the PCSK9 mRNA sequence can be a nucleotide sequence of reduced length relative to a reference nucleic acid or a nucleotide sequence of the PCSK9 mRNA sequence comprising, consisting essentially of, and/or consisting of a nucleotide sequence of contiguous nucleotides identical or almost identical (e.g., 60%, 70%, 80%, 90%, 92%, 95%, 98% or 99% identical) to the reference nucleic acid or nucleotide sequence. Such a nucleic acid fragment according to the disclosure may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the disclosure.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the term “substantially complementary” is at least 90% (e.g., 91%, 92%, 93%, 94%, 95%, 96%, 97%, 98% or 99%) complementary to the sense strand that is substantially identical to the nucleotide sequence within the defined regions in SEQ ID NO: 1. As used herein, the term “substantially complementary” means that two nucleic acid sequences are complementary at least at 90%, 95% or 99% of their nucleotides.

In some embodiments, the two nucleic acid sequences can be complementary at least at 90%, 95%, 96%, 97%, 98%, 99% or more of their nucleotides. In some embodiments, the two nucleic acid sequences can be between 90% to 95% complementary, between 70% to 100% complementary, between 95% and 96% complementary, between 90% and 100% complementary, between 96% to 97% complementary, between 60%6 to 80% complementary, between 97% and 98% complementary, between 70% and 90% complementary, between 98% and 99% complementary, between 80% and 100% complementary, or between 99% and 100% complementary.

The term “substantially complementary” can also mean that two nucleic acid sequences, sense strand and antisense strand have sufficient complementarity that allows binding between the sense strand and antisense strand to form a double stranded region comprising of between 19-25 nucleotides in length. The term “substantially complementary” can also mean that two nucleic acid sequences can hybridize under high stringency conditions, and such conditions are well known in the art.

As used herein, the term “substantially identical” or “sufficient identity” used interchangeably herein, is at least 70%, at least 80%, at least 90%, at least 95%, or at least 99% (e.g., between 70% to 80%, 80% to 90% or 90% to 95% or 95% to 99% or 99% to 100%) identical to the nucleotide sequence within the defined regions in SEQ ID NO: 1.

“Nucleotide sequence” and “nucleic acid sequence” are used interchangeably herein, unless indicated otherwise.

As used herein, the term “identity” means that sequences are compared with one another as follows. In order to determine the percentage identity of two nucleic acid sequences, the sequences can first be aligned with respect to one another in order subsequently to make a comparison of these sequences possible. For this e.g., gaps can be inserted into the sequence of the first nucleic acid sequence and the nucleotides can be compared with the corresponding position of the second nucleic acid sequence. If a position in the first nucleic acid sequence is occupied by the same nucleotide as is the case at a position in the second sequence, the two sequences are identical at this position. The percentage identity between two sequences is a function of the number of identical positions divided by the number of all the positions compared in the sequences investigated.

A “percent identity” or “% identity” as used interchangeably herein, for aligned segments of a test sequence and a reference sequence is the percent of identical components which are shared by the two aligned sequences divided by the total number of components in reference sequence segment, i.e., the entire reference sequence or a smaller defined part of the reference sequence.

The percentage identity of two sequences can be determined with the aid of a mathematical algorithm. A preferred, but not limiting, example of a mathematical algorithm which can be used for comparison of two sequences is the algorithm of Karlin et al. (1993), PNAS USA, 90:5873-5877. Such an algorithm is integrated in the NBLAST program, with which sequences which have a desired identity to the sequences of the present disclosure can be identified. In order to obtain a gapped alignment, as described here, the “Gapped BLAST” program can be used, as is described in Altschul et al. (1997), Nucleic Acids Res, 25:3389-3402. If BLAST and Gapped BLAST programs are used, the preset parameters of the particular program (e.g. NBLAST) can be used. The sequences can be aligned further using version 9 of GAP (global alignment program) of the “Genetic Computing Group” using the preset (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of agreements as a percentage content of the nucleic acids in the sequence claimed. The methods described for determination of the percentage identity of two nucleic acid sequences can also be used correspondingly, if necessary, on the coded amino acid sequences.

Useful methods for determining sequence identity are also disclosed in Guide to Huge Computers (Martin J. Bishop, ed., Academic Press, San Diego (1994)), and Carillo, H., and Lipton, D., (Applied Math 48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Percent identity can be 70% identity or greater, e.g., at least 70% identity, at least 75% identity, at least 80% identity, at least 85% identity, at least 90% identity, at least 95% identity, at least 98% identity, at least 99% identity or 100% identity.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

### Double Stranded RNAs Targeting PCSK9

The disclosure provides isolated oligonucleotides comprising a double stranded RNAs (dsRNAs) duplex region which target a PCSK9 mRNA sequence for degradation. The double stranded RNA molecule of the disclosure may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the dsRNA is a small interfering RNA (siRNA) which targets a PCSK9 mRNA sequence for degradation. In some embodiments, the siRNA targeting PCSK9 is packaged in a delivery system described herein (e.g., nanoparticle).

The isolated oligonucleotides of the present disclosure targeting PCSK9 for degradation can comprise a sense strand at least 70% identical to any fragment of a PCSK9 mRNA, for example the PCSK9 mRNA of SEQ ID NO: 1. In some embodiments, the sense strand comprises or consists essentially of a sequence at least 70%, at least 80%, at least 90%, at least 95% or is 100% identical to any fragment of SEQ ID NO: 1. The siRNAs targeting PCSK9 for degradation can comprise an antisense strand at least 70% identical to a sequence complementary to any fragment of a PCSK9 mRNA, for example the PCSK9 mRNA of SEQ ID NO: 1. In some embodiments, the antisense strand comprises or consists essentially of a sequence at least 70%, at least 80%, at least 90%, at least 95% or is 100% identical to a sequence complementary to any fragment of SEQ ID NO: 1. In some embodiments, the sense region and antisense regions are complementary, and base pair to form an RNA duplex structure. The fragment of the PCSK9 mRNA that has percent identity to the sense region of the siRNA, and which is complementary to the antisense region of the siRNA, can be protein coding sequence of the mRNA, an untranslated region (UTR) of the mRNA (5′ UTR or 3′ UTR), or both.

In some embodiments, the isolated oligonucleotides of the present disclosure comprises a sense region and antisense region complementary to the sense region that together form an RNA duplex, and the sense region comprises a sequence at least 70% identical to a PCSK9 mRNA sequence. In some embodiments, the sense region is identical to a PCSK9 mRNA sequence.

As used herein, the term “sense strand” or “sense region” refers to a nucleotide sequence of an siRNA molecule that is partially or fully complementary to at least a portion of a corresponding antisense strand or antisense region of the siRNA molecule. The sense strand of an isolated oligonucleotides of the present disclosure molecule can include a nucleic acid sequence having some percentage identity with a target nucleic acid sequence such as a PCSK9 mRNA sequence. In some cases, the sense region may have 100% identity, i.e. complete identity or homology, to the target nucleic acid sequence. In other cases, there may be one or more mismatches between the sense region and the target nucleic acid sequence. For example, there may be 1, 2, 3, 4, 5, 6, or 7 mismatches between the sense region and the target nucleic acid sequence.

As used herein, the term “antisense strand” or “antisense region” refers to a nucleotide sequence of the isolated oligonucleotides of the present disclosure, that is partially or fully complementary to at least a portion of a target nucleic acid sequence. The antisense strand of an isolated oligonucleotides of the present disclosure molecule can include a nucleic acid sequence that is complementary to at least a portion of a corresponding sense strand of the isolated oligonucleotides.

In some embodiments, the sense region comprises a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region consists essentially of a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region comprises a sequence that is identical to a sequence of SEQ ID NO: 1 or a region of SEQ ID NO: 1, as disclosed herein. In some embodiments, the sense region consists essentially of a sequence that is identical to a sequence of SEQ ID NO: 1 or a region of SEQ ID NO: 1, as disclosed herein.

In some embodiments, the sense region of the isolated oligonucleotides of the present disclosure targeting PCSK9 has one or more mismatches between the sequence of the isolated oligonucleotides and the PCSK9 sequence. For example, the sequence of the sense region may have 1, 2, 3, 4 or 5 mismatches between the sequence of the sense region of the isolated oligonucleotides and the PCSK9 sequence. In some embodiments, the PCSK9 sequence is an PCSK9 3′ untranslated region sequence (3′ UTR). Without wishing to be bound by theory, it is thought that siRNAs targeting the 3′ UTR have elevated mismatch tolerance when compared to mismatches in the isolated oligonucleotides targeting coding regions of a gene. Further, the isolated oligonucleotides RNAs may be tolerant of mismatches outside the seed region. As used herein, the “seed region” of the isolated oligonucleotides refers to base pairs 2-8 of the antisense region of the isolated oligonucleotides, i.e., the strand of the isolated oligonucleotides that is complementary to and hybridizes to the target mRNA.

In some embodiments, the antisense region comprises a sequence that is at least 70% identical, at least 75% identical, at least 80% identical, at least 85% identical, at least 90% identical, at least 95% identical, at least 97% identical, at least 99% identical or 100% identical to a sequence complementary to a sequence of SEQ ID NO: 1 or a region of SEQ ID NO: 1, as disclosed herein. In some embodiments, the antisense region consists essentially of a sequence that is at least 70% identical, at least 75% identical, at least 80% identical, at least 85% identical, at least 90% identical, at least 95% identical, at least 97% identical, at least 99% or 100% identical to a sequence complementary to a sequence of SEQ ID NO: 1 or a region of SEQ ID NO: 1. In some embodiments, the antisense region comprises a sequence that is identical to a sequence complementary to a sequence of SEQ ID NO: 1 or a region of SEQ ID NO: 1. In some embodiments, the sense region consists essentially of a sequence that is complementary to a sequence of SEQ ID NO: 1 or a region of SEQ ID NO: 1.

The antisense region of the PCSK9 targeting isolated oligonucleotide of the present disclosure is complementary to the sense region. In some embodiments, the sense region and the antisense region are fully complementary (no mismatches). In some embodiments the antisense region is partially complementary to the sense region, i.e., there are 1, 2, 3, 4 or 5 mismatches between the sense region and the antisense region.

In general, isolated oligonucleotide of the present disclosure comprise an RNA duplex that is about 16 to about 25 nucleotides in length. In some embodiments, the RNA duplex is between about 17 and about 24 nucleotides in length, between about 18 and about 23 nucleotides in length, or between about 19 and about 22 nucleotides in length. In some embodiments, the RNA duplex is 19 nucleotides in length. In some embodiments, the RNA duplex is 20 nucleotides in length.

In some embodiments of the isolated oligonucleotide of the present disclosure, the sense strand is a single stranded RNA molecule. In some embodiments of the isolated oligonucleotide of the present disclosure, the antisense strand is a single stranded RNA molecule. In some embodiments of the isolated oligonucleotide of the present disclosure, both the sense strand and the antisense strand are single stranded RNA molecules. In some embodiments of the isolated oligonucleotide of the present disclosure is an siRNA targeting PCSK9, that comprises two different single stranded RNAs, the first comprising the sense region and the second comprising the antisense region, which hybridize to form an RNA duplex.

In some embodiments, the isolated oligonucleotide of the present disclosure, can have one or more overhangs from the duplex region. The overhangs, which are non-base-paired, single strand regions, can be from one to eight nucleotides in length, or longer. An overhang can be a 3′ overhang, wherein the 3′-end of a strand has a single strand region of from one to eight nucleotides in length. An overhang can be a 5′ overhang, wherein the 5′-end of a strand has a single strand region of from one to eight nucleotides in length.

The overhangs of the isolated oligonucleotide of the present disclosure can be the same length or can be different lengths.

In some embodiments of the isolated oligonucleotide of the present disclosure, the single stranded RNA molecule of the sense strand comprises a 3′ overhang. In some embodiments, in the single stranded RNA molecule of the sense strand, the 3′ overhang comprise at least one nucleotide. In some embodiments, in the single stranded RNA molecule of the sense strand, the 3′ overhang comprise two nucleotides.

In some embodiments of the isolated oligonucleotide of the present disclosure, the single stranded RNA molecule of the antisense strand comprises a 3′ overhang. In some embodiments, in the single stranded RNA molecule of the antisense strand, the 3′ overhang comprise at least one nucleotide. In some embodiments, in the single stranded RNA molecule of the antisense strand, the 3′ overhang comprise two nucleotides.

In additional embodiments, both ends of isolated oligonucleotide of the present disclosure have an overhang, for example, a 3′ dinucleotide overhang on each end. The overhangs at the 5′- and 3′-ends may be of different lengths or be the same length.

An overhang of an isolated oligonucleotide of the present disclosure can contain one or more deoxyribonucleotides, one or more ribonucleotides, or a combination of deoxyribonucleotides and ribonucleotides. In some embodiments, one, or both, of the overhang nucleotides of an siRNA may be 2′-deoxyribonucleotides.

In some embodiments, the first single stranded RNA molecule comprises a first 3′ overhang. In some embodiments, the second single stranded RNA molecule comprises a second 3′ overhang. In some embodiments, the first and second 3′ overhangs comprise a dinucleotide.

In some embodiments of the isolated oligonucleotide of the present disclosure, the 3′ overhang comprises any one of thymidine-thymidine (dTdT), Adenine-Adenine (AA), Cysteine-Cysteine (CC), Guanine-Guanine (GG) or Uracil-Uracil (UU). In some embodiments, the isolated oligonucleotide of the present disclosure, the 3′ overhang comprises a thymidine-thymidine (dTdT) or a Uracil-Uracil (UU) overhang. In some embodiment, the 3′ overhang comprises a Uracil-Uracil (UU) overhang. Without wishing to be bound by theory, it is thought that 3′ overhangs, such as dinucleotide overhangs, enhance siRNA mediated mRNA degradation by enhancing siRNA-RISC complex formation, and/or rate of cleavage of the target mRNA by the siRNA-RISC complex.

In some embodiments, the isolated oligonucleotide of the present disclosure can have one or more blunt ends, in which the duplex region ends with no overhang, and the strands are base paired to the end of the duplex region. In some embodiments, the isolated oligonucleotide of the present disclosure can have one or more blunt ends, or can have one or more overhangs, or can have a combination of a blunt end and an overhang end. For example, the 5′ end of the siRNA can be blunt and the 3′ end of the same isolated oligonucleotide comprise an overhang, or vice versa.

In some embodiments, both ends of the isolated oligonucleotide of the present disclosure are blunt ends.

In some embodiments of the isolated oligonucleotide of the present disclosure, the double stranded region comprises an antisense strand and a sense strand, according to any one of the pairs of antisense strand and sense strand sequences in Table 1, as described below.

TABLE 1
Exemplary Sequences of the Present Application

| | | | | Passenger | | | |
|---|---|---|---|---|---|---|---|
| Guide strand | SEQ | Guide | Guide | strand | SEQ | | |
| sequence (5′- | ID | 3′ | 5′ | sequence (5′- | ID | Passenger | Passenger |
| 3′) | NO | position | position | 3′) | NO | 5′position | 3′position |
| UUAUCUUC | 2 | 3543 | 3563 | CUUUUGUAA | 8 | 3545 | 3563 |
| AAGUUACA | | | | CUUGAAGAU | | | |
| AAAGCA | | | | AA | | | |
| UGAAUAAA | 3 | 3550 | 3570 | AACUUGAAG | 9 | 3552 | 3570 |
| UAUCUUCA | | | | AUAUUUAUU | | | |
| AGUUAC | | | | CA | | | |
| UAAAAAUG | 4 | 3569 | 3589 | UGGGUUUUG | 10 | 3571 | 3589 |
| CUACAAAA | | | | UAGCAUUUU | | | |
| CCCAGA | | | | UA | | | |
| UAAUAAAA | 5 | 3572 | 3592 | GUUUUGUAG | 11 | 3574 | 3592 |
| AUGCUACA | | | | CAUUUUUAU | | | |
| AAACCC | | | | UA | | | |
| UUUAAUAA | 6 | 3574 | 3594 | UUUGUAGCA | 12 | 3576 | 3594 |
| AAAUGCUA | | | | UUUUUAUUA | | | |
| CAAAAC | | | | AA | | | |
| UUAUUAAU | 7 | 3576 | 3596 | UGUAGCAUU | 13 | 3578 | 3596 |
| AAAAAUGC | | | | UUUAUUAAU | | | |
| UACAAA | | | | AA | | | |
| UAUGCUAC | 28 | 3565 | 3585 | AUUCUGGGU | 30 | 3567 | 3585 |
| AAAACCCA | | | | UUUGUAGCA | | | |
| GAAUAA | | | | UA | | | |
| UUAAUAAA | 29 | 3573 | 3593 | UUUUGUAGC | 31 | 3575 | 3593 |
| AAUGCUAC | | | | AUUUUUAUU | | | |
| AAAACC | | | | AA | | | |

In some embodiments, the sense region comprises a sequence selected from any one of the group of sense strand/passenger strand sequences listed in Tables 1-3. In some embodiments, the antisense region comprises a sequence selected from any one of the group of antisense strand/guide strand sequences listed in Tables 1-3. In some embodiments, the sense and antisense regions comprise complementary sequences selected from the group listed in Tables 1-3.

In some embodiments of the isolated oligonucleotide of the present disclosure, the antisense strand comprises a nucleotide sequence according to any one of: SEQ ID NOs: 2-7, 28 or 29.

In some embodiments of the isolated oligonucleotide of the present disclosure, the sense strand comprises a nucleotide sequence according to any one of: SEQ ID NOs: 8-13, 30 or 31.

In some embodiments of the isolated oligonucleotide of the present disclosure, the antisense strand comprises a nucleotide sequence according to any one of: SEQ ID NOs: 2-7, 28 or 29; and the sense strand comprises a nucleotide sequence according to any one of: SEQ ID NOs: 8-13, 30 or 31, wherein the antisense strand and the sense strand sequences have sufficient complementarity to allow formation of a double stranded region between the antisense and the sense strand.

In some embodiments of the isolated oligonucleotide of the present disclosure, the isolated oligonucleotide comprises: (a) a sense strand comprising X1 nucleotides, wherein at least one nucleotide is modified with a first modification, each of the remaining nucleotides is independently modified with a second modification, and X1 is an integer selected from 13-36, wherein the first modification and the second modification are different; and (b) an antisense strand comprising X2 nucleotides, wherein at least one nucleotide is modified with a third modification, each of the remaining nucleotides is independently modified with a fourth modification, and X2 is an integer selected from 18-31, wherein the third modification and the fourth modification are different.

In some embodiments, the X1 nucleotides of the sense strand of the isolated oligonucleotide of the present disclosure is 18-21 and the X2 nucleotides of the antisense strand of the isolated oligonucleotide of the present disclosure is 20-23. In some embodiments, the X1 nucleotides of the sense strand of the isolated oligonucleotide of the present disclosure is 20 or 21 and the X2 nucleotides of the antisense strand of the isolated oligonucleotide of the present disclosure is 22 or 23. In some embodiments, the X2 nucleotides of the antisense strand of the isolated oligonucleotide of the present disclosure equals the X1 nucleotides of the sense strand of the isolated oligonucleotide of the present disclosure plus 2. In some embodiments, the X1 nucleotides of the sense strand of the isolated oligonucleotide of the present disclosure is 21 and the X2 nucleotides of the antisense strand of the isolated oligonucleotide of the present disclosure is 23. In some embodiments, the X1 nucleotides of the sense strand of the isolated oligonucleotide of the present disclosure is 20 and the X2 nucleotides of the antisense strand of the isolated oligonucleotide of the present disclosure is 22.

In some embodiments of the isolated oligonucleotide of the present disclosure, the isolated oligonucleotide comprises: (a) a sense strand comprising 20 nucleotides, wherein at least one nucleotide is modified with a first modification, each of the remaining nucleotides is independently modified with a second modification, wherein the first modification and the second modification are the same or different; and (b) an antisense strand comprising 22 nucleotides, wherein at least one nucleotide is modified with a third modification, each of the remaining nucleotides is independently modified with a fourth modification, wherein the third modification and the fourth modification are the same or different.

In some embodiments, the sense strand of the isolated oligonucleotide of the present disclosure comprises at least one nucleotide having a modified phosphate backbone. In some embodiments, the antisense strand of the isolated oligonucleotide of the present disclosure comprises at least one nucleotide having a modified phosphate backbone. In some embodiments, in the sense strand or the antisense strand or both sense and antisense strands of the isolated oligonucleotide of the present disclosure, the modified phosphate backbone comprises a modified phosphodiester bond. In some embodiments, the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage. In some embodiments, the modified phosphodiester bond comprises phosphorothioate, phosphorodithioate, methylphosphonate, phosphoramidate diester, mesyl phosphoramidate, or phosphonoacetate.

In some embodiments, the isolated oligonucleotide of the present disclosure comprises one or more non-natural base-containing nucleotide, a locked nucleotide, or an abasic nucleotide. In some embodiments, the isolated oligonucleotide of the present disclosure, the terminal nucleotide at the 5′ end comprises a phosphate mimic. In some embodiments, the 5′-phosphate mimic is ethylphosphonate, vinylphosphonate or an analog thereof.

In some embodiments, the antisense strand of the isolated oligonucleotide of the present disclosure comprises at least two single-stranded nucleotides at the 3′-terminus. In some embodiments, the antisense strand of the isolated oligonucleotide of the present disclosure comprises two single-stranded nucleotides at the 3′-terminus.

The present disclosure provides an isolated oligonucleotide comprising a sense strand and an antisense strand, wherein the sense strand comprises a nucleotide sequence that is substantially identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is at least 70%, at least 80%, at least 90%, at least 95%, or at least 99% identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region.

In some embodiments of the isolated oligonucleotide of the present disclosure, the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1.

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
iv) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′); or
v) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′).

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises:
i) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
ii) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′); or
iii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

In some embodiments of the isolated oligonucleotide of the present disclosure, the sense strand comprises a nucleotide sequence that is substantially identical to a region between the nucleotide positions 3569 to 3593, from the 5′ end of a PCSK9 mRNA sequence according to SEQ ID NO: 1, from the 5′ end of an PCSK9 mRNA sequence according to SEQ ID NO: 1.

In some embodiments of the isolated oligonucleotide of the present disclosure, the sense strand comprises a nucleotide sequence that is at least 70%, at least 80%, at least 90%, at least 95%, or at least 99% identical to a region between the nucleotide positions 3569 to 3593, from the 5′ end of a PCSK9 mRNA sequence according to SEQ ID NO: 1.

In some embodiments of the isolated oligonucleotide of the present disclosure, the sense strand comprises a nucleotide sequence that is identical to a region between the nucleotide positions 3569 to 3593, from the 5′ end of a PCSK9 mRNA sequence according to SEQ ID NO: 1.

In some embodiments of the isolated oligonucleotide of the present disclosure, wherein the sense strand comprises a nucleotide sequence that is identical to a region between the nucleotide positions 3569 to 3593, from the 5′ end of a PCSK9 mRNA sequence according to SEQ ID NO: 1, the double stranded region comprises:
i) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
ii) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′); or
iii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

### At Least 50% at 0.02 nM

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, the isolated oligonucleotide attenuates expression of the PCSK9 mRNA by 20% to 50% (e.g., between 20% to 25%, 25% to 30%, 30% to 35%, 35% to 40%, 40% to 45% or 45% to 50%), at a dose 0.02 nM.

In some embodiments of the isolated oligonucleotide of the present disclosure wherein the sense strand comprises a nucleotide that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region and that attenuates expression of the PCSK9 mRNA by 20% to 50%, at a dose of 0.02 nM, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
iv) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′);
v) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
vi) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′);
vii) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′); or
viii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

### At Least 50% at 0.1 nM

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, the isolated oligonucleotide attenuates expression of the PCSK9 mRNA by 20% to 50% (e.g., between 20% to 25%, 25% to 30%, 30% to 35%, 35% to 40%, 40% to 45% or 45% to 50%), at a dose 0.1 nM.

In some embodiments of the isolated oligonucleotide of the present disclosure wherein the sense strand comprises a nucleotide that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region and that attenuates expression of the PCSK9 mRNA by 20% to 50%, at a dose of 0.1 nM, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
iv) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′);
v) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
vi) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′);
vii) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′); or
viii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

### At Least 50% at 0.5 nM

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, the isolated oligonucleotide attenuates expression of the PCSK9 mRNA by 20% to 50% (e.g., between 20% to 25%, 25% to 30%, 30% to 35%, 35% to 40%, 40% to 45% or 45% to 50%), at a dose 0.5 nM.

In some embodiments of the isolated oligonucleotide of the present disclosure wherein the sense strand comprises a nucleotide that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region and that attenuates expression of the PCSK9 mRNA by 20% to 50%, at a dose of 0.5 nM, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′),
iv) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′);
v) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
vi) an antisense strand of nucleic acid sequence according to SEQ ID NO. 7 (5′ UUAUUAAUAAAAAUGCUACAAA 3′), and a sense strand of nucleic acid sequence according to SEQ ID NO: 13 (5′ UGUAGCAUUUUUAUUAAUAA 3′);
vii) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′); or
viii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

### At Least 50% at 1.5 nM

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, the isolated oligonucleotide attenuates expression of the PCSK9 mRNA by 20% to 50% (e.g., between 20% to 25%, 25% to 30%, 30% to 35%, 35% to 40%, 40% to 45% or 45% to 50%), at a dose 1.5 nM.

In some embodiments of the isolated oligonucleotide of the present disclosure wherein the sense strand comprises a nucleotide that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region and that attenuates expression of the PCSK9 mRNA by 20% to 50%, at a dose of 1.5 nM, the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′);
iv) an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′);
v) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
vi) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′);
vii) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′); or
viii) an antisense strand of nucleic acid sequence according to SEQ ID NO: 29 (5′ UUAAUAAAAAUGCUACAAAACC 3′), and a sense strand of nucleic acid sequence according to SEQ ID NO: 31 (5′ UUUUGUAGCAUUUUUAUUAA 3′).

In some embodiments, the isolated oligonucleotide of the present disclosure can comprises a linker, sometimes referred to as a loop. siRNAs comprising a linker or loop are sometimes referred to as short hairpin RNAs (shRNAs). In some embodiments, both the sense and the antisense regions of the siRNA are encoded by one single-stranded RNA. In these embodiments, and the antisense region and the sense region hybridize to form a duplex region. The sense and antisense regions are joined by a linker sequence, forming a “hairpin” or “stem-loop” structure. The siRNA can have complementary sense and antisense regions at opposing ends of a single stranded molecule, so that the molecule can form a duplex region with the complementary sequence portions, and the strands are linked at one end of the duplex region by a linker. The linker can be either a nucleotide or non-nucleotide linker or a combination thereof. The linker can interact with the first, and optionally, second strands through covalent bonds or non-covalent interactions.

Any suitable nucleotide linker sequence is envisaged as within the scope of the disclosure. An siRNA of this disclosure may include a nucleotide, non-nucleotide, or mixed nucleotide/non-nucleotide linker that joins the sense region of the nucleic acid to the antisense region of the nucleic acid. A nucleotide linker can be a linker of >2 nucleotides in length, for example about 3, 4, 5, 6, 7, 8, 9, 10, 11, 12, 13, 14, 15 or 16 nucleotides in length.

Examples of a non-nucleotide linker include an abasic nucleotide, polyether, polyamine, polyamide, peptide, carbohydrate, lipid, polyhydrocarbon, or other polymeric agents, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc, 1991, Vol. 113, pp. 6324-6326; Jaeschke et al., Tetrahedron Lett., 1993, Vol. 34, pp. 301; Arnold et al., WO 1989/002439; Usman et al., WO 1995/006731; Dudycz et al., WO 1995/011910, and Ferentz et al., J. Am. Chem. Soc, 1991, Vol. 113, pp. 4000-4002.

Examples of nucleotide linker sequences include, but are not limited to, AUG, CCC, UUCG, CCACC, AAGCAA, CCACACC and UUCAAGAGA.

In some embodiments, the isolated oligonucleotide of the present disclosure is an siRNA that can be a dsRNA of a length suitable as a Dicer substrate, which can be processed to produce a RISC active siRNA molecule. See, e.g., Rossi et al., US2005/0244858.

A Dicer substrate double stranded RNA (dsRNA) can be of a length sufficient that it is processed by Dicer to produce an active siRNA, and may further include one or more of the following properties: (i) the Dicer substrate dsRNA can be asymmetric, for example, having a 3′ overhang on the antisense strand, (ii) the Dicer substrate dsRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can from 19-30 bp in length.

In some embodiments, the isolated oligonucleotide of the present disclosure comprises at least one modified nucleotide. In some embodiments of the isolated oligonucleotide of the present disclosure, the sense strand or the antisense strand or both comprise one or more modified nucleotide(s). In some embodiments, only the sense strand comprises one or more modified nucleotide(s). In some embodiments, only the antisense strand comprises one or more modified nucleotide(s). In some embodiments, both the sense strand and antisense strand comprise one or more modified nucleotide(s). In some embodiments, the isolated oligonucleotide is partially chemically modified. In some embodiments, the isolated oligonucleotide is fully chemically modified.

In some embodiments, the isolated oligonucleotide comprises at least two modified nucleotides. In some embodiments, the isolated oligonucleotide comprises at least three modified nucleotides. In some embodiments, the isolated oligonucleotide comprises at least four modified nucleotides. In some embodiments, the isolated oligonucleotide comprises at least five modified nucleotides. In some embodiments, the isolated oligonucleotide comprises at least six modified nucleotides. In some embodiments, the isolated oligonucleotide comprises at least seven modified nucleotides. In some embodiments, the isolated oligonucleotide comprises at least eight modified nucleotides. In some embodiments, the isolated oligonucleotide comprises at least nine modified nucleotides. In some embodiments, the isolated oligonucleotide comprises at least ten modified nucleotides. In some embodiments, the isolated oligonucleotide comprises at least eleven modified nucleotides. In some embodiments, the isolated oligonucleotide comprises at least twelve modified nucleotides. In some embodiments, the isolated oligonucleotide comprises at least thirteen modified nucleotides. In some embodiments, the isolated oligonucleotide comprises at least fourteen modified nucleotides. In some embodiments, the isolated oligonucleotide comprises at least fifteen modified nucleotides. In some embodiments, the isolated oligonucleotide comprises at least sixteen modified nucleotides. In some embodiments, the isolated oligonucleotide comprises at least seventeen modified nucleotides. In some embodiments, the isolated oligonucleotide comprises at least eighteen modified nucleotides. In some embodiments, the isolated oligonucleotide comprises at least nineteen modified nucleotides. In some embodiments, the isolated oligonucleotide comprises at least twenty modified nucleotides. In some embodiments, the isolated oligonucleotide comprises more than twenty modified nucleotides. In some embodiments, the isolated oligonucleotide comprises between twenty and thirty modified nucleotides. In some embodiments, the isolated oligonucleotide comprises between thirty and forty modified nucleotides. In some embodiments, the isolated oligonucleotide comprises between forty and fifty modified nucleotides.

In some embodiments, the sense strand and/or the antisense strand of the isolated oligonucleotide each comprise at least one modified nucleotides. In some embodiments, the sense strand and/or the antisense strand of the isolated oligonucleotide each comprise at least two modified nucleotides. In some embodiments, the sense strand and/or the antisense strand of the isolated oligonucleotide each comprise at least three modified nucleotides. In some embodiments, the sense strand and/or the antisense strand of the isolated oligonucleotide each comprise at least four modified nucleotides. In some embodiments, the sense strand and/or the antisense strand of the isolated oligonucleotide each comprise at least five modified nucleotides. In some embodiments, the sense strand and/or the antisense strand of the isolated oligonucleotide each comprise at least six modified nucleotides. In some embodiments, the sense strand and/or the antisense strand of the isolated oligonucleotide each comprise at least seven modified nucleotides. In some embodiments, the sense strand and/or the antisense strand of the isolated oligonucleotide each comprise at least eight modified nucleotides. In some embodiments, the sense strand and/or the antisense strand of the isolated oligonucleotide each comprise at least nine modified nucleotides. In some embodiments, the sense strand and/or the antisense strand of the isolated oligonucleotide each comprise at least ten modified nucleotides. In some embodiments, the sense strand and/or the antisense strand of the isolated oligonucleotide each comprise at least eleven modified nucleotides. In some embodiments, the sense strand and/or the antisense strand of the isolated oligonucleotide each comprise at least twelve modified nucleotides. In some embodiments, the sense strand and/or the antisense strand of the isolated oligonucleotide each comprise at least thirteen modified nucleotides. In some embodiments, the sense strand and/or the antisense strand of the isolated oligonucleotide each comprise at least fourteen modified nucleotides. In some embodiments, the sense strand and/or the antisense strand of the isolated oligonucleotide each comprise at least fifteen modified nucleotides. In some embodiments, the sense strand and/or the antisense strand of the isolated oligonucleotide each comprise at least sixteen modified nucleotides. In some embodiments, the sense strand and/or the antisense strand of the isolated oligonucleotide each comprise at least seventeen modified nucleotides. In some embodiments, the sense strand and/or the antisense strand of the isolated oligonucleotide each comprise at least eighteen modified nucleotides. In some embodiments, the sense strand and/or the antisense strand of the isolated oligonucleotide each comprise at least nineteen modified nucleotides. In some embodiments, the sense strand and/or the antisense strand of the isolated oligonucleotide each comprise at least twenty modified nucleotides.

In some embodiments, wherein the isolated oligonucleotide comprises more than one modified nucleotide, at least a first nucleotide comprises a first modification and at least a second nucleotide comprises a second modification. In some embodiments, the first modification and second modification are different. In some embodiments, the at least first nucleotide and the at least second nucleotide are located on different strands of the isolated oligonucleotide. In some embodiments, the at least first nucleotide and the at least second nucleotide are located on the same strand of the isolated oligonucleotide.

In some embodiments of the isolated oligonucleotide, wherein the isolated oligonucleotide comprises more than one modified nucleotide, at least a first modified nucleotide comprises a first modification, and at least a second modified nucleotide comprises a second modification, and at least a third nucleotide comprises a third modification. In some embodiments, the isolated oligonucleotide comprises a first, a second, a third and a fourth modifications. In some embodiments, the isolated oligonucleotide comprises more than four modifications. In some embodiments, all modifications are on the sense strand. In some embodiments, all modifications are on the antisense strand. Any combination of locations of the modifications between the sense strand and antisense strand is envisaged within the isolated oligonucleotides of the present disclosure.

In some embodiments, the modified nucleotides are consecutively located on the sense strand or the antisense strand or both. In some embodiments, some but not all of the modified nucleotides are consecutively located on the sense strand or the antisense strand or both. In some embodiments, the modified nucleotides on the sense strand or the antisense strand or both are not consecutively located.

Envisaged within the present disclosure is an isolated oligonucleotide, wherein any nucleotide on the sense strand or antisense strand can be modified. In some embodiments, any nucleotide on the antisense strand can be modified. In some embodiments, any nucleotide on the antisense strand can be modified.

In some embodiments, the isolated oligonucleotide of the present disclosure comprises at least one modified nucleotide(s). In some embodiments, the one or more modified nucleotide(s) increases the stability or potency or both of the isolated oligonucleotide. In some embodiments, the one or more modified nucleotide(s) increases the stability of the RNA duplex, and siRNA.

Modifications that increase RNA stability include, but are not limited to locked nucleic acids. As used herein, the term “locked nucleic acid” or “LNA” includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. LNAs having a 2′-4′ cyclic linkage, as described in the International Patent Application WO 99/14226, WO 00/56746, WO 00/56748, and WO 00/66604, the contents of which are incorporated herein by reference.

In some embodiments of the isolated oligonucleotide of the present disclosure, the sense strand or the antisense strand or both comprise at least one nucleotide having a modified phosphate backbone. In some embodiments, the sense strand of the isolated oligonucleotide comprises at least one nucleotide having a modified phosphate backbone. In some embodiments, the antisense strand of the isolated oligonucleotide comprises at least one nucleotide having a modified phosphate backbone. In some embodiments, wherein the isolated oligonucleotide of the present disclosure comprises a modified phosphate backbone, the modified phosphate backbone comprises a modified phosphodiester bond. In some embodiments, the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage. In some embodiments, the modified phosphodiester bond comprises phosphorothioate, phosphorodithioate, methylphosphonate, phosphoramidate diester, mesyl phosphoramidate, or phosphonoacetate.

In some embodiments, the isolated oligonucleotide of the present disclosure comprises one or more non-natural base-containing nucleotide, a locked nucleotide, or an abasic nucleotide. In some embodiments, the one or more modified nucleotide comprises a phosphorothioate derivative or an acridinine substituted nucleotide. In some embodiments, the isolated oligonucleotides of the present disclosure comprise a phosphate mimic at the 5′-terminus of antisense strand, including but not limited to vinylphosphonate or other phosphate analogues. In some embodiments, the 5′-phosphate mimic is ethylphosphonate, vinylphosphonate or an analog thereof.

In some embodiments, the modified nucleotide comprises 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl-aminomethyluracil, 5-methoxyaminomethyl-2-thiouracil, beta-D-mannosylqueosine, 5′-methoxycarboxymethyluracil, 5-methoxyuracil, 2-methylthio-N-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2, 6-diaminopurine

In some embodiments of the isolated oligonucleotide of the present disclosure, the sense strand or the antisense strand or both comprise a terminal or internal nucleotide linked to one or more targeting ligands. In some embodiments, the terminal or internal nucleotide is linked to the one or more targeting ligands directly. In some embodiments, the terminal or internal nucleotide is linked to the one or more targeting ligands indirectly by a linker. In some embodiments, the one or more targeting ligands linked directly or indirectly to the terminal or internal nucleotide can further comprise a PK modulator. In some embodiments, the PK modulator is a competitive modulator, a positive allosteric modulator, a negative allosteric modulator or a neutral allosteric modulator. In some embodiments, the targeting ligand is selected from one or more of a carbohydrate, a peptide, a lipid, an antibody or a fragment thereof, an aptamer, an albumin, a fibrinogen, and a folate.

### Modification of the Nucleotides

Provided herein is an isolated oligonucleotide, comprising: (a) a sense strand comprising X1 nucleotides, wherein at least one nucleotide is modified with a first modification, each of the remaining nucleotides is independently modified with a second modification, and X1 is an integer selected from 13-36, wherein the first modification and the second modification are different; and (b) an antisense strand comprising X2 nucleotides, wherein at least one nucleotide is modified with a third modification, each of the remaining nucleotides is independently modified with a fourth modification, and X2 is an integer selected from 18-31, wherein the third modification and the fourth modification are different.

In some embodiments, the X1 nucleotides of the sense strand of the isolated oligonucleotide of the present disclosure, is 18-21 and the X2 nucleotides of the antisense strand of the isolated oligonucleotide of the present disclosure is 20-23. In some embodiments, the X1 nucleotides of the sense strand of the isolated oligonucleotide of the present disclosure, is 20 or 21 and the X2 nucleotides of the antisense strand of the isolated oligonucleotide of the present disclosure is 22 or 23. In some embodiments, the X2 nucleotides of the antisense strand of the isolated oligonucleotide of the present disclosure equals the X1 nucleotides of the sense strand of the isolated oligonucleotide of the present disclosure plus 2. In some embodiments, the X1 nucleotides of the sense strand of the isolated oligonucleotide of the present disclosure is 21 and the X2 nucleotides of the antisense strand of the isolated oligonucleotide of the present disclosure is 23. In some embodiments, the X1 nucleotides of the sense strand of the isolated oligonucleotide of the present disclosure is 20 and the X2 nucleotides of the antisense strand of the isolated oligonucleotide of the present disclosure is 22.

In some embodiments of the isolated oligonucleotide of the present disclosure, the isolated oligonucleotide comprises: (a) a sense strand comprising 20 nucleotides, wherein at least one nucleotide is modified with a first modification, each of the remaining nucleotides is independently modified with a second modification, wherein the first modification and the second modification are the same or different; and (b) an antisense strand comprising 22 nucleotides, wherein at least one nucleotide is modified with a third modification, each of the remaining nucleotides is independently modified with a fourth modification, wherein the third modification and the fourth modification are the same or different.

In some embodiments, the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the first modification is 2′-F modification, 2′-CN modification, 2′-N3 modification, or 2′-deoxy modification, or a stereoisomer thereof. In some embodiments, the first modification is 2′-F modification, 2′-CN modification, or 2′-N3 modification, or a stereoisomer thereof. In some embodiments, the first modification is 2′-F modification or a stereoisomer thereof.

In some embodiments, the second modification is modification of the sugar moiety of one or more of the remaining nucleotides at the 2′-position selected from 2′-C1-C6 alkyl, 2′-OR modification wherein R is C1-C6 alkyl optionally substituted with C1-C6 alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the second modification is 2′-OR modification, or morpholino replacement, or a combination thereof. In some embodiments, the second modification is 2′-OR modification. In some embodiments, the second modification is 2′-O-methyl modification or 2′-methoxyethoxy modification. In some embodiments, the second modification is 2′-O-methyl modification. In some embodiments, the second modification is morpholino replacement.

In some embodiments, the first modification is 2′-F modification or a stereoisomer thereof, and the second modification is 2′-O-methyl modification or 2′-methoxyethoxy modification. In some embodiments, the first modification is 2′-F modification or a stereoisomer thereof, and the second modification is 2′-O-methyl modification.

In some embodiments, the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the third modification is 2′-F modification, 2′-CN modification, 2′-N3 modification, or 2′-deoxy modification, or a stereoisomer thereof. In some embodiments, the third modification is 2′-F modification, 2′-CN modification, or 2′-N₃ modification, or a stereoisomer thereof. In some embodiments, the third modification is 2′-F modification or a stereoisomer thereof.

In some embodiments, the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the fourth modification is 2′-OR modification, or morpholino replacement, or a combination thereof. In some embodiments, the fourth modification is 2′-OR modification. In some embodiments, the fourth modification is 2′-O-methyl modification or 2′-methoxyethoxy modification. In some embodiments, the fourth modification is 2′-O-methyl modification. In some embodiments, the fourth modification is morpholino replacement.

In some embodiments, the third modification is 2′-F modification or a stereoisomer thereof, and the fourth modification is 2′-O-methyl modification or 2′-methoxyethoxy modification. In some embodiments, the third modification is 2′-F modification or a stereoisomer thereof, and the fourth modification is 2′-O-methyl modification.

### Sense Strand

In some embodiments of the isolated oligonucleotide of the present disclosure comprising a sense and an antisense strand, in the sense strand of the isolated oligonucleotide of the present disclosure, at least three nucleotides are modified with the first modification. In some embodiments, in the sense strand of the isolated oligonucleotide of the present disclosure, at least two of the at least three nucleotides modified with the first modification are consecutively located. In some embodiments, in the sense strand of the isolated oligonucleotide of the present disclosure, at least three of the at least three nucleotides modified with the first modification are consecutively located.

In some embodiments, in the sense strand of the isolated oligonucleotide of the present disclosure, in the sense strand at least four nucleotides are modified with the first modification. In some embodiments, in the sense strand of the isolated oligonucleotide of the present disclosure, at least three of the at least four nucleotides modified with the first modification are consecutively located. In some embodiments, in the sense strand of the isolated oligonucleotide of the present disclosure, at least four of the at least four nucleotides modified with the first modification are consecutively located.

In some embodiments, in the sense strand of the isolated oligonucleotide of the present disclosure, in the sense strand at least five nucleotides are modified with the first modification. In some embodiments, in the sense strand of the isolated oligonucleotide of the present disclosure, at least three of the at least five nucleotides modified with the first modification are consecutively located. In some embodiments, in the sense strand of the isolated oligonucleotide of the present disclosure, at least four of the at least five nucleotides modified with the first modification are consecutively located.

In some embodiments, in the sense strand of the isolated oligonucleotide of the present disclosure, the at least three nucleotides, the at least four nucleotides, or the at least five nucleotides modified with the first modification are located from position 10 to position 15 from the nucleotide complementary to the first nucleotide at the 5′-terminus of the antisense strand.

In some embodiments, in the sense strand of the isolated oligonucleotide of the present disclosure, two of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, one of the at least three nucleotides modified with the first modification is located at position 11 from the nucleotide complementary to the first nucleotide at the 5′-terminus of the antisense strand.

In some embodiments, in the sense strand of the isolated oligonucleotide of the present disclosure, three of the at least three nucleotides modified with the first modification are located at positions 11, 12 and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions 12, 13 and 14 from the nucleotide complementary to the first nucleotide at the 5′-terminus of the antisense. In some embodiments, in the sense strand of the isolated oligonucleotide of the present disclosure, three of the at least three nucleotides modified with the first modification are located at positions 10, 11 and 12 from the nucleotide complementary to the first nucleotide at the 5′-terminus of the antisense strand.

In some embodiments, in the sense strand of the isolated oligonucleotide of the present disclosure, one of the at least four nucleotides modified with the first modification is located at position 10 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 11 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 12 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 13 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 14 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 15 from the nucleotide complementary to the first nucleotide at the 5′-terminus of the antisense strand.

In some embodiments, in the sense strand of the isolated oligonucleotide of the present disclosure, the at least four nucleotides modified with the first modification are located at positions 10, 11, 12 and 13 from the nucleotide complementary to the first nucleotide at the 5′-terminus of the antisense strand.

In some embodiments, in the sense strand of the isolated oligonucleotide of the present disclosure, the at least five nucleotides modified with the first modification are located at positions 10, 11, 12, 13 and 15 from the nucleotide complementary to the first nucleotide at the 5′-terminus of the antisense strand.

In some embodiments of the isolated oligonucleotide of the present disclosure, the sense strand comprises five nucleotides modified with the first modification, wherein the five nucleotides modified with the first modification are located at positions 10, 11, 12, 13 and 15 from the nucleotide complementary to the first nucleotide at the 5′-terminus of the antisense strand.

In some embodiments, in the sense strand of the isolated oligonucleotide of the present disclosure, not all of the at least three nucleotides, the at least four nucleotides, or the at least five nucleotides modified with the first modification are consecutively located. In some embodiments, in the sense strand of the isolated oligonucleotide of the present disclosure, the at least three nucleotides, the at least four nucleotides, or the at least five nucleotides are modified with 2′-F modification.

In some embodiments, the sense strand of the isolated oligonucleotide of the present disclosure comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M), 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to any one of: SEQ ID NOs: 8-13, 30 or 31.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 8.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 8, the antisense strand comprises a nucleotide sequence according to SEQ ID NO: 2.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 9.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 9, the antisense strand comprises a nucleotide sequence according to SEQ ID NO: 3.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“N”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 10.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“N”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 10, the antisense strand comprises a nucleotide sequence according to SEQ ID NO: 4.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 11.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“N”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 11, the antisense strand comprises a nucleotide sequence according to SEQ ID NO: 5.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“N”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 12.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 12, the antisense strand comprises a nucleotide sequence according to SEQ ID NO: 6.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 13.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 13, the antisense strand comprises a nucleotide sequence according to SEQ ID NO: 7.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“N”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 30.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 30, the antisense strand comprises a nucleotide sequence according to SEQ ID NO: 28.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, the sense strand comprises a nucleotide sequence according to SEQ ID NO: 31.

In some embodiments of the isolated oligonucleotide of the present disclosure, wherein the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′, and the sense strand comprises a nucleotide sequence according to SEQ ID NO: 31, the antisense strand comprises a nucleotide sequence according to SEQ ID NO: 29.

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 4 (5′ UAAAAAUGCUACAAAACCCAGA 3′), and a sense strand of nucleic acid sequence according to SEQ ID NO: 10 (5′ UGGGUUUUGUAGCAUUUUUA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a nucleotide sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 5 (5′ UAAUAAAAAUGCUACAAAACCC 3′), and a sense strand of nucleic acid sequence according to SEQ ID NO: 11 (5′ GUUUUGUAGCAUUUUUAUUA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 28 (5′ UUUAAUAAAAAUGCUACAAAAC 3′), and a sense strand of nucleic acid sequence according to SEQ ID NO: 30 (5′ UUUGUAGCAUUUUUAUUAAA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

In some embodiments of the isolated oligonucleotide of the present disclosure, wherein the sense strand comprises a sequence that is identical to a region between the nucleotide positions 3543 to 3596, from the 5′ end of a PCSK9 mRNA sequence according to SEQ ID NO: 1, the double stranded region comprises an antisense strand of nucleic acid sequence according to SEQ ID NO: 29 (5′ UUAUUAAUAAAAAUGCUACAAA 3′), and a sense strand of nucleic acid sequence according to SEQ ID NO: 31 (5′ UGUAGCAUUUUUAUUAAUAA 3′), and the sense strand of the isolated oligonucleotide comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g is any one of 0-16, and wherein the sense strand is 5′(M)₀(F)₀(M)₅(F)₁(M)₁(F)₄(M)₉ 3′.

### Antisense Strand

In some embodiments, in the antisense strand of the isolated oligonucleotide of the present disclosure, at most seven nucleotides are modified with the third modification.

In some embodiments, in the antisense strand of the isolated oligonucleotide of the present disclosure, at most four of the at most seven nucleotides modified with the third modification are located from position 2 to position 8 from the first nucleotide at the 5′-terminus of the antisense strand. In some embodiments, in the antisense strand of the isolated oligonucleotide of the present disclosure, at least one of the at most seven nucleotides are modified with the third modification is located at position 2 from the first nucleotide at the 5′-terminus of the antisense strand.

In some embodiments, in the antisense strand of the isolated oligonucleotide of the present disclosure, at most two of the at most seven nucleotides modified with the third modification are consecutively located. In some embodiments, in the antisense strand of the isolated oligonucleotide of the present disclosure, the at most two consecutively located of the at most seven nucleotides modified with the third modification are located at positions 2 and 3 from the first nucleotide at the 5′-terminus of the antisense strand.

In some embodiments, in the antisense strand of the isolated oligonucleotide of the present disclosure, at least one of the at most seven nucleotides modified with the third modification is located at position 14 from the first nucleotide at the 5′-terminus of the antisense strand. In some embodiments, in the antisense strand of the isolated oligonucleotide of the present disclosure, two or three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, two of the at most seven nucleotides modified with the third modification are located at positions 2 and 5 from the first nucleotide at the 5′-terminus of the antisense strand. In some embodiments, in the antisense strand of the isolated oligonucleotide of the present disclosure, two of the at most seven nucleotides modified with the third modification are located at positions 2 and 3 from the first nucleotide at the 5′-terminus of the antisense strand.

In some embodiments, in the antisense strand of the isolated oligonucleotide of the present disclosure, three of the at most seven nucleotides modified with the third modification are located at positions 2, 3 and 5 from the first nucleotide at the 5′-terminus of the antisense strand.

In some embodiments, in the antisense strand of the isolated oligonucleotide of the present disclosure, one or two of the at most seven nucleotides modified with the third modification are located at positions selected from position 14 and 16 from the first nucleotide at the 5′-terminus of the antisense strand. In some embodiments, in the antisense strand of the isolated oligonucleotide of the present disclosure, two of the at most seven nucleotides modified with the third modification are located at positions 14 and 16 from the first nucleotide at the 5′-terminus of the antisense strand. In some embodiments, in the antisense strand of the isolated oligonucleotide of the present disclosure, the at most seven nucleotides are modified with 2′-F modification. In some embodiments, in the antisense strand of the isolated oligonucleotide of the present disclosure, one of the at most seven nucleotides modified with the third modification is located at position 14 from the first nucleotide at the 5′-terminus of the antisense strand. In some embodiments, in the antisense strand of the isolated oligonucleotide of the present disclosure, two of the at most seven nucleotides modified with the third modification is located at positions 14 and 16 from the first nucleotide at the 5′-terminus of the antisense strand.

In some embodiments of the isolated oligonucleotides of the present disclosure, wherein the antisense strand comprises at most seven nucleotides modified with the third modification, the at most seven nucleotides are modified with 2′-F modification. In some embodiments, in the antisense strand of the isolated oligonucleotide of the present disclosure, one of the at most seven nucleotides modified with the third modification is located at position 2 from the first nucleotide at the 5′-terminus of the antisense strand. In some embodiments, in the antisense strand of the isolated oligonucleotide of the present disclosure, one of the at most seven nucleotides modified with the third modification is located at position 3 from the first nucleotide at the 5′-terminus of the antisense strand. In some embodiments, in the antisense strand of the isolated oligonucleotide of the present disclosure, one of the at most seven nucleotides modified with the third modification is located at position 5 from the first nucleotide at the 5′-terminus of the antisense strand. In some embodiments, in the antisense strand of the isolated oligonucleotide of the present disclosure, one of the at most seven nucleotides modified with the third modification is located at position 7 from the first nucleotide at the 5′-terminus of the antisense strand. In some embodiments, in the antisense strand of the isolated oligonucleotide of the present disclosure, one of the at most seven nucleotides modified with the third modification is located at position 10 from the first nucleotide at the 5′-terminus of the antisense strand. In some embodiments, in the antisense strand of the isolated oligonucleotide of the present disclosure, one of the at most seven nucleotides modified with the third modification is located at position 14 from the first nucleotide at the 5′-terminus of the antisense strand. In some embodiments, in the antisense strand of the isolated oligonucleotide of the present disclosure, one of the at most seven nucleotides modified with the third modification is located at position 16 from the first nucleotide at the 5′-terminus of the antisense strand. In some embodiments, in the antisense strand of the isolated oligonucleotide of the present disclosure, the at most seven nucleotides modified with the third modification are located at positions 2, 3, 5, 7, 10, 14 and 16 from the first nucleotide at the 5′-terminus of the antisense strand.

In some embodiments, in the antisense strand of the isolated oligonucleotide of the present disclosure, the antisense strand comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to any one of SEQ ID NOs: 2-7, 28 or 29.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 2.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 2, the sense strand comprises a nucleotide sequence according to SEQ ID NO: 8.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 3.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₅(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 3, the sense strand comprises a nucleotide sequence according to SEQ ID NO: 9.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₅(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 4.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)(M)₁(F)₂(M)₁ 5′, and the antisense strand comprises a nucleotide sequence according to SEQ ID NO: 4, the sense strand comprises a nucleotide sequence according to SEQ ID NO: 10.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 5.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 5, the sense strand comprises a nucleotide sequence according to SEQ ID NO: 11.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 6.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 6, the sense strand comprises a nucleotide sequence according to SEQ ID NO: 12.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)_{c}(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 7.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 7, the sense strand comprises a nucleotide sequence according to SEQ ID NO: 13.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 28.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)(M)₁(F)₂(M)₁ 5′, and the antisense strand comprises a nucleotide sequence according to SEQ ID NO: 28, the sense strand comprises a nucleotide sequence according to SEQ ID NO: 30.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)₁(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, the antisense strand comprises a nucleotide sequence according to SEQ ID NO: 29.

In some embodiments of the isolated oligonucleotide of the present disclosure, wherein the antisense strand of the isolated oligonucleotide comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, l, m, n and o is any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′, and the antisense strand comprises a nucleotide sequence according to SEQ ID NO: 29, the sense strand comprises a nucleotide sequence according to SEQ ID NO: 31.

### Targeting Ligand

In some embodiments, in the sense strand or the antisense strand or both of the isolated oligonucleotide of the present disclosure, a terminal or internal nucleotide is linked to a targeting ligand. In some embodiments, the targeting ligand is attached to one or more nucleotides at the 5′ end of the sense strand of the isolated oligonucleotide of the present disclosure. In some embodiments, the targeting ligand is attached to one or more nucleotides at the 3′ end of the sense strand of the isolated oligonucleotide of the present disclosure. In some embodiments, the targeting ligand is attached to one or more nucleotides at the 5′ end of the antisense strand of the isolated oligonucleotide of the present disclosure. In some embodiments, the targeting ligand is attached to one or more nucleotides at the 3′ end of the antisense strand of the isolated oligonucleotide of the present disclosure. In some embodiments, the targeting ligand is attached to one or more nucleotides of the at least two single-stranded nucleotides at the 3′-terminus of the antisense strand of the isolated oligonucleotide of the present disclosure.

In some embodiments, the targeting ligand is selected from one or more of a carbohydrate, a peptide, a lipid, an antibody or a fragment thereof, an aptamer, an albumin, a fibrinogen, and a folate. In some embodiments, the targeting ligand binds to a surface protein on a cell expressing a target mRNA of the isolated oligonucleotide of the present disclosure. In some embodiments, the targeting ligand mediates entry of the isolated oligonucleotide of the present disclosure, into a cell expressing a target mRNA of the isolated oligonucleotide of the present disclosure.

In some embodiments, the targeting ligand is a therapeutic ligand. In some embodiments, the targeting ligand is a therapeutic antibody.

In some embodiments, the targeting ligand is attached to the isolated oligonucleotide of the present disclosure by a linker. In some embodiments, the linker is any one or a protein, a DNA, an RNA or a chemical compound. In some embodiments, the isolated oligonucleotide, the linker and the targeting ligand, of the present disclosure form a scaffold. As used herein, the term “scaffold” refers to a compound or complex that comprises a linker of the present disclosure, wherein the linker is covalently attached to either a ligand or an isolated oligonucleotide or both.

In some embodiments, the isolated oligonucleotide, the linker and the targeting ligand, of the present disclosure form a conjugate. As used herein, the term “conjugate” refers to a compound or complex that comprises an isolated oligonucleotide being covalently attached to a ligand via a linker of the present disclosure.

As used herein, the term “targeting ligand” or “ligand” refers to a moiety that, when being covalently attached to GalNAc an oligonucleotide), is capable of mediating its entry into, or facilitating or allowing its delivery to, a target site (e.g., a target cell or tissue). In some embodiments, the targeting ligand comprises a sugar ligand moiety (e.g., N-acetylgalactosamine (GalNAc)) which may direct uptake of an oligonucleotide into the liver.

In some embodiments, the targeting ligand binds to the asialoglycoprotein receptor (ASGPR). In some embodiments, the targeting ligand binds to (e.g., through ASGPR) the liver, such as the parenchymal cells of the liver.

Suitable targeting ligands include, but are not limited to, the ligands disclosed in Winkler (Ther. Deliv., 2013, 4(7): 791-809), PCT Patent Appl'n Pub. Nos. WO/2016/100401, WO/2012/089352, and WO/2009/082607, and U.S. Patent Appl'n Pub. Nos. 2009/0239814, 2012/0136042, 2013/0158824, and 2009/0247608, each of which is incorporated by reference.

In some embodiments, the targeting ligand comprises a carbohydrate moiety.

As used herein, “carbohydrate moiety” refers to a moiety which comprises one or more monosaccharide units each having at least six carbon atoms (which may be linear, branched or cyclic), with an oxygen, nitrogen or sulfur atom bonded to each carbon atom. In some embodiments, the carbohydrate moiety comprises a monosaccharide, a disaccharide, a trisaccharide, or a tetrasaccharide. In some embodiments, the carbohydrate moiety comprises an oligosaccharide containing from about 4-9 monosaccharide units. In some embodiments, the carbohydrate moiety comprises a polysaccharide (e.g., a starch, a glycogen, a cellulose, or a polysaccharide gum).

In some embodiments, the carbohydrate moiety comprises a monosaccharide, a disaccharide, a trisaccharide, or a tetrasaccharide. In some embodiments, the carbohydrate moiety comprises an oligosaccharide (e.g., containing from about four to about nine monosaccharide units). In some embodiments, the carbohydrate moiety comprises a polysaccharide (e.g., a starch, a glycogen, a cellulose, or a polysaccharide gum).

In some embodiments, the ligand is capable of binding to a human asialoglycoprotein receptor (ASGPR), e.g., human asialoglycoprotein receptor 2 (ASGPR2).

In some embodiments, the carbohydrate moiety comprises a sugar (e.g., one, two, or three sugar). In some embodiments, the carbohydrate moiety comprises galactose or a derivative thereof (e.g., one, two, or three galactose or the derivative thereof). In some embodiments, the carbohydrate moiety comprises N-acetylgalactosamine or a derivative thereof (e.g., one, two, or three N-acetylgalactosamine or the derivative thereof). In some embodiments, the carbohydrate moiety comprises N-acetyl-D-galactosylamine or a derivative thereof (e.g., one, two, or three N-acetyl-D-galactosylamine or the derivative thereof).

In some embodiments, the carbohydrate moiety comprises N-acetylgalactosamine (e.g., one, two, or three N-acetylgalactosamine). In some embodiments, the carbohydrate moiety comprises N-acetyl-D-galactosylamine (e.g., one, two, or three N-acetyl-D-galactosylamine).

In some embodiments, the carbohydrate moiety comprises mannose or a derivative thereof (e.g., mannose-6-phosphate). In some embodiments, the carbohydrate moiety further comprises a linking moiety that connects the one or more sugar (e.g., N-acetyl-D-galactosylamine) with a linker.

In some embodiments the linker comprises thioether (e.g., thiosuccinimide, or the hydrolysis analogue thereof), disulfide, triazole, phosphorothioate, phosphodiester, ester, amide, or any combination thereof. In some embodiments, the linker is a triantennary linking moiety. Suitable targeting ligands include, but are not limited to, the ligands disclosed in PCT Appl'n Pub. Nos. WO/2015/006740, WO/2016/100401, WO/2017/214112, WO/2018/039364, and WO/2018/045317, each of which is incorporated herein by reference.

In some embodiments, the targeting ligand comprises a lipid or a lipid moiety (e.g., one, two, or three lipid moiety). In some embodiments the lipid moiety comprises (e.g., one, two, of three of) C8-C24 fatty acid, cholesterol, vitamin, sterol, phospholipid, or any combination thereof.

In some embodiments, the targeting ligand comprises a peptide or a peptide moiety (e.g., one, two, or three peptide moiety). In some embodiments, the peptide moiety comprises (e.g., one, two, or three of) integrin, insulin, glucagon-like peptide, or any combination thereof. In some embodiments, the targeting ligand comprises an antibody or an antibody moiety (e.g., transferrin). In some embodiments, the targeting ligand comprises one, two, or three antibody moieties (e.g., transferrin).

In some embodiments, the targeting ligand comprises an oligonucleotide (e.g., aptamer or CpG). In some embodiments, the targeting ligand comprises one, two, or three oligonucleotides (e.g., aptamer or CpG).

In some embodiments, the ligand comprises: one, two, or three sugar (e.g., N-acetyl-D-galactosylamine); one, two, or three lipid moieties; one, two, or three peptide moieties; one, two, or three antibody moieties; one, two, or three oligonucleotides; or any combination thereof.

In some embodiments, the linker is attached to the isolated oligonucleotide of the present disclosure, via a phosphate group, or an analog of a phosphate group, in the isolated oligonucleotide.

In some embodiments, the ligand comprises a sugar ligand moiety (e.g., N-acetylgalactosamine (GalNAc)) which may direct uptake of an oligonucleotide into the liver

In some embodiments, the ligand comprises GalNAc, or a derivative thereof. In some embodiments, the ligand comprises a GalNAc G1b structure shown below.

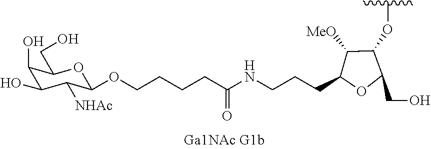 [SMILES: *O[C@H]1[C@@H](OC)[C@H](CCCNC(=O)CCCCOC2OC(CO)C(O)C(O)C2NC(C)=O)O[C@@H]1CO]

In some embodiments, the ligand comprises three GalNAc moieties, or three derivatives thereof. In some embodiments, the ligand comprises three GalNAc G1b moieties. In some embodiments, wherein the ligand comprises three GalNAc G1b moieties, the GalNAc G1b moieties are consecutively located. In some embodiments, the consecutively located GalNAc G1b moieties are located on the 3′ end of the sense strand. In some embodiments, wherein the ligand comprises three GalNAc G1b (“G1b”) moieties that are consecutively located, the first G1b moiety is linked to the second G1b moiety and the second G1b is linked to the third G1b moiety. In some embodiments, the first GalNAc G1b moiety is linked to the sense strand of the isolated oligonucleotide of the present disclosure.

In some embodiments of the isolated oligonucleotide of the present disclosure, wherein the ligand comprises three GalNAc G1b (“G1b”) moieties, wherein the first GalNAc G1b moiety is linked to the sense strand of the isolated oligonucleotide, the first GalNAc G1b moiety is also linked to the second GalNAc G1b moiety, and the second G1b is linked to the third G1b moiety. In some embodiments, wherein the ligand comprises three GalNAc G1b moieties, the three GalNAc G1b moieties are consecutively located on the 3′ end of the sense strand.

In some embodiments of the isolated oligonucleotide of the present disclosure, the isolated oligonucleotide is linked to the ligand (e.g., GalNAc G1b, or three GalNAc G1b moieties). In some embodiments, the isolated oligonucleotide is linked to the ligand via an internal or terminal nucleotide of the isolated oligonucleotide. In some embodiments, the isolated oligonucleotide is linked to the ligand via a ligand linker.

In some embodiments of the isolated oligonucleotide of the present disclosure, wherein the isolated oligonucleotide comprises a sense and an antisense strand, and wherein the ligand comprises three GalNAc G1b moieties, and the three GalNAc G1b moieties are consecutively located on the 3′ end of the sense strand, the ligand is linked to a terminal nucleotide on the sense strand of the isolated oligonucleotide. In some embodiments, the ligand is linked to a terminal nucleotide on the sense strand via a ligand linker. In some embodiments, the ligand linker is a monovalent linker. In some embodiments, the ligand linker is a bivalent linker. In some embodiments, the ligand linker is a trivalent linker.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, a targeting ligand is attached to the 3′ end of the sense strand. In some embodiments, the targeting ligand comprising three GalNAc G1b moieties.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, wherein the targeting ligand comprises three GalNAc G1b moieties attached to the 3′ end of the sense strand, the sense strand comprises a nucleic acid sequence according to SEQ ID NO: 8 (5′ CUUUUGUAACUUGAAGAUAA 3′); SEQ ID NO: 9 (5′ AACUUGAAGAUAUUUAUUCA 3′); SEQ ID NO: 10 (5′ UGGGUUUUGUAGCAUUUUUA 3′); SEQ ID NO: 11 (5′ GUUUUGUAGCAUUUUUAUUA 3′); SEQ ID NO: 12 (5′ UUUGUAGCAUUUUUAUUAAA 3′); SEQ ID NO: 13 (5′ UGUAGCAUUUUUAUUAAUAA 3′); SEQ ID NO: 30 (5′ AUUCUGGGUUUUGUAGCAUA 3′); or SEQ ID NO: 31 (5′ UUUUGUAGCAUUUUUAUUAA 3′).

The linkage at the 3′ end of the isolated oligonucleotide of the present disclosure may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 3′ terminal nucleotide. In some embodiments, the ligand described herein can be attached to the isolated oligonucleotide of the present disclosure with various ligand linkers that can be cleavable or non-cleavable.

In some embodiments of the isolated oligonucleotide of the present disclosure, wherein the ligand comprises three GalNAc G1b (“G1b”) moieties, wherein the first GalNAc G1b moiety is linked to the sense strand of the isolated oligonucleotide, the first GalNAc G1b moiety is also linked to the second GalNAc G1b moiety, and the second G1b is linked to the third G1b moiety. In some embodiments, wherein the ligand comprises three GalNAc G1b moieties, the three GalNAc G1b moieties are consecutively located on the 3′ end of the sense strand.

In some embodiments of the isolated oligonucleotide of the present disclosure, the isolated oligonucleotide is linked to the ligand (e.g., GalNAc G1b, or three GalNAc G1b moieties). In some embodiments, the isolated oligonucleotide is linked to the ligand via an internal or terminal nucleotide of the isolated oligonucleotide. In some embodiments, the isolated oligonucleotide is linked to the ligand via a ligand linker.

In some embodiments of the isolated oligonucleotide of the present disclosure, wherein the ligand comprises three GalNAc G1b (“G1b”) moieties, wherein the first GalNAc G1b moiety is linked to the sense strand of the isolated oligonucleotide, and the first GalNAc G1b moiety is also linked to the second GalNAc G1b moiety, the first GalNac G1b moiety is linked to the second GalNac G1b moiety through a linker comprising a phosphorothioate internucleotide linkage. In some embodiments, the second G1b is linked to the third G1b moiety. In some embodiments, wherein the second G1b is linked to the third G1b moiety through a linker comprising a phosphorothioate internucleotide linkage.

In some embodiments of the isolated oligonucleotide of the present disclosure, wherein the isolated oligonucleotide comprises a sense and an antisense strand, and wherein the ligand comprises three GalNAc G1b moieties, and the three GalNAc G1b moieties are consecutively located on the 3′ end of the sense strand, and the first GalNac G1b moiety is linked to the sense strand of the isolated oligonucleotide, the first GalNAc G1b moiety is also linked to the second GalNAc G1b moiety through a linker comprising a phosphorothioate internucleotide linkage, and the second G1b is linked to the third G1b moiety through a linker comprising a phosphorothioate internucleotide linkage.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, a targeting ligand is attached to the 3′ end of the sense strand. In some embodiments, the targeting ligand comprising three GalNAc G1b moieties, the three GalNAc G1b moieties are consecutively located on the 3′ end of the sense strand, wherein the first GalNac G1b moiety is linked to the sense strand of the isolated oligonucleotide, the first GalNAc G1b moiety is also linked to the second GalNAc G1b moiety through a linker comprising a phosphorothioate internucleotide linkage, and the second G1b is linked to the third G1b moiety through a linker comprising a phosphorothioate internucleotide linkage.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin/kexin type 9 (PCSK9) mRNA sequence according to SEQ ID NO: 1, and the antisense strand is substantially complementary to the sense strand such that the sense strand and the antisense strand together form a double stranded region, and the targeting ligand comprises three GalNAc G1b moieties attached to the 3′ end of the sense strand, wherein the first GalNac G1b moiety is linked to the sense strand of the isolated oligonucleotide, the first GalNAc G1b moiety is also linked to the second GalNAc G1b moiety through a linker comprising a phosphorothioate internucleotide linkage, and the second G1b is linked to the third G1b moiety through a linker comprising a phosphorothioate internucleotide linkage, the sense strand comprises a nucleic acid sequence according to SEQ ID NO: 8 (5′ CUUUUGUAACUUGAAGAUAA 3′); SEQ ID NO: 9 (5′ AACUUGAAGAUAUUUAUUCA 3′); SEQ ID NO: 10 (5′ UGGGUUUUGUAGCAUUUUUA 3′); SEQ ID NO: 11 (5′ GUUUUGUAGCAUUUUUAUUA 3′); SEQ ID NO: 12 (5′ UUUGUAGCAUUUUUAUUAAA 3′); SEQ ID NO: 13 (5′ UGUAGCAUUUUUAUUAAUAA 3′); SEQ ID NO: 30 (5′ AUUCUGGGUUUUGUAGCAUA 3′); or SEQ ID NO: 31 (5′ UUUUGUAGCAUUUUUAUUAA 3′).

### Modification of the Phosphate Groups

### Modified Terminal Phosphate Groups

The present disclosure further provides oligonucleotides and conjugates containing modified phosphate groups (also referred to as phosphate mimics or phosphate derivatives) for nucleic acid delivery. The present disclosure also relates to uses of oligonucleotides and conjugates containing modified phosphate groups, e.g., in delivering nucleic acid and/or treating or preventing diseases.

In some embodiments, the present disclosure provides phosphate mimics of 5′-terminal nucleotides. Without wishing to be bound by theory, it is understood that, when being incorporated into oligonucleotides (e.g., at the 5′-terminus of the antisense strand), the phosphate mimics could improve the Ago2 binding/loading and enhance the metabolic stability of the oligonucleotides, thus enhancing the potency and duration of the isolated oligonucleotides (e.g., dsRNA or siRNA).

In some embodiments of the isolated oligonucleotides of the present disclosure, the oligonucleotides comprise 5′-terminal nucleotide modifications. In some embodiments, the 5′-terminal modifications provide the functional effect of a phosphate group, but are more stable in the environmental conditions that the oligonucleotide will be exposed to when administered to a subject. In some embodiments, the isolated oligonucleotide comprises phosphate mimics that are more resistant to phosphatases and other enzymes while minimizing negative impact on the oligonucleotide's function (e.g., minimizing any reduction in gene target knockdown when used as an RNAi inhibitor molecule).

In some embodiments, the 5′-terminal modification is a chemical modification. In some embodiments, the chemical modification enhances stability against nucleases or other enzymes that degrade or interfere with the structure or activity of the isolated oligonucleotide.

In some embodiments, the sense or antisense strand of the isolated oligonucleotides of the present disclosure comprise a 5′-terminal phosphate group. In some embodiments, the 5′-terminal phosphate group comprises an unmodified phosphate having the formula: —O—P(═O)(OH)OH. In some embodiments, the 5′-terminal phosphate group comprises a modified phosphate. In some embodiments, the 5′-terminal phosphate group comprises a modified phosphate having the formula —CH₂—P(═X)(OR¹)OR², wherein X is O or S, R¹ is H or C₁-C₆ alkyl, and R² is H or C₁-C₆ alkyl. In some embodiments, the modified phosphate is referred to as a “phosphate mimic”.

The term, “halo” or “halogen”, as used herein, refers to fluoro, chloro, bromo and iodo.

The term, “aryl”, as used herein, includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

The term, “alkyl” or “C₁-C₆ alkyl”, as used herein, is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms. In some embodiments, the straight chain alkyl has one carbon atom. In some embodiments, the straight chain alkyl has two carbon atoms.

In some embodiments, the phosphate mimic is linked to the 5′-terminus of the isolated oligonucleotides (e.g., siRNAs) as shown in the following formula:

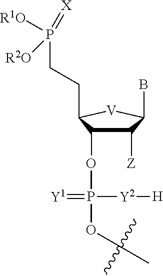

wherein:
B is H or a nucleobase moiety;
V is O or CH₂;
X is O or S;
R¹ is H or C₁-C₆ alkyl;
R² is H or C₁-C₆ alkyl;
Y¹ is O or S;
Y² is O or S;
Z is H, halogen, or —OR^{Z};
R^{Z} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Za};
each R^{Za} independently is halogen, C₁-C₆ alkyl, or —O—(C₁-C₆ alkyl), wherein the C₁-C₆ alkyl or —O—(C₁-C₆ alkyl) is optionally substituted with one or more halogen; and

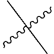

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

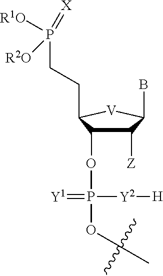

wherein:
B is H or a nucleobase moiety;
X is O or S;
R¹ is H or C₁-C₆ alkyl;
R² is H or C₁-C₆ alkyl;
Y¹ is O or S;
Y² is O or S;
Z is H, halogen, or —OR^{Z};
R^{Z} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Za};
each R^{Za} independently is halogen, C₁-C₆ alkyl, or —O—(C₁-C₆ alkyl), wherein the C₁-C₆ alkyl or —O—(C₁-C₆ alkyl) is optionally substituted with one or more halogen; and

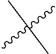

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

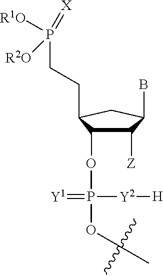

wherein:
B is H or a nucleobase moiety;
X is O or S;
R¹ is H or C₁-C₆ alkyl;
R² is H or C₁-C₆ alkyl;
Y¹ is O or S;
Y² is O or S;
Z is H, halogen, or —OR^{Z}.
R^{Z} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Za};
each R^{Za} independently is halogen, C₁-C₆ alkyl, or —O—(C₁-C₆ alkyl), wherein the C₁-C₆ alkyl or —O—(C₁-C₆ alkyl) is optionally substituted with one or more halogen; and

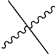

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

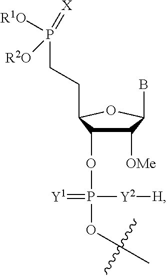

wherein:
B is H or a nucleobase moiety;
X is O or S;
R¹ is H or C₁-C₆ alkyl;
R² is H or C₁-C₆ alkyl;
Y¹ is O or S;
Y² is O or S; and

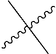

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

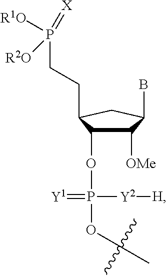

wherein:
B is H or a nucleobase moiety;
X is O or S;
R¹ is H or C₁-C₆ alkyl;
R² is H or C₁-C₆ alkyl;
Y¹ is O or S;
Y² is O or S; and

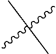

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula.

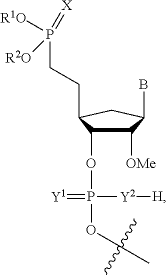

wherein:
B is H or a nucleobase moiety;
X is O or S;
R¹ is H or C₁-C₆ alkyl;
R² is H or C₁-C₆ alkyl; and

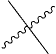

indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

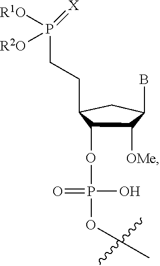

wherein:
B is H or a nucleobase moiety;
X is O or S;
R¹ is H or C₁-C₆ alkyl;
R² is H or C₁-C₆ alkyl; and

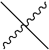

indicates an attachment to a nucleotide of the isolated oligonucleotide (e.g., siRNA).

In some embodiments, V is O.

In some embodiments, V is CH₂.

In some embodiments, X is O.

In some embodiments, X is S.

In some embodiments, R¹ is H.

In some embodiments, R¹ is C₁-C₆ alkyl (e.g., methyl, ethyl, n-propyl, i-propyl, n-butyl, i-butyl, s-butyl, t-butyl, pentyl, or hexyl).

In some embodiments, R¹ is methyl.

In some embodiments, R² is H.

In some embodiments, R² is C₁-C₆ alkyl (e.g., methyl, ethyl, n-propyl, i-propyl, n-butyl, i-butyl, s-butyl, t-butyl, pentyl, or hexyl).

In some embodiments, R² is methyl.

In some embodiments, Y¹ is O.

In some embodiments, Y¹ is S.

In some embodiments, Y² is O.

In some embodiments, Y² is S.

In some embodiments, Z is H.

In some embodiments, Z is not H.

In some embodiments, Z is halogen (e.g., F, Cl, Br, or I).

In some embodiments, Z is F or Cl.

In some embodiments, Z is F

In some embodiments, Z is —OR^{Z}.

In some embodiments, Z is —OH.

In some embodiments, Z is not —OH.

In some embodiments, Z is —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, Z is —OCH₃.

In some embodiments, Z is —O—(C₁-C₆ alkyl)-O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, Z is —OCH₂CH₂OCH₃.

In some embodiments, Z is —O—(C₁-C₆ alkyl)-(C₆-C₁₀ aryl) optionally substituted with one or more R^{Za}.

In some embodiments, Z is —O—(C₁-C₆ alkyl)-(C₆-C₁₀ aryl).

In some embodiments, Z is

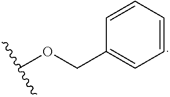 [SMILES: *OCc1ccccc1]

In some embodiments, Z is

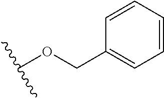 [SMILES: *OCc1ccccc1]

optionally substituted with one or more R^{Za}.

In some embodiments, Z is

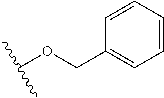 [SMILES: *OCc1ccccc1]

optionally substituted with one or more halogen.

In some embodiments, Z is

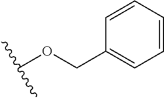 [SMILES: *OCc1ccccc1]

optionally substituted with one or more C₁-C₆ alkyl or —O—(C₁-C₆ alkyl), wherein the C₁-C₆ alkyl or —O—(C₁-C₆ alkyl) is optionally substituted with one or more halogen.

In some embodiments, R^{Z} is H.

In some embodiments, R^{Z} is not H.

In some embodiments, R^{Z} is C₁-C₆ alkyl (e.g., methyl, ethyl, n-propyl, i-propyl, n-butyl, i-butyl, s-butyl, t-butyl, pentyl, or hexyl) optionally substituted with one or more R^{Za}.

In some embodiments, R^{Z} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I) or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl) optionally substituted with one or more halogen.

In some embodiments, R^{Z} is C₁-C₆ alkyl (e.g., methyl, ethyl, n-propyl, i-propyl, n-butyl, i-butyl, s-butyl, t-butyl, pentyl, or hexyl).

In some embodiments, R^{Z} is methyl, ethyl, or propyl.

In some embodiments, R^{Z} is methyl.

In some embodiments, R^{Z} is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R^{Z} is C₁-C₆ alkyl (e.g., methyl, ethyl, n-propyl, i-propyl, n-butyl, i-butyl, s-butyl, t-butyl, pentyl, or hexyl) substituted with one or more —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the —O—(C₁-C₆ alkyl) is optionally substituted with one or more halogen.

In some embodiments, R^{Z} is —(C₁-C₆ alkyl)-(C₆-C₁₀ aryl) optionally substituted with one or more R^{Za}.

In some embodiments, R^{Z} is —(C₁-C₆ alkyl)-(C₆-C₁₀ aryl) optionally substituted with one or more halogen (e.g., F, Cl, Br, or I), C₁-C₆ alkyl (e.g., methyl, ethyl, n-propyl, i-propyl, n-butyl, i-butyl, s-butyl, t-butyl, pentyl, or hexyl), or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the C₁-C₆ alkyl or —O—(C₁-C₆ alkyl) is optionally substituted with one or more halogen.

In some embodiments, R⁷ is —(C₁-C₆ alkyl)-(C₆-C₁₀ aryl).

In some embodiments, at least one R^{Za} is halogen (e.g., F, Cl, Br, or I).

In some embodiments, at least one R^{Za} is F or Cl.

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl).

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, at least one R^{Za} is —O—(C₁-C₆ alkyl) optionally substituted with one or more halogen (e.g., F, Cl, Br, or I).

In some embodiments, at least one R^{Za} is —O—(C₁-C₆ alkyl).

In some embodiments, at least one R^{Za} is —O—(C₁-C₆ alkyl) substituted with one or more halogen (e.g., F, Cl, Br, or I).

In some embodiments, B is H.

In some embodiments, B is a nucleobase moiety.

The term “nucleobase moiety”, as used herein, refers to a nucleobase that is attached to the rest of the isolated oligonucleotides (e.g., dsRNA or siRNA) of the present disclosure, e.g., via an atom of the nucleobase or a functional group thereof.

In some embodiments, the nucleobase moiety is adenine (A), cytosine (C), guanine (G), thymine (T), or uracil (U).

In some embodiments, the nucleobase moiety is uracil (U).

In some embodiments, the phosphate mimic is linked to the 5′-terminus of the isolated oligonucleotides as shown in the following formula:

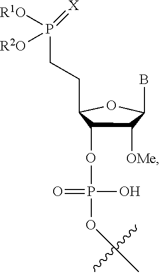

wherein:
B is a nucleobase moiety, wherein the nucleobase moiety is uracil (U), wherein the uracil is at position 1 from the 5′-terminus of the sense strand or at position 1 from the 5′-terminus of the antisense strand;
X is O;
R¹ is C₁ alkyl;
R² is H; and

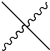

indicates an attachment to a nucleotide of the isolated oligonucleotide (e.g., siRNA).

In some embodiments, the phosphate mimic is linked to the 5′-terminus of the isolated oligonucleotides as shown in the following formula:

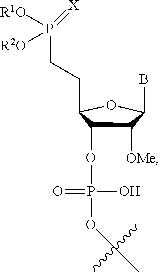

wherein:
B is a nucleobase moiety, wherein the nucleobase moiety is uracil (U), wherein the uracil is at position 1 from the 5′-terminus of the sense strand or at position 1 from the 5′-terminus of the antisense strand;
X is O;
R¹ is H;
R² is H; and

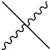

indicates an attachment to a nucleotide of the isolated oligonucleotide (e.g., siRNA).

In some embodiments, the phosphate mimic is linked to the 5′-terminus of the isolated oligonucleotides as shown in the following formula:

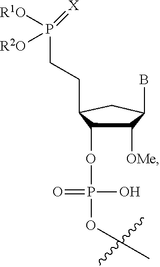

wherein:
B is a nucleobase moiety, wherein the nucleobase moiety is uracil (U), wherein the uracil is at position 1 from the 5′-terminus of the sense strand or at position 1 from the 5′-terminus of the antisense strand;
X is O;
R¹ is H;
R² is H; and

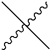

indicates an attachment to a nucleotide of the isolated oligonucleotide (e.g., siRNA).

In some embodiments of the isolated oligonucleotides of the present disclosure, the phosphate mimic is attached to the 5′-terminus of the antisense strand of the isolated oligonucleotide.

In some embodiments, the phosphate mimic is attached to a 5′-terminal uridine of the antisense strand of the isolated oligonucleotide, having the following structure (5′-MeEPmU):

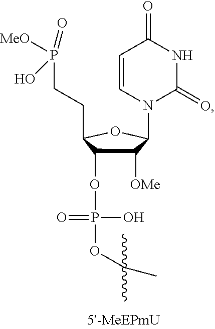

wherein “mU” is a 2′-O-methyl modified uridine nucleotide and “MeEP” is a mono methyl protected phosphate mimic.

In some embodiments, the phosphate mimic is attached to a 5′-terminal uridine of the antisense strand of the isolated oligonucleotide, having the following structure (5′-MeEPmUs):

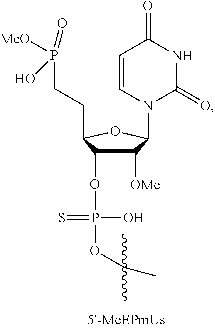

wherein “mU” is a 2′-O-methyl modified uridine nucleotide, “MeEP” is a mono methyl protected phosphate mimic, and “s” is a phosphorothioate internucleotide linkage.

The terms “5′-MeEP”, “5′-MeEP”, and “5′ MeEP” are used interchangeably herein.

In some embodiments, the phosphate mimic is attached to the 5′-terminal uridine of the antisense strand of the isolated oligonucleotide, having the following structure (5′-EPmU):

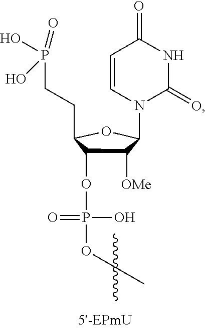

wherein “mU” is a 2′-O-methyl modified uridine nucleotide and “EP” is a phosphate mimic.

In some embodiments, the phosphate mimic is attached to the 5′ terminal uridine of the antisense strand of the isolated oligonucleotide, having the following structure (5′-C-EPmU):

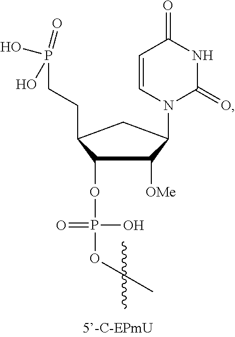

“wherein “mU” is a 2′-O-methyl modified uridine nucleotide and “C-EP” is a phosphate mimic.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin kexin 9 (PCSK9) mRNA sequence according to SEQ ID NO: 1, and the antisense strand is substantially complementary to the sense strand such that the sense strand and the antisense strand together form a double stranded region, the antisense strand comprises a mono methyl protected phosphate mimic (MeEP). In some embodiments, the MeEP is linked to the 5′ end of the antisense strand (5′-MeEP).

In some embodiments, wherein the MeEP is linked to the 5′ end of the antisense strand, the phosphate mimic is attached to a 5′-terminal uridine of the antisense strand.

In some embodiments, the 5′-terminal uridine is a 2′-O-methyl modified nucleotide.

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin kexin 9 (PCSK9) mRNA sequence according to SEQ ID NO: 1, and the antisense strand is substantially complementary to the sense strand such that the sense strand and the antisense strand together form a double stranded region, wherein the antisense strand comprises a 5′-MeEP linked to the 5′ end of the antisense strand, and wherein the antisense strand comprises a nucleic acid sequence according to SEQ ID NO: 2 (5′ UUAUCUUCAAGUUACAAAAGCAA 3′), SEQ ID NO: 3 (5′ UGAAUAAAUAUCUUCAAGUUAC 3′), SEQ ID NO: 4 (5′ UAAAAAUGCUACAAAACCCAGA 3′), SEQ ID NO: 5 (5′ UAAUAAAAAUGCUACAAAACCC 3′), SEQ ID NO. 6 (5′ UUUAAUAAAAAUGCUACAAAAC 3′), SEQ ID NO: 7 (5′ UUAUUAAUAAAAAUGCUACAAA 3′), SEQ ID NO: 28 (5′ UAUGCUACAAAACCCAGAAUAA 3′), or SEQ ID NO: 29 (5′ UUAAUAAAAAUGCUACAAAACC 3′).

In some embodiments of the isolated oligonucleotide of the present disclosure wherein the sense strand comprises a nucleotide sequence that is identical to a region between the nucleotide positions 3543 to 3596, from the 5′ end of a Proprotein convertase subtilisin kexin 9 (PCSK9) mRNA sequence according to SEQ ID NO: 1, and the antisense strand is substantially complementary to the sense strand such that the sense strand and the antisense strand together form a double stranded region, wherein the antisense strand comprises a 5′-MeEP linked to the 5′ end of the antisense strand, the sense strand comprises a targeting ligand comprising three GalNAc G1b moieties attached to the 3′ end of the sense strand.

### Modified Backbone Phosphate/Phosphodiester Bond

In some embodiments of the isolated oligonucleotide of the present disclosure, the sense strand or the antisense strand or both comprise at least one nucleotide having a modified phosphate backbone. In some embodiments, the sense strand of the isolated oligonucleotide comprises at least one nucleotide having a modified phosphate backbone. In some embodiments, the antisense strand of the isolated oligonucleotide comprises at least one nucleotide having a modified phosphate backbone. In some embodiments, wherein the isolated oligonucleotide of the present disclosure comprises a modified phosphate backbone, the modified phosphate backbone comprises a modified phosphodiester bond. A phosphodiester bond comprises a linkage having the formula:

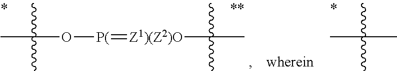

denotes attachment to a 3′ carbon of a first nucleotide in the isolated oligonucleotide of the present disclosure; and

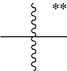

denotes attachment to a 5′ carbon of a second nucleotide in the isolated oligonucleotide of the present disclosure. In some embodiments, the phosphodiester bond is unmodified, wherein Z¹ is O and Z² is OH or O⁻. In some embodiments, the phosphodiester bond is modified, wherein Z¹ is O, S, NH, or N(C₁-C₆ alkyl) and Z² is OH, SH, NH₂, NH(C₁-C₆ alkyl), O⁻, S⁻, HN⁻, or (C₁-C₆ alkyl)N⁻, and wherein when Z¹ is O, Z² is not OH or O⁻.

In some embodiments, Z₁ is O.

In some embodiments, Z₁ is S.

In some embodiments, Z₁ is NH.

In some embodiments, Z₁ is N(C₁-C₆ alkyl).

In some embodiments, Z₂ is OH.

In some embodiments, Z₂ is SH.

In some embodiments, Z₂ is NH₂.

In some embodiments, Z₂ is NH(C₁-C₆ alkyl).

In some embodiments, Z₂ is SH, NH₂, or NH(C₁-C₆ alkyl).

In some embodiments, Z₂ is O⁻.

In some embodiments, Z₂ is S⁻.

In some embodiments, Z₂ is HN⁻.

In some embodiments, Z₂ is (C₁-C₆ alkyl)N⁻.

In some embodiments, Z₂ is S⁻, HN⁻, or (C₁-C₆ alkyl)N⁻.

In some embodiments, Zⱼ is 0 and Z₂ is SH.

In some embodiments, Z₁ is O and Z₂ is NH₂.

In some embodiments, Z₁ is O and Z₂ is NH(C₁-C₆ alkyl).

In some embodiments, Z₁ is S and Z₂ is OH.

In some embodiments, Z₁ is S and Z₂ is SH.

In some embodiments, Z₁ is S and Z₂ is NH₂.

In some embodiments, Z₁ is S and Z₂ is NH(C₁-C₆ alkyl).

In some embodiments, Z₁ is NH and Z₂ is OH.

In some embodiments, Z₁ is NH and Z₂ is SH.

In some embodiments, Zⱼ is NH and Z₂ is NH₂.

In some embodiments, Z₁ is NH and Z₂ is NH(C₁-C₆ alkyl).

In some embodiments, Z₁ is N(C₁-C₆ alkyl) and Z₂ is OH.

In some embodiments, Z₁ is N(C₁-C₆ alkyl) and Z₂ is SH.

In some embodiments, Z₁ is N(C₁-C₆ alkyl) and Z₂ is NH₂.

In some embodiments, Z₁ is N(C₁-C₆ alkyl) and Z₂ is NH(C₁-C₆ alkyl).

In some embodiments, Z₁ is O and Z₂ is S⁻.

In some embodiments, Z₁ is O and Z₂ is HN⁻.

In some embodiments, Z₁ is O and Z₂ is (C₁-C₆ alkyl)N⁻.

In some embodiments, Z₁ is S and Z₂ is O⁻.

In some embodiments, Z₁ is S and Z₂ is S⁻.

In some embodiments, Z₁ is S and Z₂ is HN⁻.

In some embodiments, Z₁ is S and Z₂ is (C₁-C₆ alkyl)N⁻.

In some embodiments, Z₁ is NH and Z₂ is O.

In some embodiments, Z₁ is NH and Z₂ is S⁻.

In some embodiments, Z₁ is NH and Z₂ is HN⁻.

In some embodiments, Z₁ is NH and Z₂ is (C₁-C₆ alkyl)N⁻.

In some embodiments, Z₁ is N(C₁-C₆ alkyl) and Z₂ is O⁻.

In some embodiments, Z₁ is N(C₁-C₆ alkyl) and Z₂ is S⁻.

In some embodiments, Z₁ is N(C₁-C₆ alkyl) and Z₂ is HN⁻.

In some embodiments, Z₁ is N(C₁-C₆ alkyl) and Z₂ is (C₁-C₆ alkyl)N⁻.

In some embodiments, the modified phosphodiester bond comprises a phosphorothioate internucleotide linkage.

In some embodiments, the modified phosphodiester bond comprises

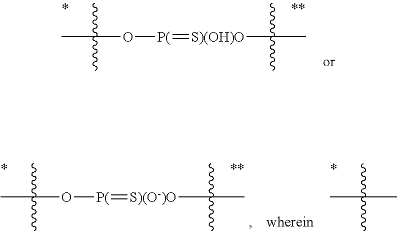

) denotes attachment to a 3′ carbon of a first nucleotide in the isolated oligonucleotide of the present disclosure; and

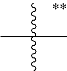

denotes attachment to a 5′ carbon of a second nucleotide in the isolated oligonucleotide of the present disclosure.

In some embodiments, the modified phosphodiester bond comprises

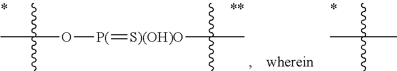

denotes attachment to a 3′ carbon of a first nucleotide in the isolated oligonucleotide of the present disclosure; and

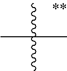

denotes attachment to a 5′ carbon of a second nucleotide in the isolated oligonucleotide of the present disclosure.

In some embodiments, the modified phosphodiester bond comprises

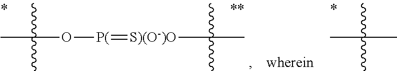

denotes attachment to a 3′ carbon of a first nucleotide in the isolated oligonucleotide of the present disclosure; and

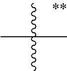

denotes attachment to a 5′ carbon of a second nucleotide in the isolated oligonucleotide of the present disclosure.

In some embodiments, the isolated oligonucleotide of the present disclosure comprises at least one modified phosphodiester bond(s). In some embodiments of the isolated oligonucleotide of the present disclosure, the sense strand or the antisense strand or both comprise one or more modified phosphodiester bonds. In some embodiments, only the sense strand comprises one or more modified phosphodiester bonds. In some embodiments, only the antisense strand comprises one or more modified phosphodiester bonds. In some embodiments, both the sense strand and antisense strand comprise one or more modified phosphodiester bonds.

In some embodiments, the isolated oligonucleotide comprises at least two modified phosphodiester bonds. In some embodiments, the isolated oligonucleotide comprises at least three modified phosphodiester bonds. In some embodiments, the isolated oligonucleotide comprises at least four modified phosphodiester bonds. In some embodiments, the isolated oligonucleotide comprises at least five modified phosphodiester bonds. In some embodiments, the isolated oligonucleotide comprises at least six modified phosphodiester bonds. In some embodiments, the isolated oligonucleotide comprises at least seven modified phosphodiester bonds. In some embodiments, the isolated oligonucleotide comprises at least eight modified phosphodiester bonds. In some embodiments, the isolated oligonucleotide comprises at least nine modified phosphodiester bonds. In some embodiments, the isolated oligonucleotide comprises at least ten modified phosphodiester bonds. In some embodiments, the isolated oligonucleotide comprises at least eleven modified phosphodiester bonds. In some embodiments, the isolated oligonucleotide comprises at least twelve modified phosphodiester bonds. In some embodiments, the isolated oligonucleotide comprises at least thirteen modified phosphodiester bonds. In some embodiments, the isolated oligonucleotide comprises at least fourteen modified phosphodiester bonds. In some embodiments, the isolated oligonucleotide comprises at least fifteen modified phosphodiester bonds. some embodiments, the isolated oligonucleotide comprises at least sixteen modified phosphodiester bonds. In some embodiments, the isolated oligonucleotide comprises at least seventeen modified phosphodiester bonds. In some embodiments, the isolated oligonucleotide comprises at least eighteen modified phosphodiester bonds. In some embodiments, the isolated oligonucleotide comprises at least nineteen modified phosphodiester bonds. In some embodiments, the isolated oligonucleotide comprises at least twenty modified phosphodiester bonds. In some embodiments, the isolated oligonucleotide comprises more than twenty modified phosphodiester bonds. In some embodiments, the isolated oligonucleotide comprises between twenty and thirty modified phosphodiester bonds. In some embodiments, the isolated oligonucleotide comprises between thirty and forty modified phosphodiester bonds. In some embodiments, the isolated oligonucleotide comprises between forty and fifty modified phosphodiester bonds.

In some embodiments, the isolated oligonucleotide comprises at least two phosphorothioate internucleotide linkages. In some embodiments, the isolated oligonucleotide comprises at least three phosphorothioate internucleotide linkages. In some embodiments, the isolated oligonucleotide comprises at least four phosphorothioate internucleotide linkages. In some embodiments, the isolated oligonucleotide comprises at least five phosphorothioate internucleotide linkages. In some embodiments, the isolated oligonucleotide comprises at least six phosphorothioate internucleotide linkages. In some embodiments, the isolated oligonucleotide comprises at least seven phosphorothioate internucleotide linkages. In some embodiments, the isolated oligonucleotide comprises at least eight phosphorothioate internucleotide linkages. In some embodiments, the isolated oligonucleotide comprises at least nine phosphorothioate internucleotide linkages. In some embodiments, the isolated oligonucleotide comprises at least ten phosphorothioate internucleotide linkages. In some embodiments, the isolated oligonucleotide comprises at least eleven phosphorothioate internucleotide linkages. In some embodiments, the isolated oligonucleotide comprises at least twelve phosphorothioate internucleotide linkages. In some embodiments, the isolated oligonucleotide comprises at least thirteen phosphorothioate internucleotide linkages. In some embodiments, the isolated oligonucleotide comprises at least fourteen phosphorothioate internucleotide linkages. In some embodiments, the isolated oligonucleotide comprises at least fifteen phosphorothioate internucleotide linkages. In some embodiments, the isolated oligonucleotide comprises at least sixteen phosphorothioate internucleotide linkages. In some embodiments, the isolated oligonucleotide comprises at least seventeen phosphorothioate internucleotide linkages. In some embodiments, the isolated oligonucleotide comprises at least eighteen phosphorothioate internucleotide linkages. In some embodiments, the isolated oligonucleotide comprises at least nineteen phosphorothioate internucleotide linkages. some embodiments, the isolated oligonucleotide comprises at least twenty phosphorothioate internucleotide linkages. In some embodiments, the isolated oligonucleotide comprises more than twenty phosphorothioate internucleotide linkages. In some embodiments, the isolated oligonucleotide comprises between twenty and thirty phosphorothioate internucleotide linkages. In some embodiments, the isolated oligonucleotide comprises between thirty and forty phosphorothioate internucleotide linkages. In some embodiments, the isolated oligonucleotide comprises between forty and fifty phosphorothioate internucleotide linkages.

In some embodiments, the sense strand and/or the antisense strand of the isolated oligonucleotide each comprise at least one modified phosphodiester bond(s). In some embodiments, the sense strand and/or the antisense strand of the isolated oligonucleotide each comprise at least two modified phosphodiester bonds. In some embodiments, the sense strand and/or the antisense strand of the isolated oligonucleotide each comprise at least three modified phosphodiester bonds. In some embodiments, the sense strand and/or the antisense strand of the isolated oligonucleotide each comprise at least four modified phosphodiester bonds. In some embodiments, the sense strand and/or the antisense strand of the isolated oligonucleotide each comprise at least five modified phosphodiester bonds. In some embodiments, the sense strand and/or the antisense strand of the isolated oligonucleotide each comprise at least six modified phosphodiester bonds. In some embodiments, the sense strand and/or the antisense strand of the isolated oligonucleotide each comprise at least seven modified phosphodiester bonds. In some embodiments, the sense strand and/or the antisense strand of the isolated oligonucleotide each comprise at least eight modified phosphodiester bonds. In some embodiments, the sense strand and/or the antisense strand of the isolated oligonucleotide each comprise at least nine modified phosphodiester bonds. In some embodiments, the sense strand and/or the antisense strand of the isolated oligonucleotide each comprise at least ten modified phosphodiester bonds. In some embodiments, the sense strand and/or the antisense strand of the isolated oligonucleotide each comprise at least eleven modified phosphodiester bonds. In some embodiments, the sense strand and/or the antisense strand of the isolated oligonucleotide each comprise at least twelve modified phosphodiester bonds. In some embodiments, the sense strand and/or the antisense strand of the isolated oligonucleotide each comprise at least thirteen modified phosphodiester bonds. In some embodiments, the sense strand and/or the antisense strand of the isolated oligonucleotide each comprise at least fourteen modified phosphodiester bonds. In some embodiments, the sense strand and/or the antisense strand of the isolated oligonucleotide each comprise at least fifteen modified phosphodiester bonds. In some embodiments, the sense strand and/or the antisense strand of the isolated oligonucleotide each comprise at least sixteen modified phosphodiester bonds. In some embodiments, the sense strand and/or the antisense strand of the isolated oligonucleotide each comprise at least seventeen modified phosphodiester bonds. In some embodiments, the sense strand and/or the antisense strand of the isolated oligonucleotide each comprise at least eighteen modified phosphodiester bonds. In some embodiments, the sense strand and/or the antisense strand of the isolated oligonucleotide each comprise at least nineteen modified phosphodiester bonds. In some embodiments, the sense strand and/or the antisense strand of the isolated oligonucleotide each comprise at least twenty modified phosphodiester bonds.

In some embodiments, the sense strand and/or the antisense strand of the isolated oligonucleotide each comprise at least one phosphorothioate internucleotide linkage(s). In some embodiments, the sense strand and/or the antisense strand of the isolated oligonucleotide each comprise at least two phosphorothioate internucleotide linkages. In some embodiments, the sense strand and/or the antisense strand of the isolated oligonucleotide each comprise at least three phosphorothioate internucleotide linkages. In some embodiments, the sense strand and/or the antisense strand of the isolated oligonucleotide each comprise at least four phosphorothioate internucleotide linkages. In some embodiments, the sense strand and/or the antisense strand of the isolated oligonucleotide each comprise at least five phosphorothioate internucleotide linkages. In some embodiments, the sense strand and/or the antisense strand of the isolated oligonucleotide each comprise at least six phosphorothioate internucleotide linkages. some embodiments, the sense strand and/or the antisense strand of the isolated oligonucleotide each comprise at least seven phosphorothioate internucleotide linkages. In some embodiments, the sense strand and/or the antisense strand of the isolated oligonucleotide each comprise at least eight phosphorothioate internucleotide linkages. In some embodiments, the sense strand and/or the antisense strand of the isolated oligonucleotide each comprise at least nine phosphorothioate internucleotide linkages. In some embodiments, the sense strand and/or the antisense strand of the isolated oligonucleotide each comprise at least ten phosphorothioate internucleotide linkages. In some embodiments, the sense strand and/or the antisense strand of the isolated oligonucleotide each comprise at least eleven phosphorothioate internucleotide linkages. In some embodiments, the sense strand and/or the antisense strand of the isolated oligonucleotide each comprise at least twelve phosphorothioate internucleotide linkages. In some embodiments, the sense strand and/or the antisense strand of the isolated oligonucleotide each comprise at least thirteen phosphorothioate internucleotide linkages. In some embodiments, the sense strand and/or the antisense strand of the isolated oligonucleotide each comprise at least fourteen phosphorothioate internucleotide linkages. In some embodiments, the sense strand and/or the antisense strand of the isolated oligonucleotide each comprise at least fifteen phosphorothioate internucleotide linkages. In some embodiments, the sense strand and/or the antisense strand of the isolated oligonucleotide each comprise at least sixteen phosphorothioate internucleotide linkages. In some embodiments, the sense strand and/or the antisense strand of the isolated oligonucleotide each comprise at least seventeen phosphorothioate internucleotide linkages. In some embodiments, the sense strand and/or the antisense strand of the isolated oligonucleotide each comprise at least eighteen phosphorothioate internucleotide linkages. In some embodiments, the sense strand and/or the antisense strand of the isolated oligonucleotide each comprise at least nineteen phosphorothioate internucleotide linkages. In some embodiments, the sense strand and/or the antisense strand of the isolated oligonucleotide each comprise at least twenty phosphorothioate internucleotide linkages.

In some embodiments, the modified phosphodiester bonds are consecutively located on the sense strand or the antisense strand or both. In some embodiments, some but not all of the modified phosphodiester bonds are consecutively located on the sense strand or the antisense strand or both. In some embodiments, the modified phosphodiester bonds on the sense strand or the antisense strand or both are not consecutively located.

Envisaged within the present disclosure is an isolated oligonucleotide, wherein any phosphodiester bond on the sense strand or antisense strand can be modified. In some embodiments, any phosphodiester bond on the antisense strand can be modified. In some embodiments, any phosphodiester bond on the antisense strand can be modified.

In some embodiments of the isolated oligonucleotide of the present disclosure, the antisense strand comprises between one and twenty, between one and fifteen, between one and ten, between one and five, or less than five modified phosphodiester bonds. In some embodiments, the between one and twenty, between one and fifteen, between one and ten, between one and five, or less than five modified phosphodiester bonds comprise phosphorothioate internucleotide linkages. In some embodiments, the antisense strand comprises less than five modified phosphodiester bonds. In some embodiments, the antisense strand comprises one, two, three, or four modified phosphodiester bonds. In some embodiments, wherein the antisense strand comprises one, two, three, or four modified phosphodiester bonds, the one, two, three, or four modified phosphodiester bonds comprise phosphorothioate internucleotide linkages. In some embodiments, the antisense strand comprises four modified phosphodiester bonds. In some embodiments, wherein the antisense strand comprises four modified phosphodiester bonds. the modified phosphodiester bonds comprise phosphorothioate.

In some embodiments, wherein the antisense strand comprises at least one, at least two, at least three, or at least four phosphorothioate internucleotide linkages, the phosphorothioate internucleotide linkages connect the nucleotides at position 1 and position 2 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 2 and position 3 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 20 and position 21 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 21 and position 22 from the first nucleotide at the 5′-terminus of the antisense strand. In some embodiments, wherein the antisense strand comprises at least one, at least two, at least three, or at least four modified phosphodiester bonds, wherein the modified phosphodiester bonds comprise phosphorothioate internucleotide linkages, the phosphorothioate internucleotide linkages are located between nucleotides at position 1 and 2, position 2 and 3, position 20 and 21, and position 21 and 22 from the first nucleotide at the 5′-terminus of the antisense strand.

In some embodiments of the isolated oligonucleotide of the present disclosure, wherein the antisense strand comprises at least one, at least two, at least three, or at least four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, wherein the antisense strand comprises at least four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, the antisense strand comprises four phosphorothioate internucleotide linkages. In some embodiments, wherein the antisense strand comprises four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, the sense strand comprises between one and twenty, between one and fifteen, between one and ten, between one and five, or less than five modified phosphodiester bonds. In some embodiments, the between one and twenty, between one and fifteen, between one and ten, between one and five, or less than five modified phosphodiester bonds comprise phosphorothioate internucleotide linkages. In some embodiments, the sense strand comprises less than five modified phosphodiester bonds. In some embodiments, wherein the sense strand comprises less than five modified phosphodiester bonds, the sense strand comprises one, two, three, or four modified phosphodiester bonds. In some embodiments, wherein the sense strand comprises one, two, three, or four modified phosphodiester bonds, the one, two, three, or four modified phosphodiester bonds comprise phosphorothioate internucleotide linkages. In some embodiments, the sense strand comprises four modified phosphodiester bonds. In some embodiments, wherein the sense strand comprises four modified phosphodiester bonds, the modified phosphodiester bonds comprise phosphorothioate internucleotide linkages.

In some embodiments, wherein the sense strand comprises at least one, at least two, at least three, or at least four modified phosphodiester bonds, the phosphodiester bonds comprise phosphorothioate internucleotide linkages. In some embodiments, wherein the sense strand comprises at least one, at least two, at least three, or at least four phosphorothioate internucleotide linkages, the phosphorothioate internucleotide linkages connect the nucleotides at position 1 and position 2 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 2 and position 3 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 18 and position 19 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 19 and position 20 from the first nucleotide at the 5′-terminus of the sense strand. In some embodiments, wherein the sense strand comprises at least one, at least two, at least three, or at least four modified phosphodiester bonds, wherein the modified phosphodiester bonds comprise phosphorothioate internucleotide linkages, the phosphorothioate internucleotide linkages are located between nucleotides at position 1 and 2, position 2 and 3, position 18 and 19, and position 19 and 20 from the first nucleotide at the 5′-terminus of the sense strand.

In some embodiments of the isolated oligonucleotide of the present disclosure, wherein the sense strand comprises at least one, at least two, at least three, or at least four phosphorothioate internucleotide linkages, the phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, wherein the sense strand comprises at least four phosphorothioate internucleotide linkages, the at least four phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, the sense strand comprises four phosphorothioate internucleotide linkages. In some embodiments, wherein the sense strand comprises four phosphorothioate internucleotide linkages, the phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, wherein the antisense strand and the sense strand comprise four phosphorothioate internucleotide linkages, the antisense strand comprises phosphorothioate internucleotide linkages located between nucleotides at position 1 to 3 and nucleotides at position 20 to 22 from the first nucleotide at the 5′-terminus of the antisense strand, and the sense strand comprises phosphorothioate internucleotide linkages located between nucleotides at position 1 to 3 and nucleotides at position 18 to 20 from the first nucleotide at the 5′-terminus of the sense strand.

In some embodiments of the isolated oligonucleotide of the present disclosure, the double stranded region comprises:
i) an antisense strand of nucleic acid sequence according to SEQ ID NO: 36 (5′ [mUs][fUs][fA][mU][fC][mU][fU][mC][mA][fA][mG][mU][mU][fA][mC][fA][mA][mA][mA][mGs][mCs][mA] 3′), and
a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) an antisense strand of nucleic acid sequence according to SEQ ID NO: 37 (5′ [mUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) i) an antisense strand of nucleic acid sequence according to SEQ ID NO: 38 (5′ [mUs][fAs][fA][mA][fA][mA][fU][mG][mC][fU][mA][mC][mA][fA][mA][fA][mC][mC][mC][mAs][mGs][mA] 3′), and
a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) an antisense strand of nucleic acid sequence according to SEQ ID NO: 39 (5′ [mUs][fAs][fA][mU][fA][mA][fA][mA][mA][fU][mG][mC][mU][fA][mC][fA][mA][mA][mA][mCs][mCs][mC] 3′), and
a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) an antisense strand of nucleic acid sequence according to SEQ ID NO: 40 (5′ [mUs][fUs][fU][mA][fA][mU][fA][mA][mA][fA][mA][mU][mG][fC][mU][fA][mC][mA][mA][mAs][mAs][mC] 3′), and
a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) an antisense strand of nucleic acid sequence according to SEQ ID NO: 41 (5′ [mUs][fUs][fA][mU][fU][mA][fA][mU][mA][fA][mA][mA][mA][fU][mG][fC][mU][mA][mC][mAs][mAs][mA] 3′), and
a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) an antisense strand of nucleic acid sequence according to SEQ ID NO: 42 (5′ [mUs][fAs][fU][mG][fC][mU][fA][mC][mA][fA][mA][mA][mC][fC][mC][fA][mG][mA][mA][mUs][mAs][mA] 3′), and
a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) an antisense strand of nucleic acid sequence according to SEQ ID NO: 43 (5′ [mUs][fUs][fA][mA][fU][mA][fA][mA][mA][fA][mU][mG][mC][fU][mA][fC][mA][mA][mA][mAs][mCs][mC] 3′), and
a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′); or
ix) an antisense strand of nucleic acid sequence according to SEQ ID NO: 37 (5′ [mUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′), wherein “m” is a 2′-O-methyl modified nucleotide, “f” is a 2′-F modified nucleotide, “s” is a phosphorothioate internucleotide linkage, and “G1b” is a GalNAc G1b moiety.

In some embodiments of the isolated oligonucleotide of the present disclosure, the double stranded region comprises:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′), and
a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) i) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′), and
a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′), and
a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′), and
a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′), and
a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′), and
a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) an antisense strand of nucleic acid sequence according to SEQ ID NO: 35 (5′ [MeEPmUs][fUs][fA][mA][fU][mA][fA][mA][mA][fA][mU][mG][mC][fU][mA][fC][mA][mA][mA][mAs][mCs][mC] 3′), and
a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′);
ix) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′);
x) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′); or
xi) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′),
wherein “m” is a 2′-O-methyl modified nucleotide, “f” is a 2′-F modified nucleotide, “s” is a phosphorothioate internucleotide linkage, “MeEP” is a mono methyl protected phosphate mimic, “EP” is a phosphate mimic, “C-EP” is a phosphate mimic, and “G1b” is a GalNAc G1b moiety.

In some embodiments of the isolated oligonucleotide of the present disclosure, the double stranded region comprises:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′), and
a sense strand of nucleic acid sequence according to SEQ ID NO: 44 (5′ [mCs][mUs][mU][mU][mU][fG][mU][fA][fA][fC][fU][mU][mG][mA][mA][mG][mA][mUs][mAs][mA][G1bs][G1bs][G1b] 3′);
ii) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 45 (5′ [mAs][mAs][mC][mU][mU][fG][mA][fA][fG][fA][fU][mA][mU][mU][mU][mA][mU][mUs][mCs][mA][G1bs][G1bs][G1b] 3′);
iii) i) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′), and
a sense strand of nucleic acid sequence according to SEQ ID NO: 46 (5′ [mUs][mGs][mG][mG][mU][fU][mU][fU][fG][fU][fA][mG][mC][mA][mU][mU][mU][mUs][mUs][mA][G1bs][G1bs][G1b] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′), and
a sense strand of nucleic acid sequence according to SEQ ID NO: 47 (5′ [mGs][mUs][mU][mU][mU][fG][mU][fA][fG][fC][fA][mU][mU][mU][mU][mU][mA][mUs][mUs][mA][G1bs][G1bs][G1b] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′), and
a sense strand of nucleic acid sequence according to SEQ ID NO: 48 (5′ [mUs][mUs][mU][mG][mU][fA][mG][fC][fA][fU][fU][mU][mU][mU][mA][mU][mU][mAs][mAs][mA][G1bs][G1bs][G1b] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′), and
a sense strand of nucleic acid sequence according to SEQ ID NO: 49 (5′ [mUs][mGs][mU][mA][mG][fC][mA][fU][fU][fU][fU][mU][mA][mU][mU][mA][mA][mUs][mAs][mA][G1bs][G1bs][G1b] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′), and
a sense strand of nucleic acid sequence according to SEQ ID NO: 50 (5′ [mAs][mUs][mU][mC][mU][fG][mG][fG][fU][fU][fU][mU][mG][mU][mA][mG][mC][mAs][mUs][mA][G1bs][G1bs][G1b] 3′); or
viii) an antisense strand of nucleic acid sequence according to SEQ ID NO: 35 (5′ [MeEPmUs][fUs][fA][mA][fU][mA][fA][mA][mA][fA][mU][mG][mC][fj][mA][fC][mA][mA][mA][mAs][mCs][mC] 3′), and
a sense strand of nucleic acid sequence according to SEQ ID NO: 51 (5′ [mUs][mUs][mU][mU][mG][fU][mA][fG][fC][fA][fU][mU][mU][mU][mU][mA][mU][mUs][mAs][mA][G1bs][G1bs][G1b] 3′);
ix) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 55 (5′ [mAs][mAs][mC][mU][mU][fG][mA][fA][fG][fA][fU][mA][mU][mU][mU][mA][mU][mU][mC][mA][G1bs][G1bs][G1b] 3′);
x) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 45 (5′ [mAs][mAs][mC][mU][mU][fG][mA][fA][fG][fA][fU][mA][mU][mU][mU][mA][mU][mUs][mCs][mA][G1bs][G1bs][G1b] 3′); or
xi) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 45 (5′ [mAs][mAs][mC][mU][mU][fG][mA][fA][fG][fA][fU][mA][mU][mU][mU][mA][mU][mUs][mCs][mA][G1bs][G1bs][G1b] 3′);
wherein “m” is a 2′-O-methyl modified nucleotide, “f” is a 2′-F modified nucleotide, “s” is a phosphorothioate internucleotide linkage, “MeEP” is a mono methyl protected phosphate mimic, “EP” is a phosphate mimic, “C-EP” is a phosphate mimic, and “G1b” is a GalNAc G1b moiety.

### Nucleic Acids and Vectors

The present disclosure also provides a vector encoding an isolated oligonucleotide disclosed herein. In some embodiments, the vector is any one of a plasmid, a cosmid or a viral vector. In some embodiments, the vector is an adenoviral vector. In some embodiments, the vector is a lentiviral vector. In some embodiments, the plasmid is an expression plasmid.

The disclosure provides nucleic acids comprising the sequences encoding the isolated oligonucleotides of the present disclosure (e.g., dsRNAs or siRNAs) targeting PCSK9 described herein.

In some embodiments, the nucleic acids are ribonucleic acids (RNAs). In some embodiments, the nucleic acids are deoxyribonucleic acids (DNAs). The DNAs may be a vector or a plasmid, e.g., an expression vector.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

By the term “express” or “expression” of a polynucleotide coding sequence, it is meant that the sequence is transcribed, and optionally, translated. Typically, according to the present disclosure, expression of a coding sequence of the disclosure will result in production of the polypeptide of the disclosure. The entire expressed polypeptide or fragment can also function in intact cells without purification.

In some embodiments, the vector is an expression vector for manufacturing siRNAs of the disclosure. Exemplary expression vectors may comprise a sequence encoding the sense and/or antisense strand of the isolated oligonucleotide of the present disclosure, under the control of a suitable promoter for transcription. Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

The isolated oligonucleotide of the present disclosure (e.g., dsRNAs and siRNAs) can be expressed endogenously from plasmid or viral expression vectors, or from minimal expression cassettes, for example, PCR generated fragments comprising one or more promoters and an appropriate template or templates for transcribing the siRNA. Examples of commercially available plasmid-based expression vectors for shRNA include members of the pSilencer series (Ambion, Austin. Tex.) and pCpG-siRNA (InvivoGen. San Diego, Calif.). Examples of kits for production of PCR-generated shRNA expression cassettes include Silencer Express (Ambion, Austin, Tex.) and siXpress (Mirus, Madison. Wis.).

Viral vectors for the in vivo expression of the isolated oligonucleotides (e.g., siRNAs and dsRNAs) in eukaryotic cells are also contemplated as within the scope of the instant disclosure. Viral vectors may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the siRNA from the vector and methods of delivering the viral vector, for example incorporated within a nanoparticle, are within the ordinary skill of one in the art.

It will be apparent to those skilled in the art that any suitable vector, optionally incorporated into a nanoparticle, can be used to deliver the isolated oligonucleotides of the present disclosure (e.g., dsRNAs or siRNAs) described herein to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

### Delivery Systems

The present disclosure also provides a delivery system comprising the isolated oligonucleotide disclosed herein or vector of the present disclosure encoding an isolated oligonucleotide disclosed herein. In some embodiments, the delivery system is any one of a liposome, a nanoparticle, a polymer based delivery system or a ligand-conjugate delivery system. In some embodiments, the ligand-conjugate delivery system comprises one or more of an antibody, a peptide, a sugar moiety or a combination thereof.

In some embodiments, the delivery system of the present disclosure comprise nanoparticles comprising the isolated oligonucleotides of the present disclosure (e.g., siRNA or dsRNAs) targeting a PCSK9 mRNA for degradation.

In some embodiments, the nanoparticle comprises a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a deblock copolymer, a polymeric micelle or a hyperbranched macromolecule. In some embodiments, the polymer-based nanoparticle comprises a multiblock copolymer a diblock copolymer. In some embodiments, the polymer-based nanoparticle is pH responsive. In some embodiments, the polymer-based nanoparticle further comprises a buffering component.

In some embodiments, the delivery system comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published Application 20090023673. In other embodiments, the one or more lipids are one or more cationic lipids. One skilled in the art will recognize which liposomes are appropriate for siRNA encapsulation.

In some embodiments, the liposome or the nanoparticle of the present disclosure comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a nanocrystal. Exemplary nanocrystals are crystalline particles with at least one dimension of less than 1000 nanometers, preferably of less than 100 nanometers.

In some embodiments, the nanoparticle comprises a polymer based nanoparticle. In some embodiments, the polymer comprises a multiblock copolymer, a diblock copolymer, a polymeric micelle or a hyperbranched macromolecule. In some embodiments, the particle comprises one or more cationic polymers. In some embodiments, the cationic polymer is chitosan, protamine, polylysine, polyhistidine, polyarginine or poly(ethylene)imine. In other embodiments, the one or more polymers contain the buffering component, degradable component, hydrophilic component, cleavable bond component or some combination thereof.

In some embodiments, the nanoparticles or some portion thereof are degradable. In other embodiments, the lipids and/or polymers of the nanoparticles are degradable.

In some embodiments, any of these delivery systems of the present disclosure can comprise a buffering component. In other embodiments, any of the of the present disclosure can comprise a buffering component and a degradable component. In still other embodiments, any of the of the present disclosure can comprise a buffering component and a hydrophilic component. In yet other embodiments, any of the of the present disclosure can comprise a buffering component and a cleavable bond component. In yet other embodiments, any of the of the present disclosure can comprise a buffering component, a degradable component and a hydrophilic component. In still other embodiments, any of the of the present disclosure can comprise a buffering component, a degradable component and a cleavable bond component. In further embodiments, any of the of the present disclosure can comprise a buffering component, a hydrophilic component and a cleavable bond component. In yet another embodiment, any of the of the present disclosure can comprise a buffering component, a degradable component, a hydrophilic component and a cleavable bond component. In some embodiments, the particle is composed of one or more polymers that contain any of the aforementioned combinations of components.

In some embodiments of the isolated oligonucleotides of the present disclosure, the delivery system comprises a ligand-conjugate delivery system. In some embodiments, the ligand-conjugate delivery system comprises one or more of an antibody, a peptide, a sugar moiety, lipid or a combination thereof

In further embodiments, the isolated oligonucleotide of the present disclosure targeting a PCSK9 mRNA (e.g., siRNA or dsRNA) is conjugated to, complexed to, or encapsulated by the one or more lipids or polymers of the delivery system. In further embodiments, the isolated oligonucleotide of the present disclosure targeting a PCSK9 mRNA (e.g., siRNA or dsRNA) can be encapsulated in the hollow core of a nanoparticle. Alternatively, or in addition, the isolated oligonucleotide of the present disclosure targeting a PCSK9 mRNA (e.g., siRNA or dsRNA) can be incorporated into the lipid or polymer based shell of the delivery system, for example via intercalation. Alternatively, or in addition, the isolated oligonucleotide of the present disclosure targeting a PCSK9 mRNA (e.g., siRNA or dsRNA) can be attached to the surface of the delivery system. In some embodiments, the isolated oligonucleotide of the present disclosure targeting a PCSK9 mRNA (e.g., siRNA or dsRNA) is conjugated to one or more lipids or polymers of the delivery system, e.g., via covalent attachment.

In some embodiments, the ligand conjugate delivery system further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

In some embodiments, the isolated oligonucleotide disclosed herein may further comprise a ligand that facilitates delivery or uptake of the isolated oligonucleotide to a particular tissue or cell, such as a liver cell. In certain embodiments, the ligand targets delivery of the RNAi construct to hepatocytes. In these and other embodiments, the ligand may comprise galactose, galactosamine or N-acetyl-galactosamine (GalNAc). In certain embodiments, the ligand comprises a multivalent galactose or multivalent GalNAc moiety, such as a trivalent or tetravalent galactose or GalNAc moiety. The ligand can be covalently attached to the 5′ or 3′ end of the sense strand of the RNAi construct, optionally via a linker.

In some embodiments, the targeting agent comprises a binding partner for a cell surface protein that is upregulated or overexpressed or normally expressed in a target cell encoding PCSK9 mRNA and expressing PCSK9 protein. In some embodiments, the binding partner can be a transmembrane peptidoglycan expressed on the surface of many types of such cells. Targeting of cell surface protein by the delivery system of the present disclosure thus provides superior delivery and specificity of the compositions of the disclosure to target cells. In some embodiments, the target cell can be any one of an intestinal cell, an arterial cell, a cell of the cardiovascular system, a hepatocyte, a pancreatic cell or a combination thereof.

In some embodiments, the delivery system of the present disclosure comprises a polymer based delivery system. In some embodiments, polymer based delivery system comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In other embodiments, the polyalkylene oxide is polyethylene oxide (PEO).

In some embodiments, the delivery system of the present disclosure is a polymer based nanoparticle. Polymer based nanoparticles comprise one or more polymers. In some embodiments, the one or more polymers comprise a polyester, poly(ortho ester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In some embodiments, the one or more polymers comprise poly(lactic-co-glycolic acid) (PLGA). In some embodiments, the one or more polymers comprise poly(lactic acid) (PLA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the polymer-based nanoparticle comprises poly(lactic-co-glycolic acid) PLGA polymers. In some embodiments, the PLGA nanoparticle further comprises a targeting agent, as described herein.

In some embodiments, the delivery system of the present disclosure is a nanoparticle of average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

### Therapeutic Agents

In some embodiments, the delivery system of the present disclosure are administered with one or more additional therapeutic agents. In some embodiments, the additional therapeutic agents can be a steroid, an anti-inflammatory agent, an antibody, a fusion protein, a small molecule or combination thereof.

In some embodiments, the additional therapeutic agent is incorporated into a delivery system of the present disclosure comprising at least one isolated oligonucleotide targeting PCSK9, disclosed herein. In some embodiments, the additional therapeutic agent is conjugated to, complexed to, or encapsulated by the one or more lipids or polymers of the delivery system. Additional therapeutic agents can be encapsulated in the hollow core of delivery system. Alternatively, or in addition, Additional therapeutic agents can be incorporated into the lipid or polymer based shell of the delivery system, for example via intercalation. Alternatively, or in addition, additional therapeutic agents can be attached to the surface of the delivery system. In some embodiments, the additional therapeutic agents are conjugated to one or more lipids or polymers of the delivery system, e.g. via covalent attachment.

In some embodiments, the additional therapeutic agent and the delivery system at least one isolated oligonucleotide targeting PCSK9, disclosed herein, are formulated in the same composition. For example, the delivery system comprising isolated oligonucleotide of the present disclosure targeting PCSK9 and the additional therapeutic agent can be formulated in the same pharmaceutical composition.

In some embodiments, the additional therapeutic agent and the delivery system comprises at least one isolated oligonucleotide targeting PCSK9, disclosed herein are formulated as separate compositions, e.g., for separate administration to a subject.

### Pharmaceutical Compositions

The present disclosure also provides a pharmaceutical composition comprising: an isolated oligonucleotide disclosed herein, a vector of the present disclosure encoding an isolated oligonucleotide disclosed herein, or a delivery system of the present disclosure, and a pharmaceutically acceptable carrier, diluent, or excipient.

The pharmaceutical compositions of the disclosure can optionally comprise therapeutic agents, pharmaceutical agents, carriers, adjuvants, dispersing agents, diluents, and the like.

In some embodiments, the pharmaceutical composition comprises a therapeutic agent, such as a chemotherapeutic agent. In some embodiments, the therapeutic agent is formulated in the delivery system comprising the one or more isolated oligonucleotides (e.g., dsRNA or siRNA) targeting PCSK9 of the present disclosure.

In some embodiments, an additional therapeutic agent is not formulated in the delivery system comprising the one or more isolated oligonucleotides (e.g., dsRNA or siRNA) targeting PCSK9 of the present disclosure, but both the delivery system and the therapeutic agent are formulated in the same pharmaceutical composition. In some embodiments, an additional therapeutic agent is not formulated in the delivery system comprising the one or more isolated oligonucleotides (e.g., dsRNA or siRNA) targeting PCSK9 of the present disclosure, and the delivery system and the therapeutic agent are formulated in separate pharmaceutical compositions.

Pharmaceutical compositions can contain any of the reagents discussed above, and one or more of a pharmaceutically acceptable carrier, a diluent or an excipient.

A pharmaceutical composition is in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed agent) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a of this disclosure include powders, sprays, ointments, pastes, creams, lotions, gels, solutions, patches and inhalants. In one embodiment, the active agent is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), intraperitoneal (into the body cavity) and transmucosal administration. Solutions or suspensions used for parenteral, intradermal, intraperitoneal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

The pharmaceutical compositions containing the nanoparticles described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active agents into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELT™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required nanoparticle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible pharmaceutically acceptable carrier. They can be enclosed in gelatin capsules or compressed into tablets. For the purpose of oral therapeutic administration, the active age can be incorporated with excipients and used in the form of tablets, troches, or capsules. Oral compositions can also be prepared using a fluid carrier for use as a mouthwash, wherein the agents in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the agents are delivered in the form of an aerosol spray from pressured container or dispenser, which contains a suitable propellant, e.g., a gas such as carbon dioxide, or a nebulizer.

The pharmaceutical compositions of the present disclosure can be prepared with pharmaceutically acceptable carriers that will protect the one or more isolated oligonucleotides (e.g., dsRNAs or siRNAs) targeting PCSK9 mRNA of the present disclosure against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, and the materials can be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms of the disclosure are dictated by and directly dependent on the unique characteristics of the active agent and the particular therapeutic effect to be achieved.

The pharmaceutical compositions can be included in a container, pack, or dispenser together with instructions for administration.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids.

Techniques for formulation and administration of the disclosed compositions of the disclosure can be found in Remington: the Science and Practice of Pharmacy, 19th edition, Mack Publishing Co., Easton, PA (1995).

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Methods of Making Isolated Oligonucleotides

Provided herein are methods of making the one or more oligonucleotides of (e.g., dsRNAs or siRNAs) targeting PCSK9 of the present disclosure, and delivery systems comprising same.

The one or more oligonucleotides of (e.g., dsRNAs or siRNAs) targeting PCSK9 of the present disclosure, may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers. The siRNAs can be purified by extraction with a solvent or resin, precipitation, electrophoresis, chromatography, or a combination thereof, for example. Alternatively, siRNAs may be used with little if any purification to avoid losses due to sample processing.

In some embodiments, the one or more oligonucleotides of (e.g., dsRNAs or siRNAs) targeting PCSK9 of the present disclosure can be produced using an expression vector into which a nucleic acid encoding the double stranded RNA has been cloned, for example under control of a suitable promoter.

In some embodiments, the one or more oligonucleotides of (e.g., dsRNAs or siRNAs) targeting PCSK9 of the present disclosure can be incorporated in a delivery system of the present disclosure (e.g., a nanoparticle).

Delivery systems comprising dsRNAs or siRNAs of the disclosure can be prepared by any suitable means known in the art. For example, polymeric nanoparticles can be prepared using various methods including, but not limited to, solvent evaporation, spontaneous emulsification, solvent diffusion, desolation, dialysis, ionic gelation, nanoprecipitation, salting out, spray drying and supercritical fluid methods. The dispersion of preformed polymers and the polymerization of monomers are two additional strategies for preparation of polymeric nanoparticles. However, the choice of an appropriate method depends upon various factors, which will be known to the person of ordinary skill in the art.

Sterile injectable solutions comprising a delivery system of the disclosure can be prepared by incorporating the one or more isolated oligonucleotides (e.g., dsRNA and siRNA) targeting PCSK9 disclosed herein, in the delivery systems (e.g. nanoparticle) in the required amount in an appropriate solvent with one or a combination of ingredients enumerated herein, as required, followed by filtered sterilization. Alternatively, or in addition, sterilization can be achieved through other means such as radiation or gas. Generally, dispersions are prepared by incorporating the delivery particles into a sterile vehicle that contains a basic dispersion medium and the required other ingredients from those enumerated above. In the case of sterile powders for the preparation of sterile injectable solutions, methods of preparation are vacuum drying and freeze drying that yields a powder of delivery system comprising the one or more isolated oligonucleotides (e.g., dsRNA and siRNA) targeting PCSK9 disclosed herein, plus any additional desired ingredient from a previously sterile filtered solution thereof.

### Methods of Use

The present disclosure also provides a method of inhibiting or downregulating the expression or level of PCSK9 in a subject in need thereof, wherein the method comprises administering to the subject an effective amount an isolated oligonucleotide disclosed herein, a vector of the of the present disclosure encoding an isolated oligonucleotide disclosed herein, a delivery system of the present disclosure, or a pharmaceutical composition of the present disclosure.

The present disclosure also provides a method of treating or preventing a disease or disorder associated with aberrant or increased expression or activity of PCSK9 or a disease or disorder where PCSK9 plays a role in a subject in need thereof, wherein the method comprises administering to the subject an effective amount an isolated oligonucleotide disclosed herein, a vector of the of the present disclosure encoding an isolated oligonucleotide disclosed herein, a delivery system of the present disclosure, or a pharmaceutical composition of the present disclosure.

The present disclosure also provides an isolated oligonucleotide disclosed herein, a vector of the of the present disclosure encoding an isolated oligonucleotide disclosed herein, a delivery system of the present disclosure, or a pharmaceutical composition of the present disclosure, for use in treatment or prevention of a disease or disorder associated with aberrant or increased expression or activity of PCSK9 or a disease or disorder where PCSK9 plays a role, in a subject in need thereof.

The present disclosure also provides use of an isolated oligonucleotide disclosed herein, a vector of the of the present disclosure encoding an isolated oligonucleotide disclosed herein, a delivery system of the present disclosure, or a pharmaceutical composition of the present disclosure, in the manufacture of a medicament for treatment or prevention of a disease or disorder associated with aberrant or increased expression or activity of PCSK9 or a disease or disorder where PCSK9 plays a role in a subject in need thereof.

Provided herein are methods of inhibiting or downregulating PCSK9 expression or activity in a cell, comprising contacting the cell with the one or more oligonucleotides (e.g., dsRNA or siRNA) targeting PCSK9 as described herein. The one or more oligonucleotides (e.g., dsRNA or siRNA) targeting PCSK9 as described herein can reduce or inhibit PCSK9 activity through the RNAi pathway. The cell can be in vitro, in vivo or ex vivo. For example, the cell can be from a cell line, or in vivo in a subject in need thereof.

In some embodiments, the one or more oligonucleotides (e.g., dsRNA or siRNA) targeting PCSK9 as described herein are capable of inducing RNAi-mediated degradation of an PCSK9 mRNA in a cell of a subject.

As used herein, the terms “contacting,” “introducing” and “administering” are used interchangeably, and refer to a process by which dsRNA or siRNA of the present disclosure or a nucleic acid molecule encoding a dsRNA or siRNA of this disclosure is delivered to a cell, in order to inhibit or alter or modify expression of a target gene. The dsRNA may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

In contrast, the term “increase” or grammatical variations thereof as used herein refers to an increase or elevation in the specified level or activity of at least about 5%, about 10%, about 15%, about 25%, about 35%, about 40%, about 50%, about 60%, about 75%, about 80%, about 90%, about 95% or more. Increases in activity can be described in terms of fold change. For example, activity can be increased 1.2×, 1.5×, 2×, 3×, 5×, 6×, 7×, 8×, 9×, 10× or more compared to a baseline level of activity.

As used herein, the term “IC50” or “IC₅₀ value” refers to the concentration of an agent where cell viability is reduced by half. The IC₅₀ is thus a measure of the effectiveness of an agent in inhibiting a biological process. In an exemplary model, cell lines are cultured using standard techniques, treated with any of the one or more oligonucleotides (e.g., dsRNA or siRNA) targeting PCSK9 as described herein, and the IC₅₀ value of the oligonucleotides (e.g., dsRNA or siRNA) targeting PCSK9 is calculated after 24, 48 and/or 72 hours to determine its effectiveness in downregulating or inhibiting the level of PCSK9 mRNA or protein to 50%, as compared to the level of PCSK9 mRNA or protein in an untreated cell or in the same cell before initiation of treatment with the isolated oligonucleotide.

Methods of monitoring of PCSK9 mRNA and/or protein expression can be used to characterize gene silencing, and to determine the effectiveness of the compositions described herein. Expression of PCSK9 may be evaluated by any known technique. Examples thereof include immunoprecipitations methods, utilizing PCSK9 antibodies in assays such as ELISAs, Western Blot, or immunohistochemistry to visualize PCSK9 protein expression in cells, or flow cytometry. Additional methods include various hybridization methods utilizing a nucleic acid that specifically hybridizes with a nucleic acid encoding PCSK9 or a unique fragment thereof, or a transcription product (e.g., mRNA) or splicing product of said nucleic acid, Northern Blot methods, Southern blot methods, and various PCR-based methods such as RT-PCR, qPCR or digital droplet PCR. PCSK9 mRNA expression may additionally be assessed using high throughput sequencing techniques.

Methods of assaying the effect of individual isolated oligonucleotides (e.g., dsRNA or siRNA) targeting PCSK9 include transfecting representative cell lines with isolated oligonucleotides, and measuring viability. For example, cells from representative cell lines can be transfected using methods known in the art, such as the Lipofectamine RNAiMAX (Invitrogen-13778-150, Carlsbad, CA), and cultured using any suitable technique known in the art. Optionally additional therapeutic agents as described herein can be added at variable concentrations to cell culture media following transfection. Following a suitable incubation period, such as 24-96 hours, cell viability can be measured using methods such as Cell Titer Glo 2.0 (Promega, CA) to determine cell viability, and/or PCSK9 mRNA and protein levels can be assessed using the methods described herein.

In some embodiments of the methods of inhibiting or downregulating PCSK9 expression or activity in a cell of the present disclosure, wherein the isolated oligonucleotide, the vector, the delivery system, or the pharmaceutical composition is administered parenterally.

In some embodiments of the methods of inhibiting or downregulating PCSK9 expression or activity in a cell of the present disclosure, wherein the parenteral administration is intravenous, subcutaneous, intraperitoneal, or intramuscular.

In some embodiments of the methods of inhibiting or downregulating PCSK9 expression or activity in a cell of the present disclosure, the subject is a human. In some embodiments of the methods of inhibiting or downregulating PCSK9 expression or activity in a cell of the present disclosure, the subject has nonalcoholic fatty liver disease (NAFLD) and related liver diseases like non-alcoholic steatohepatitis (NASH). In some embodiments of the methods of inhibiting or downregulating PCSK9 expression or activity in a cell of the present disclosure, the method comprises administering the isolated oligonucleotide, the vector, the delivery system, or the pharmaceutical composition, in combination with at least a second therapeutic agent.

In some embodiments of the methods of inhibiting or downregulating PCSK9 expression or activity in a cell of the present disclosure, the second therapeutic agent is an antibody, a small molecule drug, a peptide, a nucleotide molecule, or a combination thereof.

In some embodiments of the methods of inhibiting or downregulating PCSK9 expression or activity in a cell of the present disclosure, the second therapeutic agent is an isolated oligonucleotide of the present disclosure.

The present disclosure also provides a method of inhibiting or downregulating the expression or level of PCSK9 in a subject in need thereof, wherein the method comprises administering to the subject an effective amount of a first and at least a second oligonucleotides disclosed herein, wherein the first and at least second oligonucleotides comprise different sequences.

In some embodiments of the methods of inhibiting or downregulating PCSK9 expression or activity in a cell of the present disclosure, wherein the first and at least second oligonucleotides are administered simultaneously.

In some embodiments of the methods of inhibiting or downregulating PCSK9 expression or activity in a cell of the present disclosure, wherein the first and at least second oligonucleotides are administered sequentially.

In some embodiments of the methods of inhibiting or downregulating PCSK9 expression or activity in a cell of the present disclosure, the subject is a human. In some embodiments of the methods of inhibiting or downregulating PCSK9 expression or activity in a cell of the present disclosure, the subject has nonalcoholic fatty liver disease (NAFLD) and related liver diseases like non-alcoholic steatohepatitis (NASH). In some embodiments of the method of treating or preventing a disease or disorder associated with aberrant or increased expression or activity of PCSK9 or a disease or disorder where PCSK9 plays a role of the present disclosure, the subject is a human. In some embodiments of the method of treating or preventing a disease or disorder associated with aberrant or increased expression or activity of PCSK9 or a disease or disorder where PCSK9 plays a role of the present disclosure, the disease or disorder is nonalcoholic fatty liver disease (NAFLD) and related liver diseases like non-alcoholic steatohepatitis (NASH).

In some embodiments of the use for treating or preventing a disease or disorder associated with aberrant or increased expression or activity of PCSK9 or a disease or disorder where PCSK9 plays a role of the present disclosure, the subject is a human. In some embodiments of the use for treating or preventing a disease or disorder associated with aberrant or increased expression or activity of PCSK9 or a disease or disorder where PCSK9 plays a role of the present disclosure, the disease or disorder is nonalcoholic fatty liver disease (NAFLD) and related liver diseases like non-alcoholic steatohepatitis (NASH). In some embodiments of the use in the manufacture of a medicament for treatment or prevention of a disease or disorder associated with aberrant or increased expression or activity of PCSK9 of the present disclosure, the subject is a human. In some embodiments of the use in the manufacture of a medicament for treatment or prevention of a disease or disorder associated with aberrant or increased expression or activity of PCSK9 of the present disclosure of the present disclosure, the disease or disorder is nonalcoholic fatty liver disease (NAFLD) and related liver diseases like non-alcoholic steatohepatitis (NASH). The treatment or prevention of a disease or disorder is associated with aberrant or increased expression or activity of PCSK9.

### Routes of Administration

Nanoparticles comprising the one or more isolated oligonucleotides (e.g., dsRNA or siRNA) targeting PCSK9 mRNA of the present disclosure can be administered to a subject by many of the well-known methods currently used for therapeutic treatment. For example, for treatment of mammalian diseases associated with expression or activity of PCSK9, a compositions comprising the one or more isolated oligonucleotides (e.g., dsRNA or siRNA) targeting PCSK9 mRNA of the present disclosure may be injected directly into cells, injected into the blood stream or body cavities or taken orally or applied through the skin with patches. The dose chosen should be sufficient to constitute effective treatment but not so high as to cause unacceptable side effects. The state of the disease condition and the health of the patient should preferably be closely monitored during and for a reasonable period after treatment.

The compositions comprising the one or more isolated oligonucleotides (e.g., dsRNA or siRNA) targeting PCSK9 mRNA of the present disclosure can be administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the parenteral administration comprises intramuscular, intraperitoneal, subcutaneous or intravenous administration. One skilled in the art will recognize the advantages of certain routes of administration.

Compositions of the disclosure may be administered parenterally. Systemic administration of compositions comprising nanoparticles of the disclosure can also be by intravenous, transmucosal, subcutaneous, intraperitoneal, intramuscular or transdermal means. For intravenous parenteral administration, compositions comprising nanoparticles may be administered by injection or by infusion. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### Dosages

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing or treatment of the condition or symptom associated with expression or activity of PCSK9. Dosages may vary depending on the age and size of the subject and the type and severity of the disease or disorder associated with PCSK9 expression.

The term “effective amount” or “therapeutically effective amount”, as used interchangeably herein, refers to an amount of a pharmaceutical agent to treat, ameliorate, inhibit, downregulate or control the expression of PCSK9 or symptoms associated with aberrant or abnormal expression of PCSK9 in a subject, or to exhibit a detectable therapeutic or inhibitory effect in a subject. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any of the one or more isolated oligonucleotides (e.g., dsRNA or siRNA) targeting PCSK9 mRNA of the present disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. In some embodiments, a standard xenograft or patient derived xenograft mouse model can be used to determine the effectiveness of the one or more isolated oligonucleotides (e.g., dsRNA or siRNA) targeting PCSK9 mRNA of the present disclosure. Such information can then be used to determine useful doses and routes for administration in humans. Therapeutic/prophylactic efficacy and toxicity may be determined by standard pharmaceutical procedures in cell cultures or experimental animals, e.g., the maximum tolerated dose and no observable adverse effect dose. Pharmaceutical compositions that exhibit large therapeutic windows are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The dosage of nanoparticles comprising the one or more isolated oligonucleotides (e.g., dsRNA or siRNA) targeting PCSK9 mRNA of the present disclosure, required depends on the choice of the route of administration; the nature of the formulation; the nature of the patient's illness; the subject's size, weight, surface area, age, and sex; other drugs being administered; and the judgment of the attending physician. Wide variations in the needed dosage are to be expected in view of the differing efficiencies of various routes of administration. For example, oral administration would be expected to require higher dosages than administration by intravenous injection (e.g., 2-, 3-, 4-, 6-, 8-, 10-; 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., capsules or implantable devices) may increase the efficiency of delivery, particularly for oral delivery.

A therapeutically effective dose of the one or more isolated oligonucleotides (e.g., dsRNA or siRNA) targeting PCSK9 mRNA of the present disclosure, can optionally be combined with approved amounts of therapeutic agents, and described herein.

### Kits and Articles of Manufacture

The present disclosure also provides a kit comprising an isolated oligonucleotide disclosed herein, a vector of the present disclosure encoding an isolated oligonucleotide disclosed herein, a delivery system of the present disclosure, or a pharmaceutical composition of the present disclosure.

The kits are for use in the treatment of diseases related to abnormal or aberrant expression of PCSK9, in a mammal. The kits are for use in downregulating or inhibiting expression of PCSK9 partially or completely, in a mammal. In some embodiments, the mammal is a human, a mouse, a rat, a rabbit, a pig, a bovine, a canine, a feline, an ungulate, an ape, a monkey or an equine species. In some embodiments, the mammal is a human

Nanoparticles comprising the one or more isolated oligonucleotides (e.g., dsRNA or siRNA) targeting PCSK9 mRNA of the present disclosure, can be lyophilized before being packaged in the kit, or can be provided in solution with a pharmaceutically acceptable carrier, diluent of excipient.

In some embodiments of the kits of the disclosure, the kit comprises a therapeutically effective amount of a composition comprising the delivery system of the present disclosure comprising one or more of the isolated oligonucleotides of the present disclosure targeting PCSK9 (dsRNA or siRNA), and instructions for use of the same. In some embodiments, the kit further comprises at least one additional therapeutic agents, as described herein.

Articles of manufacture include, but are not limited to, instructions for use of the kit in treating diseases related to abnormal or aberrant expression of PCSK9 or diseases related to expression of PCSK9.

In some embodiments, the kits further comprise instructions for administering the isolated oligonucleotides, the vector, the delivery systems and the pharmaceutical compositions of the disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed invention. Based on the present disclosure the skilled artisan can identify and employ other components and methodology useful for practicing the present disclosure.

### EXAMPLES

### Example 1: Design and Testing of siRNA Compounds Against PCSK9 mRNA

The example described herein determined the potency of the siRNA compounds against PCSK9 mRNA, including compounds described in Tables 1-3.

### Materials and Methods

A set of siRNA compounds against human PCSK9 transcript (Accession No: NM_174936.4) were designed (Table 1). Due to the low expression levels of PCSK9 in the in vitro cell lines, a Dual-Glo Luciferase assay was performed to evaluate the compound potency in silencing human PCSK9 mRNA. Huh-7 cells were first transfected with psiCHECK2-PCSK9 plasmids with Fugene-HD reagents on day 0. On day 1, the siRNA compounds were diluted into the desired concentration with PBS and transfected into the psiCHECK2-PCSK9-plasmid transfected Huh-7 cells, at two concentrations of 0.05 nM and 0.5 nM, with Lipofectamine RNAiMAX reagents. After 24 hours post-siRNA transfection (day 2), Firefly (transfection control) and Renilla (fused to PCSK9 transcript sequence) luciferase activities were measured using the Dual-Glo Luciferase reagent kit. Data is represented as Mean+/−SEM.

### Compound Synthesis

Oligonucleotides were prepared by solid-phase synthesis according to standard protocols. Briefly, oligonucleotide synthesis was conducted on a solid support to incorporate each nucleoside phosphoramidites from 3′-end to 5′-end to prepare oligo single strands. ETT or BTT was used as an activator for the coupling reaction. Iodine in water/pyridine/THF was used to oxidize phosphite-triester (P(III)) to afford phosphate backbones and DDTT was used for the preparation of phosphorothioate linkages. Aqueous ammonium was used to cleave oligos from solid support and to remove protecting groups globally. The oligonucleotide crude was then concentrated by Genevac and purified by AEX-HPLC. The pure fractions were combined and concentrated, and their purity was analyzed by LC-MS. The oligonucleotides were then dialyzed against water using MidiTrap G-25 column, concentrated, and their OD amounts were measured.

To prepare siRNA duplexes, the sense and antisense strands were annealed at 95° C. for 10 min, based on equal molar amounts, and cooled down to room temperature. The duplex purity was determined by AEX-HPLC, and the solutions were lyophilized to afford the desired siRNA duplex powder.

### RT-qPCR

mRNA from cells were prepared using RNeasy 96 kit. Liver mRNA samples were prepared with RNeasy Plus mini kit. mRNAs were reverse transcribed into cDNAs using High-Capacity cDNA Reverse transcription kits with RNase Inhibitors. TaqMan multiplex qPCR assays were performed to determine the relative PCSK9 mRNA levels over time.

### In Vitro Compound Screening in Huh-7 Cells

Compounds (Table 1) were diluted into the desired concentration with PBS. The compounds were transfected into the cultured Huh-7 cells with Lipofectamine RNAiMAX reagents. At 24 hours post transfection, mRNA was extracted for RT-qPCR analysis.

### Western Blotting

10 mg of liver samples were homogenized in RIPA lysis buffer. Total protein concentrations were determined with BCA assays. 10 μg of total protein samples were loaded for electrophoresis. Cyno PCSK9 proteins were blotted with rabbit anti-PCSK9 (N-terminus) polyclonal antibody (Absin, Abs110383, 1:1000 dilution). GAPDH proteins were blotted with rabbit anti-GAPDH polyclonal antibody (Absin, Abs132004, 1:5000 dilution). Goat anti-rabbit IgG-HRP secondary antibodies (Absin, Abs20002, 1:20000) were used to detect the proteins.

### Ex Vivo Potency Evaluation in Primary Human Hepatocytes (PHH)

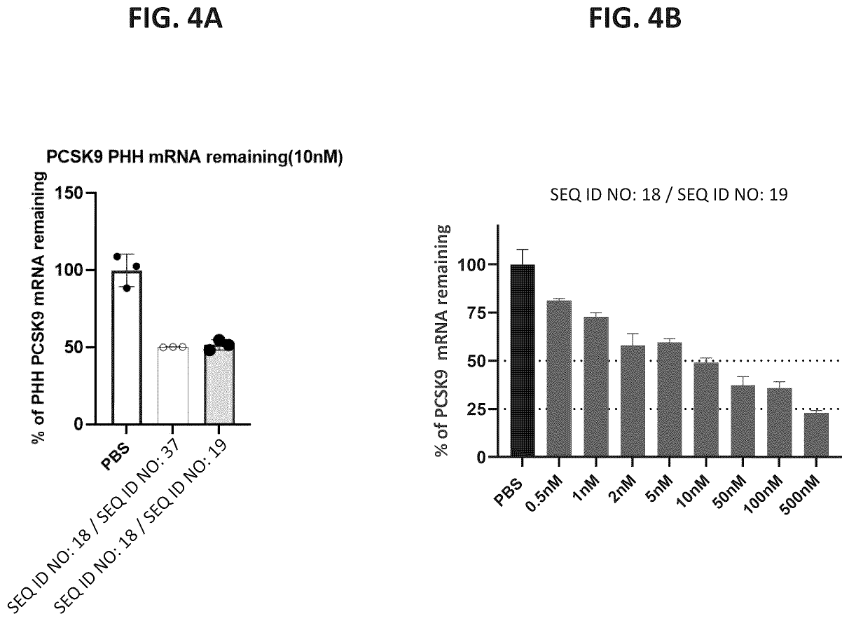

Compounds with GalNAc conjugations on the 3′ end of the sense (passenger) strand were tested through free-uptake in PHH. The compounds were directly added to the cultured primary hepatocytes at single or multiple doses. 48 hrs later, the cells were harvested for mRNA analysis through RT-qPCR. Data shown as Mean+/−SD (FIG. 4A-4B)

### In Vivo Compound Screening in HDI Mouse Liver

Compounds with GalNAc conjugations were formulated in 1×PBS dosed on day 1 through subcutaneous dosing to BALB/c female animals (6-8 weeks old). Animals then received 10 μg of pcDNA3.1-PCSK9 plasmids on day 4. Liver autopsies were taken on day 5 for RT-qPCR analysis.

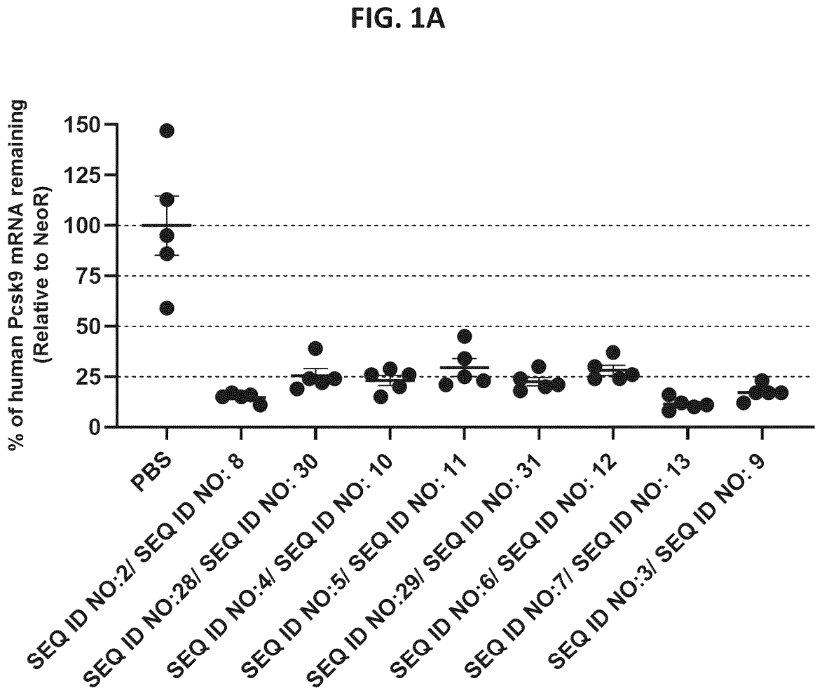

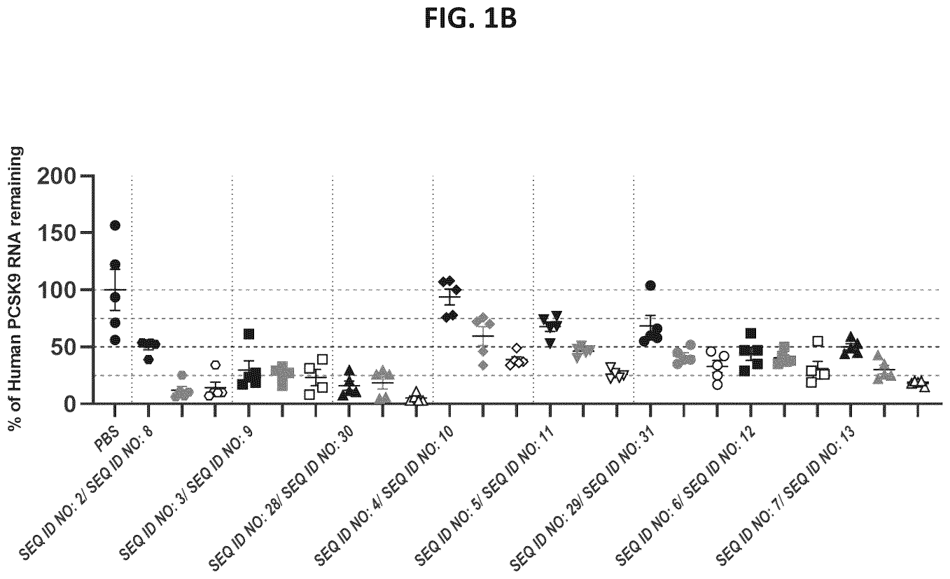

For dose response study, 6-8 weeks old female BALB/c mice were dosed subcutaneously at 0.25 mg/kg, 0.5 mg/kg, or 1 mg/kg. The control animals were dosed with PBS. Animals were sacrificed 4 days post-dose and liver samples were collected for RNA extraction and PCSK9 mRNA levels analysis by RT-qPCR. Results for this experiment are shown in FIGS. 1A-1B.

In Vivo Potency and Duration Evaluation in Macaca fascicularis

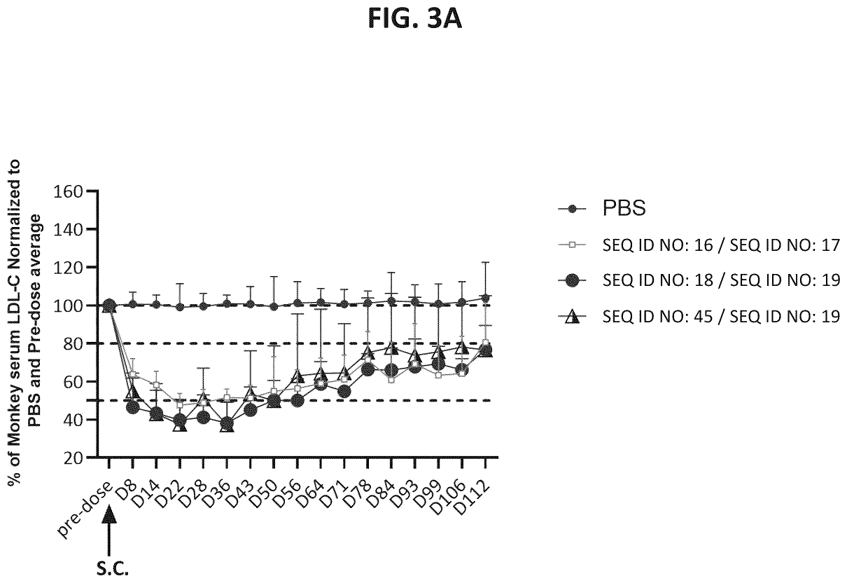

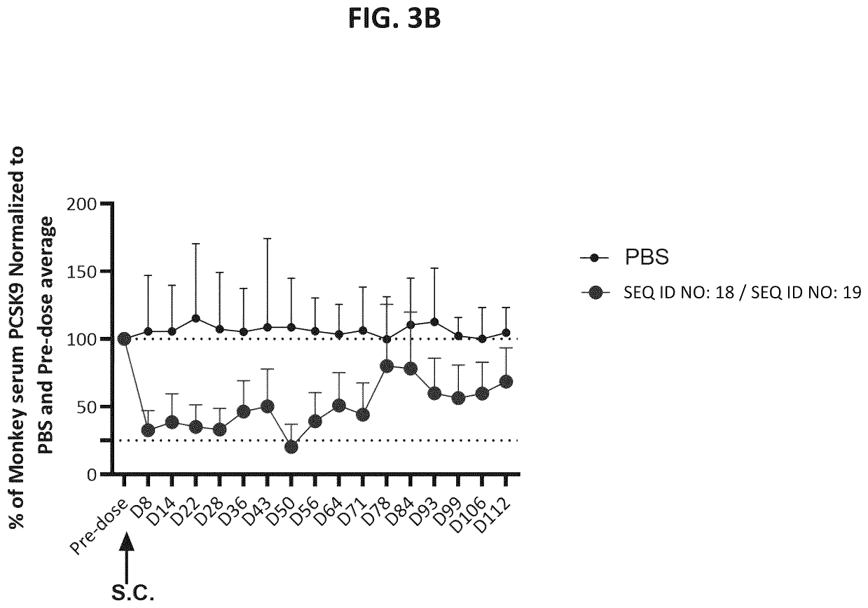

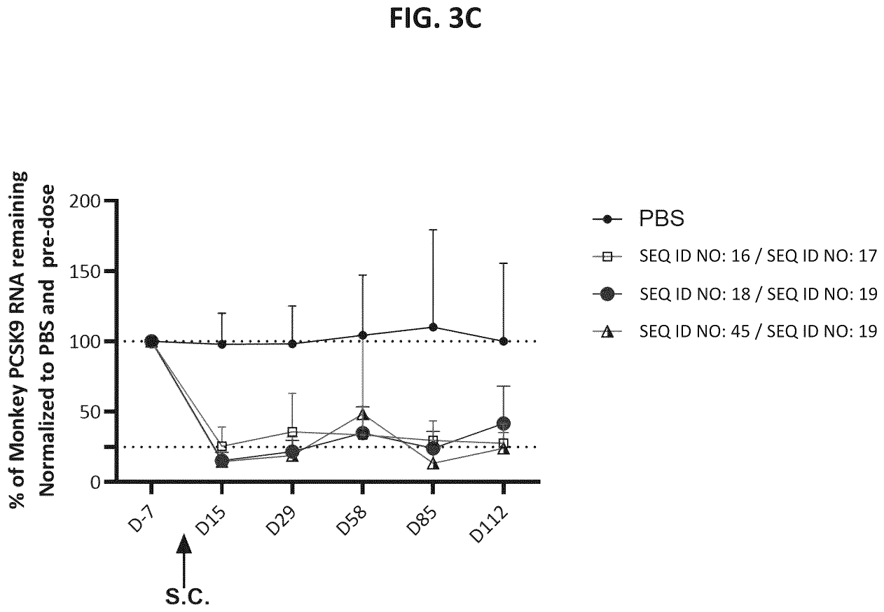

To evaluate compound potency (Table 3), nonterminal studies were conducted in cynomolgus macaques. A liver biopsy was taken from c) no monkeys in the study to determine the baseline mRNA expression level two weeks before dosing (Day −14). Pre-dosing serum LDL-c and PCSK9 protein levels were evaluated on day −14, day −7 and day 1 as baseline measurements. Animals were dosed with a single dose of 3 mg/kg of compounds listed in Table 3 on day 1. Blood was taken at day 8, 16, 22, 30, 36, 43, 50, 58, 64, 71, 78, 86, 93, 99, 106, and 113 post-dosing. Liver biopsies were collected on day 15, 29, and 85 post-dosing. Results for this experiment are shown in FIGS. 3A-3B.

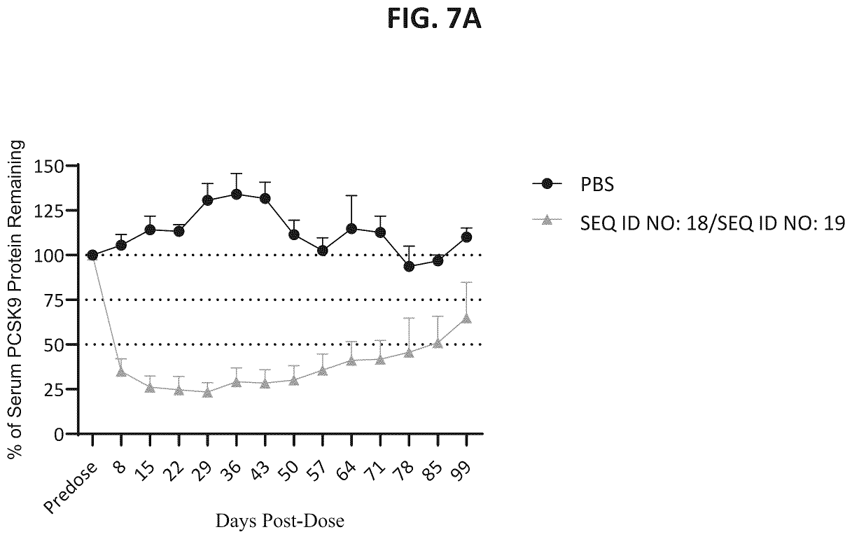

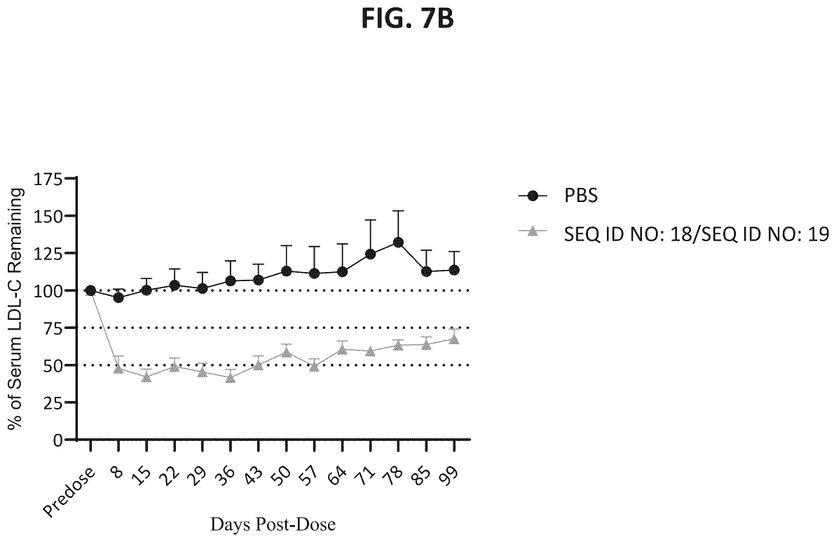

Additionally, monkeys with LDL-C levels greater than 1.5 mmol/L were used as a model for spontaneous hypercholesterolemia. These monkeys were dosed with compounds (Table 3) at 3 mg/kg subcutaneously on day 0. Serum samples were collected for PCSK9 and LDL-C analysis (FIGS. 7A-7B).

### In Vivo Potency and Duration Evaluation in Humanized PCSK9 Transgenic Mice

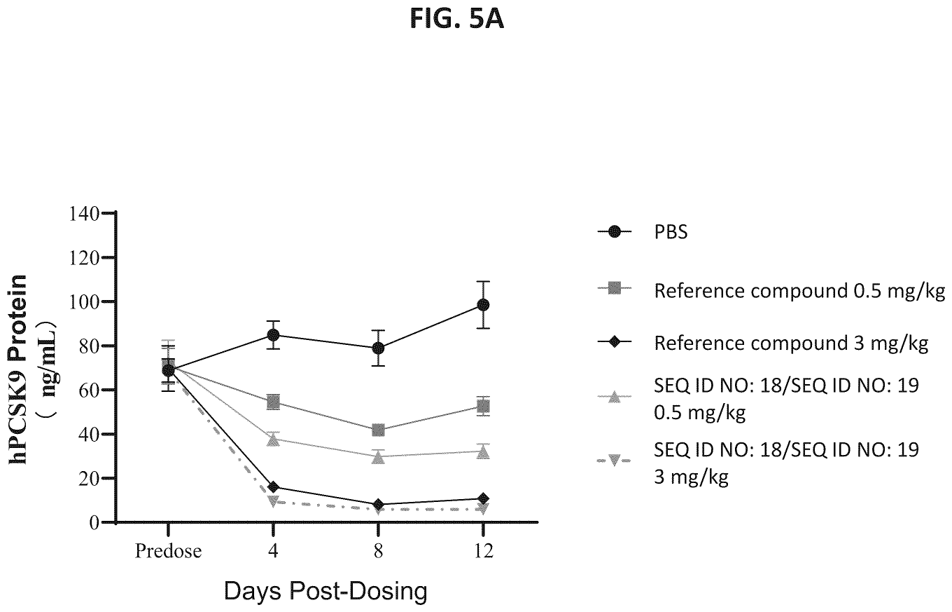

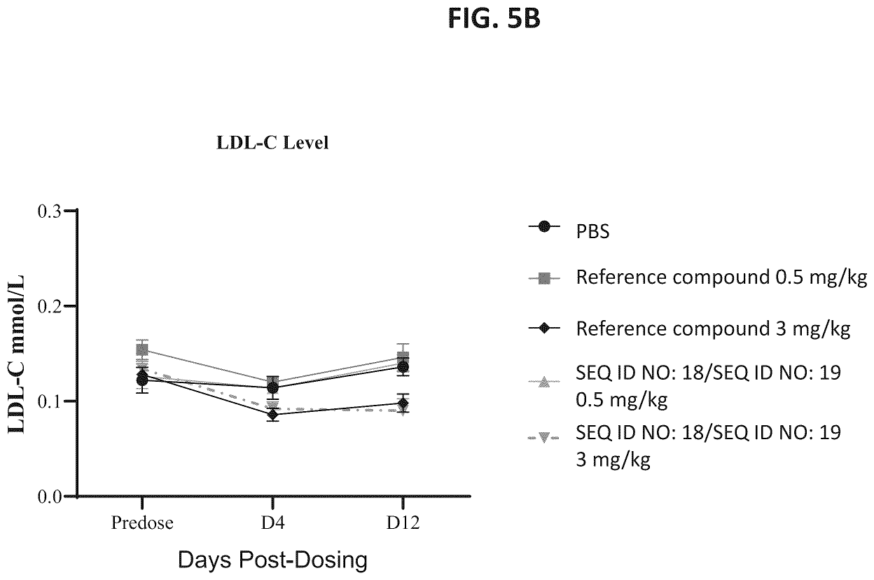

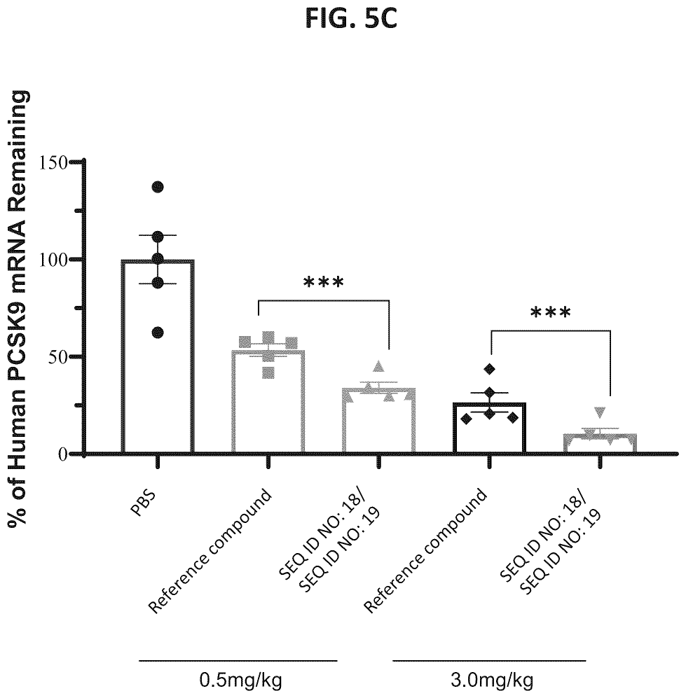

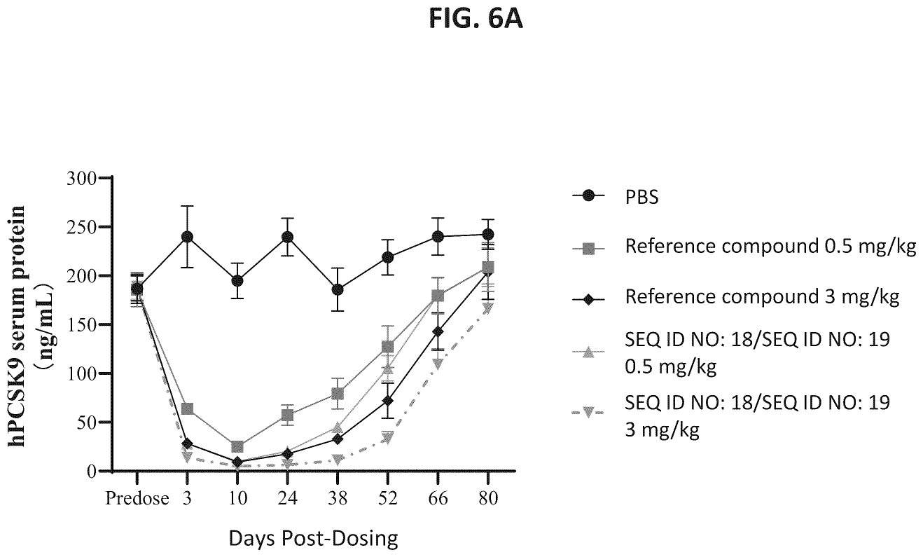

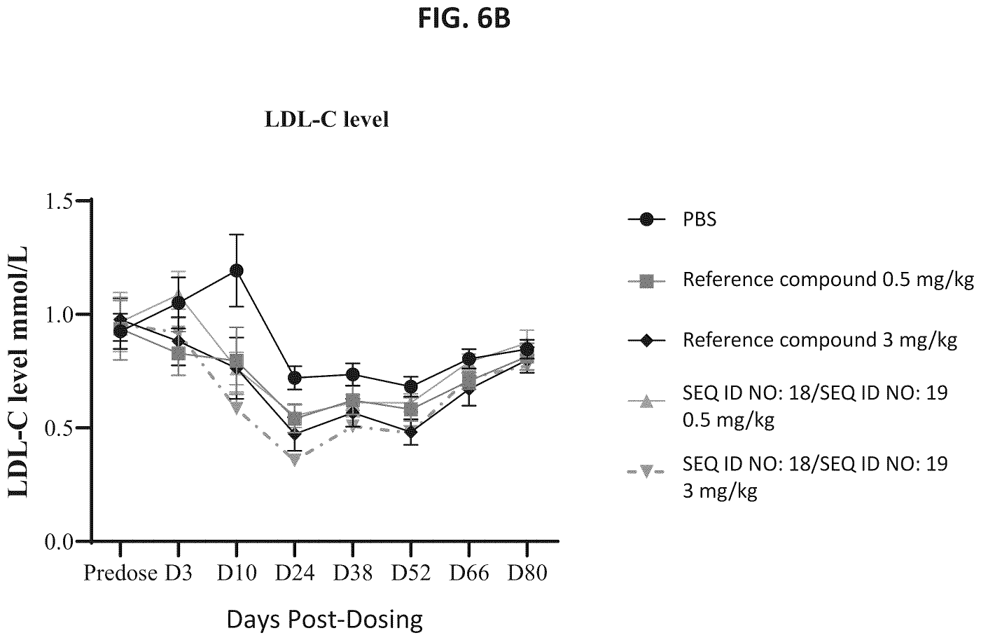

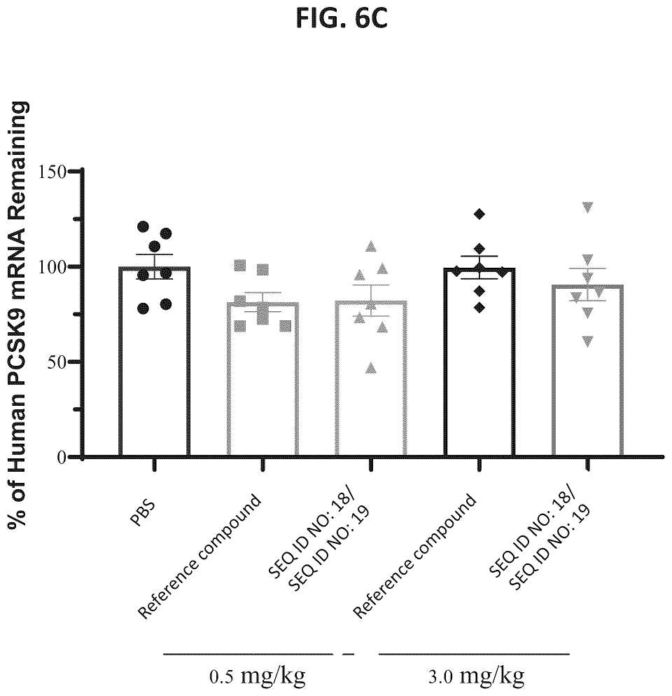

Humanized PCSK9 transgenic mice were administered compounds listed in Table 3 to test the potency and the efficacy of the compounds. The humanized PCSK9 transgenic mice were mice (hsPCSK9 Tg line, GemPharmatech Company, B6/JGpt-Pcsk9em1Cin(hPCSK9-UTR)/Gpt, T053388), in which the endogenous mouse PCSK9 locus was replaced with human PCSK9. These mice were dosed with a reference compound or candidate compounds at 0.5 or 3 mg/kg subcutaneously. In the potency evaluation study (FIGS. 5A-5C) hsPCSK9 Tg mice were fed with chow diet throughout the study. In the efficacy study (FIGS. 6A-6C), hsPCSK9 Tg mice were fed on 60% high-fat diet (Research diets Diet, D12492) over 11 weeks. On the high-fat diet, hypercholesterolemia was successfully established with LDL-C elevated by 3.5-fold on average and remained stable from week 6 to week 11. Animals were maintained on the same diet until the end of the study (Day 80 post-dose).

The reference compound was an oligonucleotide comprising a sense strand and an antisense strand as put forth below:

Sense Strand:
(SEQ ID NO: 56)
5′-Cms-Ums-Am-Gm-Am-Cm-Cf-Um-Gf-Um-dT-Um-Um-Gm-Cm-
Um-Um-Um-Um-Gm-Um-L96-3′
Antisense Strand:
(SEQ ID NO: 57)
3′-Ams-Ams-Gm-Am-Um-Cf-Um-Gf-Gm-Af-Cm-Af-Am-Af-Am-
Cf-Gm-Af-Af-Af-Ams-Cfs-Am-5′,

wherein “Af” is adenine 2′-F ribonucleotide; “Cf” is cytosine 2′-F ribonucleotide; “Gf” is guanine 2′-F ribonucleotide; “Am” is adenine 2′-OMe ribonucleotide; “Cm” is cytosine 2′-OMe ribonucleotide; “Gm” is guanine 2′-OMe ribonucleotide; “Um” is uracil 2′-OMe ribonucleotide; “dT” is thymidine; “s” is phosphorothioate; and “L96” is triantennary GalNAc (N-acetyl-galactosamine) having the following structure:

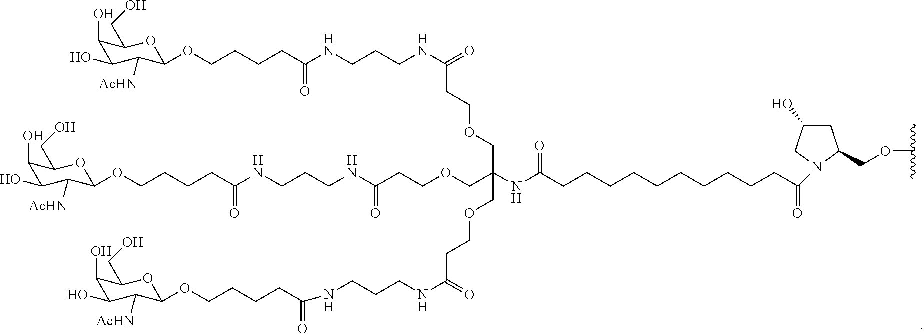 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

### Serum PCSK9 Protein and LDL-Cholesterol Measurement

Cynomolgus serum PCSK9 protein was measured using Human PCSK9 ELISA Kit or Human Pro protein Convertase 9/PCSK9 Immunoassay. LDL-c levels were measured with chemistry analyzer.

For the experiments with the humanized PCSK9 transgenic mice, orbital blood was collected into EDTA-coated tubes after 4-hour fasting at pre-dose and post-dose day 4, 8 and 12. Plasma was separated and collected after centrifugation at 5,000 rpm for 10 minutes. Plasma human PCSK9 protein was measured using a human PCSK9 ELISA Kit.

### Results and Observations

The results showed that the compounds were able to reduce the level of human PCSK9 mRNA in transfected Huh-7 cells by more than 50% at a concentration of 0.02 nM, 0.1 nM, 0.5 nM or 1.5 nM, as shown in Table 2. Also, several compounds were able to reduce the level of human PCSK9 mRNA in transfected cells by greater than 80% or more than 85% at a concentration of 0.5 nM (Table 2). FIGS. 1A and 1B show the results of in vivo compound screening in HDI-mouse liver. All compounds showed efficacy in reducing the RNA expression level of human PCSK9.

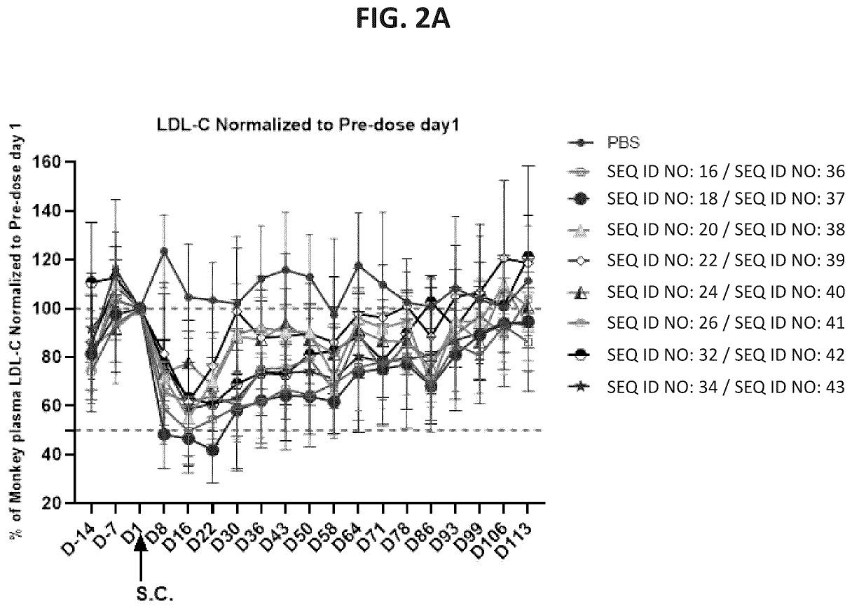

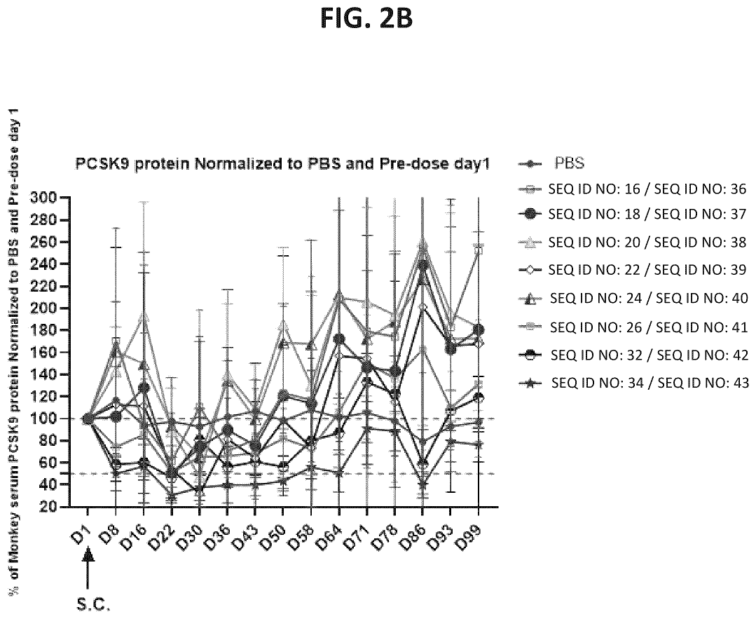

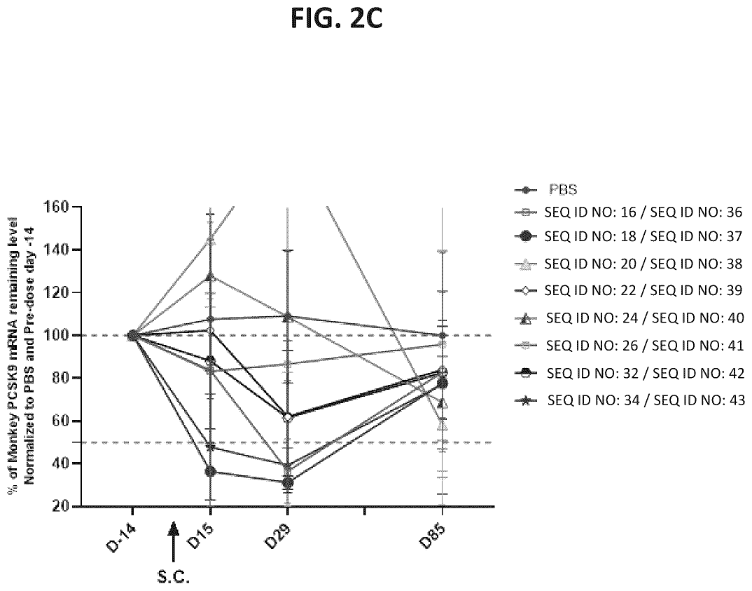

As shown in FIGS. 2A-2C, all eight compounds without the 5′ MeEP cap listed in Table 3 (SEQ ID NO: 16/SEQ ID: NO: 36; SEQ ID NO: 18/SEQ ID NO: 37; SEQ ID NO: 20/SEQ ID: NO: 38; SEQ ID NO: 22/SEQ ID NO: 39; SEQ ID NO: 24/SEQ ID: NO: 40; SEQ ID NO: 26/SEQ ID NO: 41; SEQ ID NO: 32/SEQ ID: NO: 42; and SEQ ID NO: 34/SEQ ID NO: 43) were able to reduce cynomolgus PCSK9 protein in Macaca fascicularis liver samples after a 3 mg/kg single subcutaneous dosing. The LDL-c levels (normalized to pre-dose day 1) remained reduced out to 113 days post-dosing (Day 113, D113), as shown in FIG. 2A. The PCSK9 protein levels (normalized to pre-dose day 1) remained reduced out to 99 days post-dosing (Day 99, D99), as shown in FIG. 2B. As shown in FIG. 2C, by Day 15, several compounds had reduced PCSK9 protein levels by at least 50% relative to pre-dosing levels (Day −14). The maximum reduction in PCSK9 protein levels for all compounds was seen at Day 29 post-dosing, as shown in FIG. 2C.

As shown in FIGS. 3A-3C, three compounds as shown in Table 3 (SEQ ID NO: 16/SEQ ID: NO: 17; SEQ ID NO: 18/SEQ ID NO: 19; and SEQ ID NO: 45/SEQ ID: NO: 19) were able to reduce cynomolgus PCSK9 protein in Macaca fascicularis liver samples after a 3 mg/kg single subcutaneous dosing. The LDL-c levels (normalized to pre-dose day 1) remained reduced out to 112 days post-dosing (Day 112, D112), as shown in FIG. 3A. The PCSK9 protein levels (normalized to pre-dose day 1) remained reduced out to 112 days post-dosing (Day 112, D112), as shown in FIG. 3B. As shown in FIG. 3C, by Day 15, all three tested compounds had reduced PCSK9 protein levels by at least 75% relative to pre-dosing levels (Day −14). This level of PCSK9 protein level reduction was seen at Day 112 post-dosing, as shown in FIG. 3C, demonstrating persistent reduction of protein levels, which may be useful for therapeutic applications in human studies.

As shown in FIGS. 4A and 4B, two compounds as shown in Table 3 (SEQ ID NO: 18/SEQ ID NO: 37; and SEQ ID NO: 18/SEQ ID NO: 19) were tested ex vivo potency in primary PHH as described above. The compounds were able to reduce the level of PCSK9 mRNA in primary human hepatocytes (PHH) ex vivo at about 50% at 10 nM. A dose dependent reduction of PCSK9 mRNA levels was shown in PHH using SEQ ID NO: 18/SEQ ID NO: 19 at eight different doses. At a 500 nM dose, the PCSK9 mRNA level was reduced by over 75%. The dose dependency is advantageous for allowing the ability to fine tune the dosing and levels of PCSK9 mRNA expression in therapeutic applications in humans.

As shown in FIGS. 5A-5C, the compounds comprising SEQ ID NO: 18/SEQ ID NO: 19 (as put forth in Table 3) were able to reduce the levels of human PCSK9 protein and mRNA when administered to humanized PCSK9 transgenic mice who were fed a chow diet as described above. As shown in FIG. 5A, the compound of Table 3 reduced the amount of human PCSK9 (hPSCK9) protein in an amount equal to or greater than the reference compound at the same dosage. As shown in FIG. 5B, LDL-C levels in the mice remained relatively stable throughout the course of the study. Finally, as shown in FIGS. 5C, the compound of Table 3 reduced the amount of hPCSK9 mRNA to a greater degree than the reference compound.

As shown in FIGS. 6A-6C, the compounds comprising SEQ ID NO: 18/SEQ ID NO: 19 (as put forth in Table 3) were able to reduce the levels of hPCSK9 protein, hPCSK9 mRNA, and LDL-C levels when administered to humanized PCSK9 transgenic mice who were fed a 60% high-fat diet which induced hypercholesterolemia, as described above. As shown in FIG. 5A, the compound of Table 3 reduced the amount of hPCSK9 protein in an amount equal to or greater than the reference compound at the same dosage. As shown in FIG. 5B, hypocholesterolemia was induced in these mice and the administration of the compound of Table 3 resulted in a decrease in LDL-C levels over the course of the study. Finally, as shown in FIG. 5C, the compound of Table 3 reduced the amount of hPCSK9 mRNA over the course of the study.

As shown in FIGS. 7A-7B the compounds comprising SEQ ID NO: 18/SEQ ID NO: 19 (as put forth in Table 3) were able to reduce the levels of PCSK9 protein and LDL-C levels in Macaca fascicularis with LDL-c levels greater than 1.5 mmol/L, which were used as a model for spontaneous hypercholesterolemia, as described above.

These results demonstrate several siRNA compounds disclosed herein are effective at reducing human PCSK9 mRNA in vitro in multiple cells including human primary hepatocytes ex vivo. The compounds were also able to reduce cynomolgus PCSK9 mRNA and protein in vivo following a single subcutaneous dose.

TABLE 2
Exemplary Sequences of the Present Application and Their in vitro Potency in Silencing Human
PCSK9 mRNA

| | | | | | | | | % human | | % human | | % human | | % human | |
|---|---|---|---|---|---|---|---|---|---|---|---|---|---|---|---|
| Guide | | | Guide | | Passenger | | | mRNA | | mRNA | | mRNA | | mRNA | |
| Strand | | | strand | SEQ | strand | | SEQ | remaining | | remaining | | remaining | | remaining | |
| posi- | Guide | Guide | sequence | ID | sequence | | ID | at 0.02 nM | | at 0.1 nM | | at 0.5 nM | | at 1.5 nM | |
| tion | start | end | (5′-3′) | NO | (5′-3′) | | NO | Mean | SEM | Mean | SEM | Mean | SEM | Mean | SEM |
| 3564 | 3543 | 3563 | UUAUCUUCAAG | 2 | CUUUUGUAACU | 8 | 3564 | 31.17 | 1.90 | 29.63 | 3.09 | 8.33 | 0.92 | 15.23 | 3.52 |
| | | | UUACAAAAGCA | | UGAAGAUAA | | | | | | | | | | |
| 3571 | 3550 | 3570 | UGAAUAAAUAU | 3 | AACUUGAAGAU | 9 | 3571 | 39.43 | 2.39 | 23.90 | 2.31 | 20.63 | 1.62 | 22.70 | 1.57 |
| | | | CUUCAAGUUAC | | AUUUAUUCA | | | | | | | | | | |
| 3590 | 3569 | 3589 | UAAAAAUGCUA | 4 | UGGGUUUUGUA | 10 | 3590 | 41.47 | 1.67 | 24.87 | 1.47 | 16.50 | 1.17 | 19.90 | 1.27 |
| | | | CAAAACCCAGA | | GCAUUUUUA | | | | | | | | | | |
| 3593 | 3572 | 3592 | UAAUAAAAAUG | 5 | GUUUUGUAGCA | 11 | 3593 | 47.10 | 9.75 | 21.47 | 1.27 | 16.90 | 1.48 | 17.90 | 1.87 |
| | | | CUACAAAACCC | | UUUUUAUUA | | | | | | | | | | |
| 3595 | 3574 | 3594 | UUUAAUAAAAA | 6 | UUUGUAGCAUU | 12 | 3595 | 32.00 | 1.29 | 19.03 | 1.11 | 14.27 | 1.71 | 17.40 | 1.55 |
| | | | UGCUACAAAAC | | UUUAUUAAA | | | | | | | | | | |
| 3597 | 3576 | 3596 | UUAUUAAUAAA | 7 | UGUAGCAUUUU | 13 | 3597 | 27.57 | 2.11 | 21.47 | 1.94 | 9.90 | 1.35 | 12.57 | 2.50 |
| | | | AAUGCUACAAA | | UAUUAAUAA | | | | | | | | | | |
| 3586 | 3565 | 3585 | UAUGCUACAAA | 28 | AUUCUGGGUUU | 30 | 3586 | 41.27 | 3.48 | 28.73 | 0.96 | 14.13 | 1.32 | 18.10 | 1.91 |
| | | | ACCCAGAAUAA | | UGUAGCAUA | | | | | | | | | | |
| 3594 | 3573 | 3593 | UUAAUAAAAAU | 29 | UUUUGUAGCAU | 31 | 3594 | 26.73 | 2.08 | 18.17 | 1.63 | 12.70 | 0.96 | 15.43 | 2.50 |
| | | | GCUACAAAACC | | UUUUAUUAA | | | | | | | | | | |

TABLE 3
Exemplary Sequences of the Present Application

| | | |
|---|---|---|
| G (5′-3′) | [mUs][fUs][fA][mU][fC][mU][fU][mC][mA][fA][mG][mU][mU][fA][mC][fA][mA][mA][mA][mGs][mCs][mA] | SEQ ID NO: 36 |
| P (5′-3′) | [mCs][mUs][mU][mU][mU][fG][mU][fA][fA][fC][fU][mU][mG][mA][mA][mG][mA][mUs][mAs][mA][G1b][G1b][G1b] | SEQ ID NO: 16 |
| G (5′-3′) | [mUs][fGs][fA][mA][fU][mA][fA][mA][mU][fA][mU][mC][mU][fU][mC][fA][mA][mG][mU][mUs][mAs][mC] | SEQ ID NO: 37 |
| P (5′-3′) | [mAs][mAs][mC][mU][mU][fG][mA][fA][fG][fA][fU][mA][mU][mU][mU][mA][mU][mUs][mCs][mA][G1b][G1b][G1b] | SEQ ID NO: 18 |
| G (5′-3′) | [mUs][fAs][fA][mA][fA][mA][fU][mG][mC][fU][mA][mC][mA][fA][mA][fA][mC][mC][mC][mAs][mGs][mA] | SEQ ID NO: 38 |
| P (5′-3′) | [mUs][mGs][mG][mG][mU][fU][mU][fU][fG][fU][fA][mG][mC][mA][mU][mU][mU][mUs][mUs][mA][G1b][G1b][G1b] | SEQ ID NO: 20 |
| G (5′-3′) | [mUs][fAs][fA][mU][fA][mA][fA][mA][mA][fU][mG][mC][mU][fA][mC][fA][mA][mA][mA][mCs][mCs][mC] | SEQ ID NO: 39 |
| P (5′-3′) | [mGs][mUs][mU][mU][mU][fG][mU][fA][fG][fC][fA][mU][mU][mU][mU][mU][mA][mUs][mUs][mA][G1b][G1b][G1b] | SEQ ID NO: 22 |
| G (5′-3′) | [mUs][fUs][fU][mA][fA][mU][fA][mA][mA][fA][mA][mU][mG][fC][mU][fA][mC][mA][mA][mAs][mAs][mC] | SEQ ID NO: 40 |
| P (5′-3′) | [mUs][mUs][mU][mG][mU][fA][mG][fC][fA][fU][fU][mU][mU][mU][mA][mU][mU][mAs][mAs][mA][G1b][G1b][G1b] | SEQ ID NO: 24 |
| G (5′-3′) | [mUs][fUs][fA][mU][fU][mA][fA][mU][mA][fA][mA][mA][mA][fU][mG][fC][mU][mA][mC][mAs][mAs][mA] | SEQ ID NO: 41 |
| P (5′-3′) | [mUs][mGs][mU][mA][mG][fC][mA][f][fU][fU][fU][mU][mA][mU][mU][mA][mA][mUs][mAs][mA][G1b][G1b][G1b] | SEQ ID NO: 26 |
| G (5′-3′) | [mUs][fAs][fU][mG][fC][mU][fA][mC][mA][fA][mA][mA][mC][fC][mC][fA][mG][mA][mA][mUs][mAs][mA] | SEQ ID NO: 42 |
| P (5′-3′) | [mAs][mUs][mU][mC][mU][fG][mG][fG][fU][fU][fU][mU][mG][mU][mA][mG][mC][mAs][mUs][mA][G1b][G1b][G1b] | SEQ ID NO: 32 |
| G (5′-3′) | [mUs][fUs][fA][mA][fU][mA][fA][mA][mA][fA][mU][mG][mC][fU][mA][fC][mA][mA][mA][mAs][mCs][mC] | SEQ ID NO: 43 |
| P (5′-3′) | [mUs][mUs][mU][mU][mG][fU][mA][fG][fC][fA][fU][mU][mU][mU][mU][mA][mU][mUs][mAs][mA][G1b][G1b][G1b] | SEQ ID NO: 34 |
| G (5′-3′) | [mUs][fGs][fA][mA][fU][mA][fA][mA][mU][fA][mU][mC][mU][fU][mC][fA][mA][mG][mU][mUs][mAs][mC] | SEQ ID NO: 37 |
| P (5′-3′) | [mAs][mAs][mC][mU][mU][fG][mA][fA][fG][fA][fU][mA][mU][mU][mU][mA][mU][mU][mC][mA][G1b][G1b][G1b] | SEQ ID NO: 52 |
| G (5′-3′) | [MeEPmUs][fUs][fA][mU][fC][mU][fU][mC][mA][fA][mG][mU][mU][fA][mC][fA][mA][mA][mA][mGs][mCs][mA] | SEQ ID NO: 17 |
| P (5′-3′) | [mCs][mUs][mU][mU][mU][fG][mU][fA][fA][fC][fU][mU][mG][mA][mA][mG][mA][mUs][mAs][mA][G1b][G1b][G1b] | SEQ ID NO: 16 |
| G (5′-3′) | [MeEPmUs][fGs][fA][mA][fU][mA][fA][mA][mU][fA][mU][mC][mU][fU][mC][fA][mA][mG][mU][mUs][mAs][mC] | SEQ ID NO: 19 |
| P (5′-3′) | [mAs][mAs][mC][mU][mU][fG][mA][fA][fG][fA][fU][mA][mU][mU][mU][mA][mU][mUs][mCs][mA][G1b][G1b][G1b] | SEQ ID NO: 18 |
| G (5′-3′) | [MeEPmUs][fAs][fA][mA][fA][mA][fU][mG][mC][fU][mA][mC][mA][fA][mA][fA][mC][mC][mC][mAs][mGs][mA] | SEQ ID NO: 21 |
| P (5′-3′) | [mUs][mGs][mG][mG][mU][fU][mU][fU][fG][fU][fA][mG][mC][mA][mU][mU][mU][mUs][mUs][mA][G1b][G1b][G1b] | SEQ ID NO: 20 |
| G (5′-3′) | [MeEPmUs][fAs][fA][mU][fA][mA][fA][mA][mA][fU][mG][mC][mU][fA][mC][fA][mA][mA][mA][mCs][mCs][mC] | SEQ ID NO: 23 |
| P (5′-3′) | [mGs][mUs][mU][mU][mU][fG][mU][fA][fG][fC][fA][mU][mU][mU][mU][mU][mA][mUs][mUs][mA][G1b][G1b][G1b] | SEQ ID NO: 22 |
| G (5′-3′) | [MeEPmUs][fUs][fU][mA][fA][mU][fA][mA][mA][fA][mA][mU][mG][fC][mU][fA][mC][mA][mA][mAs][mAs][mC] | SEQ ID NO: 25 |
| P (5′-3′) | [mUs][mUs][mU][mG][mU][fA][mG][fC][fA][fU][fU][mU][mU][mU][mA][mU][mU][mAs][mAs][mA][G1b][G1b][G1b] | SEQ ID NO: 24 |
| G (5′-3′) | [MeEPmUs][fUs][fA][mU][fU][mA][fA][mU][mA][fA][mA][mA][mA][fU][mG][fC][mU][mA][mC][mAs][mAs][mA] | SEQ ID NO: 27 |
| P (5′-3′) | [mUs][mGs][mU][mA][mG][fC][mA][fU][fU][fU][fU][mU][mA][mU][mU][mA][mA][mUs][mAs][mA][G1b][G1b][G1b] | SEQ ID NO: 26 |
| G (5′-3′) | [MeEPmUs][fAs][fU][mG][fC][mU][fA][mC][mA][fA][mA][mA][mC][fC][mC][fA][mG][mA][mA][mUs][mAs][mA] | SEQ ID NO: 33 |
| P (5′-3′) | [mAs][mUs][mU][mC][mU][fG][mG][fG][fU][fU][fU][mU][mG][mU][mA][mG][mC][mAs][mUs][mA][G1b][G1b][G1b] | SEQ ID NO: 32 |
| G (5′-3′) | [MeEPmUs][fUs][fA][mA][fU][mA][fA][mA][mA][fA][mU][mG][mC][fU][mA][fC][mA][mA][mA][mAs][mCs][mC] | SEQ ID NO: 35 |
| P (5′-3′) | [mUs][mUs][mU][mU][mG][fU][mA][fG][fC][fA][fU][mU][mU][mU][mU][mA][mU][mUs][mAs][mA][G1b][G1b][G1b] | SEQ ID NO: 34 |
| G (5′-3′) | [MeEPmUs][fGs][fA][mA][fU][mA][fA][mA][mU][fA][mU][mC][mU][fU][mC][fA][mA][mG][mU][mUs][mAs][mC] | SEQ ID NO: 19 |
| P (5′-3′) | [mAs][mAs][mC][mU][mU][fG][mA][fA][fG][fA][fU][mA][mU][mU][mU][mA][mU][mU][mC][mA][G1b][G1b][G1b] | SEQ ID NO: 52 |
| G (5′-3′) | [EPmUs][fGs][fA][mA][fU][mA][fA][mA][mU][fA][mU][mC][mU][fU][mC][fA][mA][mG][mU][mUs][mAs][mC] | SEQ ID NO: 53 |
| P (5′-3′) | [mAs][mAs][mC][mU][mU][fG][mA][fA][fG][fA][fU][mA][mU][mU][mU][mA][mU][mUs][mCs][mA][G1b][G1b][G1b] | SEQ ID NO: 18 |
| G (5′-3′) | [C-EPmUs][fGs][fA][mA][fU][mA][fA][mA][mU][fA][mU][mC][mU][fU][mC][fA][mA][mG][mU][mUs][mAs][mC] | SEQ ID NO: 54 |
| P (5′-3′) | [mAs][mAs][mC][mU][mU][fG][mA][fA][fG][fA][fU][mA][mU][mU][mU][mA][mU][mUs][mCs][mA][G1b][G1b][G1b] | SEQ ID NO: 18 |
| G (5′-3′) | [MeEPmUs][fUs][fA][mU][fC][mU][fU][mC][mA][fA][mG][mU][mU][fA][mC][fA][mA][mA][mA][mGs][mCs][mA] | SEQ ID NO: 17 |
| P (5′-3′) | [mCs][mUs][mU][mU][mU][fG][mU][fA][fA][fC][fU][mU][mG][mA][mA][mG][mA][mUs][mAs][mA][G1bs][G1bs][G1b] | SEQ ID NO: 44 |
| G (5′-33) | [MeEPmUs][fGs][fA][mA][fU][mA][fA][mA][mU][fA][mU][mC][mU][fU][mC][fA][mA][mG][mU][mUs][mAs][mC] | SEQ ID NO: 19 |
| P (5′-3′) | [mAs][mAs][mC][mU][mU][fG][mA][fA][fG][fA][fU][mA][mU][mU][mU][mA][mU][mUs][mCs][mA][G1bs][G1bs][G1b] | SEQ ID NO: 45 |
| G (5′-3′) | [MeEPmUs][fAs][fA][mA][fA][mA][fU][mG][mC][fU][mA][mC][mA][fA][mA][fA][mC][mC][mC][mAs][mGs][mA] | SEQ ID NO: 21 |
| P (5′-3′) | [mUs][mGs][mG][mG][mU][fU][mU][fU][fG][fU][fA][mG][mC][mA][mU][mU][mU][mUs][mUs][mA][G1bs][G1bs][G1b] | SEQ ID NO: 46 |
| G (5′-3′) | [MeEPmUs][fAs][fA][mU][fA][mA][fA][mA][mA][fU][mG][mC][mU][fA][mC][fA][mA][mA][mA][mCs][mCs][mC] | SEQ ID NO: 23 |
| P (5′-3′) | [mGs][mUs][mU][mU][mU][fG][mU][fA][fG][fC][fA][mU][mU][mU][mU][mU][mA][mUs][mUs][mA][G1bs][G1bs][G1b] | SEQ ID NO: 47 |
| G (5′-3′) | [MeEPmUs][fUs][fU][mA][fA][mU][fA][mA][mA][fA][mA][mU][mG][fC][mU][fA][mC][mA][mA][mAs][mAs][mC] | SEQ ID NO: 25 |
| P (5′-3′) | [mUs][mUs][mU][mG][mU][fA][mG][fC][fA ][fU][fU][mU][mU][mU][mA][mU][mU][mAs][mAs][mA][G1bs][G1bs][G1b] | SEQ ID NO: 48 |
| G (5′-3′) | [MeEPmUs][fUs][fA][mU][fU][mA][fA][mU][mA][fA][mA][mA][mA][fU][mG][fC][mU][mA][mC][mAs][mAs][mA] | SEQ ID NO: 27 |
| P (5′-3′) | [mUs][mGs][mU][mA][mG][fC][mA][fU][fU][fU][fU][mU][mA][mU][mU][mA][mA][mUs][mAs][mA][G1bs][G1bs][G1b] | SEQ ID NO: 49 |
| G (5′-3′) | [MeEPmUs][fAs][fU][mG][fC][mU][fA][mC][mA][fA][mA][mA][mC][fC][mC][fA][mG][mA][mA][mUs][mAs][mA] | SEQ ID NO: 33 |
| P (5′-3′) | [mAs][mUs][mU][mC][mU][fG][mG][fG][fU][fU][fU][mU][mG][mU][mA][mG][mC][mAs][mUs][mA][G1bs][G1bs][G1b] | SEQ ID NO: 50 |
| G (5′-3′) | [MeEPmUs][fUs][fA][mA][fU][mA][fA][mA][mA][fA][mU][mG][mC][fU][mA][fC][mA][mA][mA][mAs][mCs][mC] | SEQ ID NO: 35 |
| P (5′-3′) | [mUs][mUs][mU][mU][mG][fU][mA][fG][[C][fA][fU][mU][mU][mU][mU][mA][mU][mUs] [mAs][mA][G1bs][G1bs][G1b] | SEQ ID NO: 51 |
| G (5′-3′) | [MeEPmUs][fGs][fA][mA][fU][mA][fA][mA][mU][fA][mU][mC][mU][fU][mC][fA][mA][mG][mU][mUs][mAs][mC] | SEQ ID NO: 19 |
| P (5′-3′) | [mAs][mAs][mC][mU][mU][fG][mA][fA][fG][fA][fU][mA][mU][mU][mU][mA][mU][mU][mC][mA][G1bs][G1bs][G1b] | SEQ ID NO: 55 |
| G (5′-3′) | [EPmUs][fGs][fA][mA][fU][mA][fA][mA][mU][fA][mU][mC][mU][fU][mC][fA][mA][mG][mU][mUs][mAs][mC] | SEQ ID NO: 53 |
| P (5′-3′) | [mAs][mAs][mC][mU][mU][fG][mA][fA][fG][fA][fU][mA][mU][mU][mU][mA][mU][mUs][mCs][mA][G1bs][G1bs][G1b] | SEQ ID NO: 45 |
| G (5′-3′) | [C-EPmUs][fGs][fA][mA][fU][mA][fA][mA][mU][fA][mU][mC][mU][fU][mC][fA][mA][mG][mU][mUs][mAs][mC] | SEQ ID NO: 54 |
| P (5′-3′) | [mAs][mAs][mC][mU][mU][fG][mA][fA][fG][fA][fU][mA][mU][mU][mU][mA][mU][mUs][mCs][mA][G1bs][G1bs][G1b] | SEQ ID NO: 45 |

G: guide strand, P: passenger strand
“m” indicates 2′-O-methyl modification, “f” indicates a 2′-F modification, “MeEPmU” is a mono methyl protected phosphate mimic linked to a 5′-terminal
uracil (shown below), “EPmU” is a phosphate mimic linked to a 5′-terminal uracil as shown below, “C-EPmU” is a phosphate mimic linked to the 5′-termina
uracil as shown below, “s” is a phosphorothioate internucleotide linkage, and “G1b” is a GalNAc G1b moiety (shown below). 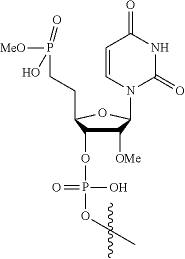
(5′-MeEPmU), 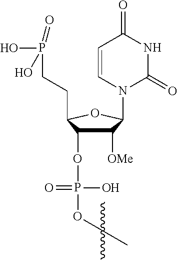
(5′-EPmU), 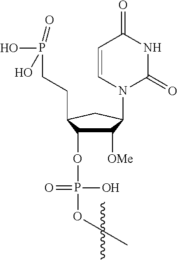
(5′-C-EPmU), 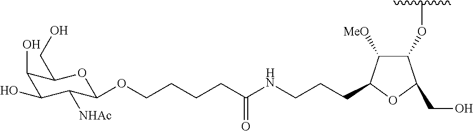 [SMILES: *O[C@H]1[C@@H](OC)[C@H](CCCNC(=O)CCCCOC2OC(CO)C(O)C(O)C2NC(C)=O)O[C@@H]1CO]
(GalNAc G1b)

1. An isolated oligonucleotide comprising a sense strand and an antisense strand, wherein:
the sense strand comprises a nucleotide sequence that is substantially identical to a region between the nucleotide positions 3543 to 3596, from the 5′ end of a Proprotein convertase subtilisin/kexin type 9 (PCSK9) mRNA sequence according to SEQ ID NO: 1,
and the antisense strand is substantially complementary to the sense strand such that the sense strand and the antisense strand together form a double stranded region.

2.-3. (canceled)

4. The isolated oligonucleotide of claim 1, wherein the antisense strand comprises a nucleotide sequence according to any one of: SEQ ID NOs: 2-7, 28 or 29.

5. The isolated oligonucleotide of claim 1, wherein the sense strand comprises a nucleotide sequence according to any one of: SEQ ID NOs: 8-13, 30 or 31.

6. The isolated oligonucleotide of claim 1, wherein the double stranded region comprises:
i) an antisense strand of nucleic acid sequence according to SEQ ID NO: 2 (5′ UUAUCUUCAAGUUACAAAAGCA 3′), and a sense strand of nucleic acid sequence according to SEQ ID NO: 8 (5′ CUUUUGUAACUUGAAGAUAA 3′);
ii) an antisense strand of nucleic acid sequence according to SEQ ID NO: 3 (5′ UGAAUAAAUAUCUUCAAGUUAC 3′), and a sense strand of nucleic acid sequence according to SEQ ID NO: 9 (5′ AACUUGAAGAUAUUUAUUCA 3′);
iii) an antisense strand of nucleic acid sequence according to SEQ ID NO: 6 (5′ UUUAAUAAAAAUGCUACAAAAC 3′), and a sense strand of nucleic acid sequence according to SEQ ID NO: 12 (5′ UUUGUAGCAUUUUUAUUAAA 3′);
iv) an antisense strand of nucleic acid sequence according to SEQ ID NO: 7 (5′ UUAUUAAUAAAAAUGCUACAAA 3′), and a sense strand of nucleic acid sequence according to SEQ ID NO: 13 (5′ UGUAGCAUUUUUAUUAAUAA 3′);
v) an antisense strand of nucleic acid sequence according to SEQ ID NO: 28 (5′ UAUGCUACAAAACCCAGAAUAA 3′), and a sense strand of nucleic acid sequence according to SEQ ID NO: 30 (5′ AUUCUGGGUUUUGUAGCAUA 3′),
i) an antisense strand of nucleic acid sequence according to SEO ID NO: 4 (5′ UAAAAAUGCUACAAAACCCAGA 3′), and a sense strand of nucleic acid sequence according to SEO ID NO: 10 (5′ UGGGUUUUGUAGCAUUUUUA 3′);
ii) an antisense strand of nucleic acid sequence according to SEO ID NO: 5 (5′ UAAUAAAAAUGCUACAAAACCC 3′), and a sense strand of nucleic acid sequence according to SEO ID NO: 11 (5′ GUUUUUGUAGCAUUUUUAUUA 3′); or
iii) an antisense strand of nucleic acid sequence according to SEO ID NO: 29 (5′ UUAAUAAAAAUGCUACAAAACC 3′), and a sense strand of nucleic acid sequence according to SEO ID NO: 31 (5′ UUUUGUAGCAUUUUUAUUAA 3′).

7. (canceled)

8. The isolated oligonucleotide of claim 1, wherein the sense strand or the antisense strand or both comprise one or more modified nucleotide(s).

9. The isolated oligonucleotide of claim 8, wherein the antisense strand comprises a mono methyl protected phosphate mimic (5′-MeEP) or wherein the antisense strand comprises a phosphate mimic linked to a 5′-terminal uracil, wherein the phosphate mimic is selected from:

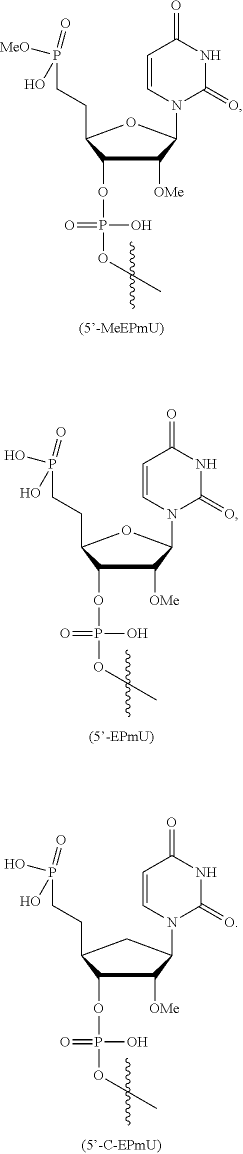

10. (canceled)

11. The isolated oligonucleotide of claim 1, wherein in the sense strand or the antisense strand or both, a terminal or internal nucleotide is linked to a targeting ligand.

12. The isolated oligonucleotide of claim 11, wherein the targeting ligand is attached to the 3′ end (e.g., 3′ terminal position) of the sense strand.

13. The isolated oligonucleotide of claim 11, wherein the targeting ligand comprises a GalNAc.

14. (canceled)

15. The isolated oligonucleotide of claim 1, wherein the antisense strand comprises nucleotides modified with 2′-F modification, and nucleotides modified with 2′-O-methyl modification, according to the formula:
3′(M)₀(F)₀(M)(F)₁(M)₁(F)₁(M)₃(F)₁(M)₂(F)₁(M)₁(F)₁(M)₁(F)₂(M)₁ 5′.

16. The isolated oligonucleotide of claim 1, wherein the sense strand comprises nucleotides modified with 2′-F modification, and nucleotides modified with 2′-O-methyl modification, according to the formula:
5′(M)₀(F)₀(M)₅(F)₁(M)₁(F)₄(M)₉ 3′.

17. The isolated oligonucleotide of claim 15, wherein the antisense strand comprises any one of:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′);
ii) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′);
iii) i) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′);
viii) an antisense strand of nucleic acid sequence according to SEQ ID NO: 35 (5′ [MeEPmUs][fUs][fA][mA][fU][mA][fA][mA][mA][fA][mU][mG][mC][fU][mA][fC][mA][mA][mA][mAs][mCs][mC] 3′);
ix) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′); or
x) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′),
wherein “m” is a 2′-O-methyl modified nucleotide, “f” is a 2′-F modified nucleotide, “s” is a phosphorothioate internucleotide linkage, “MeEPmU” is 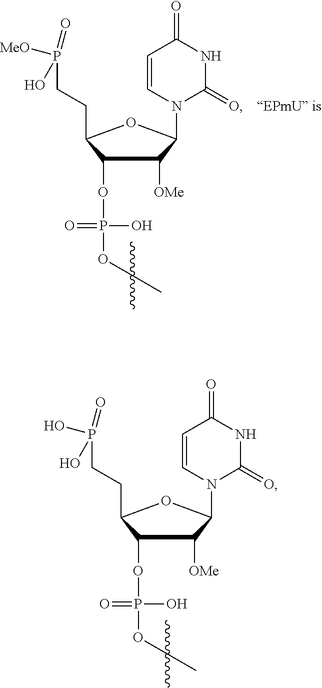
and “C-EPmU” is

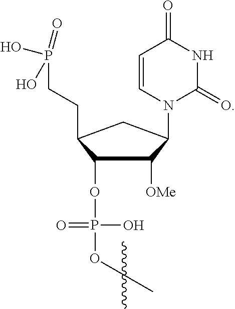

18. The isolated oligonucleotide of claim 16, wherein the sense strand comprises any one of:
i) a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′); or
ix) a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′),
wherein “m” is a 2′-O-methyl modified nucleotide, “f” is a 2′-F modified nucleotide, “s” is a phosphorothioate internucleotide linkage, and “G1b” is a GalNac G1b moiety.

19. The isolated oligonucleotide of claim 17, wherein the double stranded region comprises:
i) an antisense strand of nucleic acid sequence according to SEQ ID NO: 17 (5′ [MeEPmUs][fUs][fA][mU][fC][mU][fU][mC][mA][fA][mG][mU][mU][fA][mC][fA][mA][mA][mA][mGs][mCs][mA] 3′), and
a sense strand of nucleic acid sequence according to SEQ ID NO: 16 (5′ [mCs][mUs][mU][mU][mU][fG][mU][fA][fA][fC][fU][mU][mG][mA][mA][mG][mA][mUs][mAs][mA][G1b][G1b][G1b] 3′);
ii) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′);
iii) i) an antisense strand of nucleic acid sequence according to SEQ ID NO: 21 (5′ [MeEPmUs][fAs][fA][mA][fA][mA][fU][mG][mC][fU][mA][mC][mA][fA][mA][fA][mC][mC][mC][mAs][mGs][mA] 3′), and
a sense strand of nucleic acid sequence according to SEQ ID NO: 20 (5′ [mUs][mGs][mG][mG][mU][fU][mU][fU][fG][fU][fA][mG][mC][mA][mU][mU][mU][mUs][mUs][mA][G1b][G1b][G1b] 3′);
iv) an antisense strand of nucleic acid sequence according to SEQ ID NO: 23 (5′ [MeEPmUs][fAs][fA][mU][fA][mA][fA][mA][mA][fU][mG][mC][mU][fA][mC][fA][mA][mA][mA][mCs][mCs][mC] 3′), and
a sense strand of nucleic acid sequence according to SEQ ID NO: 22 (5′ [mGs][mUs][mU][mU][mU][fG][mU][fA][fG][fC][fA][mU][mU][mU][mU][mU][mA][mUs][mUs][mA][G1b][G1b][G1b] 3′);
v) an antisense strand of nucleic acid sequence according to SEQ ID NO: 25 (5′ [MeEPmUs][fUs][fU][mA][fA][mU][fA][mA][mA][fA][mA][mU][mG][fC][mU][fA][mC][mA][mA][mAs][mAs][mC] 3′), and
a sense strand of nucleic acid sequence according to SEQ ID NO: 24 (5′ [mUs][mUs][mU][mG][mU][fA][mG][fC][fA][fU][fU][mU][mU][mU][mA][mU][mU][mAs][mAs][mA][G1b][G1b][G1b] 3′);
vi) an antisense strand of nucleic acid sequence according to SEQ ID NO: 27 (5′ [MeEPmUs][fUs][fA][mU][fU][mA][fA][mU][mA][fA][mA][mA][mA][fU][mG][fC][mU][mA][mC][mAs][mAs][mA] 3′), and
a sense strand of nucleic acid sequence according to SEQ ID NO: 26 (5′ [mUs][mGs][mU][mA][mG][fC][mA][fU][fU][fU][fU][mU][mA][mU][mU][mA][mA][mUs][mAs][mA][G1b][G1b][G1b] 3′);
vii) an antisense strand of nucleic acid sequence according to SEQ ID NO: 33 (5′ [MeEPmUs][fAs][fU][mG][fC][mU][fA][mC][mA][fA][mA][mA][mC][fC][mC][fA][mG][mA][mA][mUs][mAs][mA] 3′), and
a sense strand of nucleic acid sequence according to SEQ ID NO: 32 (5′ [mAs][mUs][mU][mC][mU][fG][mG][fG][fU][fU][fU][mU][mG][mU][mA][mG][mC][mAs][mUs][mA][G1b][G1b][G1b] 3′);
viii) an antisense strand of nucleic acid sequence according to SEQ ID NO: 35 (5′ [MeEPmUs][fUs][fA][mA][fU][mA][fA][mA][mA][fA][mU][mG][mC][fU][mA][fC][mA][mA][mA][mAs][mCs][mC] 3′), and
a sense strand of nucleic acid sequence according to SEQ ID NO: 34 (5′ [mUs][mUs][mU][mU][mG][fU][mA][fG][fC][fA][fU][mU][mU][mU][mU][mA][mU][mUs][mAs][mA][G1b][G1b][G1b] 3′);
ix) an antisense strand of nucleic acid sequence according to SEQ ID NO: 19 (5′ [MeEPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 52 (5′ [mAs][mAs][mC][mU][mU][fG][mA][fA][fG][fA][fU][mA][mU][mU][mU][mA][mU][mU][mC][mA][G1b][G1b][G1b] 3′);
x) an antisense strand of nucleic acid sequence according to SEQ ID NO: 53 (5′ [EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′); or
xi) an antisense strand of nucleic acid sequence according to SEQ ID NO: 54 (5′ [C-EPmUs][fGs][fA][mA][fU][mA][fA][mA][mU][fA][mU][mC][mU][fU][mC][fA][mA][mG][mU][mUs][mAs][mC] 3′), and
a sense strand of nucleic acid sequence according to SEQ ID NO: 18 (5′ [mAs][mAs][mC][mU][mU][fG][mA][fA][fG][fA][fU][mA][mU][mU][mU][mA][mU][mUs][mCs][mA][G1b][G1b][G1b] 3′),
wherein “m” is a 2′-O-methyl modified nucleotide, “f” is a 2′-F modified nucleotide, “s” is a phosphorothioate internucleotide linkage, “G1b” is a GalNac G1b moiety, “MeEPmU” is 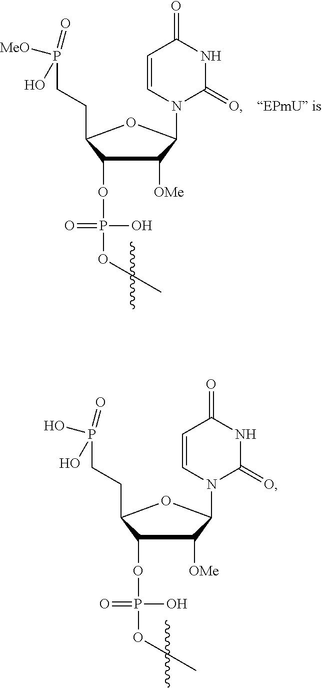
and “C-EPmU” is

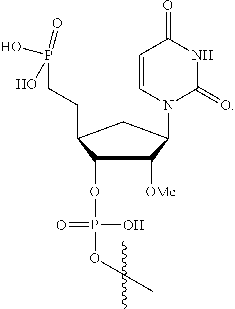

20. A vector encoding the isolated oligonucleotide of claim 1.

21. (canceled)

22. A delivery system comprising the isolated oligonucleotide of claim 1.

23. A pharmaceutical composition comprising the isolated oligonucleotide of claim 1, and a pharmaceutically acceptable carrier, diluent or excipient.

24. (canceled)

25. A method of inhibiting or downregulating the expression or level of PCSK9 or treating or preventing a disease or disorder associated with aberrant or increased expression or activity of PCSK9 or a disease or disorder where PCSK9 plays a role in a subject in need thereof, wherein the method comprises administering to the subject an effective amount of the isolated oligonucleotide of claim 1.

26.-29. (canceled)
